(19) United States
(12) Patent Application Publication
(10) Pub. No.: US 2007/0192134 A1
(43) Pub. Date: Aug. 16, 2007

(54) Clinical decision support system

(76) Inventors: Benjamin Littenberg, Burlington, VT (US); Charles D. MacLean, Shelburne, VT (US); Michael Gagnon, Burlington, VT (US)

Correspondence Address: WOLF GREENFIELD & SACKS, P.C., 600 ATLANTIC AVENUE, BOSTON, MA, 02210-2206, US

(21) Appl. No.: 11/640,103

(22) Filed: Dec. 15, 2006

Related U.S. Application Data
(60) Provisional application No. 60/750,485, filed on Dec. 15, 2005.

(51) Int. Cl. G06Q 10/00; A61B 5/00

(52) U.S. Cl. 705/2; 600/300

(57) ABSTRACT

A clinical decision support system and methods are provided for the management of acute and chronic disorders.

### RELATED APPLICATIONS

This application claims benefit under 35 U.S.C. 119(e) of the filing date of U.S. Ser. No. 60/750,485 filed on Dec. 15, 2005, the entire disclosure of which is incorporated herein by reference.

### FEDERALLY SPONSORED RESEARCH

This invention was made with Government support under National Institutes of Health, Contract/Grant Numbers: R01 DK61167; and K24 DK068380. The Government may have certain rights to this invention.

### FIELD OF THE INVENTION

The present invention relates generally to methods and systems for managing health care.

### BACKGROUND OF THE INVENTION

Diabetes mellitus is one of the most common chronic diseases treated in the United States, affecting almost 8% of the adult population (Mokdad, A. H. et al., 2001, JAMA 2003; 289:76-79; Narayan K. M. et al., JAMA 2003; 290:1884-90). Because diabetes leads to a variety of debilitating complications, it also accounts for a disproportionately high amount of health care spending (Saydah S. H. et al., Am J. Epidemiol 2002; 156:714-19; Gu K. et al., Diabetes Care 1998; 21:1138-45; Economic Costs of Diabetes in the US in 2002, Diabetes Care 2003; 26:917-32). Despite evidence that optimal care can result in reduced complications and improved economic outcomes, such care is often not achieved (Saaddine J. B. et al., Ann Intern Med 2002; 136:565-74; Harris, M. I. et al., Diabetes Care 2000; 23:754-58; Beckles G. L. et al., Diabetes Care 1998; 21:1432-38; Saydah S. H., et al., JAMA 2004; 291:335-42). A recent study of outcomes in diabetic patients from the National Health and Nutrition Examination Survey found that 37% had poor glycemic control (AlC 0.8%), 40% had blood pressure values 0.140/90 mm Hg, and over half had cholesterol levels greater than 200 mg/dL. In total, only 7.3% of patients were on target for all three indicators (Saydah S. H., et al., JAMA 2004; 291:335-42).

Although it is generally accepted that expert, best-practice, clinical guidelines will lead to improvement in clinical care processes and outcomes (Grimshaw J. M., et al., Lancet 1993; 342:1317-22), these effects may not persist without a comprehensive and ongoing system for quality improvement (Goldfarb S., Jt. Comm. J. Qual. Improv. 1999; 25:137-44; Kirkman M. S., et al., Diabetes Care 2002; 25:1946-51; Lomas J. et al., N. Engl. J. Med. 1989:321:1306-11; Renders C. M. et al., Diabetes Care 2001; 24:1821-33). Several studies have reported improvement in outcomes for diabetic patients by using population based, decision support approaches. These studies have been conducted largely in staff-model managed care organizations with robust information systems (Brown J. B. et al., West J. Med. 2000; 172:85-90; McCulloch D. K. et al., Effective Clin. Practice 1998; 1:12-22; Peters A. L., Diabetes Care 1998; 21:1037-43). The majority of health care in the US is, however, delivered in settings where a wide variety of insurance plans are accepted and a central information system is not used.

### SUMMARY OF THE INVENTION

According to one aspect of the invention, a method involving clinical decision support is provided. The method comprises retrieving patient clinical information from a remote data site, performing clinical information interpretation by a guideline-based algorithm, and reporting the clinical information interpretation to a healthcare provider and/or a patient. In one embodiment of the invention, the retrieving of patient clinical information from a remote data site is over a secure network.

In another aspect of the invention, the retrieving of patient clinical information from a remote data site is over a secure network. the clinical decision support comprises automated patient medical report generation, wherein the method is used for managing a medical condition of a patient. The medical condition may be chronic and optionally is a disorder such as diabetes mellitus, cholesterol related disorder, hepatitis, thyroid related disorder or cancer. In one embodiment, the medical condition is diabetes mellitus, and the patient clinical information is a laboratory test data, X-ray data, blood-work data, and/or diagnosis. In yet another embodiment, the patient clinical information is a result from a test such as AlC, serum lipid, urinary microalbumin to creatinine ratio (MCR), and/or serum creatinine.

In yet another embodiment, the remote data site is a laboratory, which includes a point-of-care testing facility. The step of retrieving the patient clinical information may be carried out at a regular time interval, in which the regular time interval is at least once a day, and further in which the guideline-based algorithm is developed from a chronic care model.

In another embodiment, the reporting of clinical information interpretation is carried out by telephone, pager, e-mail, facsimile, mail or via an electronic health record interface. The reporting of clinical information interpretation may be achieved, for instance, using a facsimile report to the healthcare provider, or a mail report for the patient.

According to another aspect of the invention, an automated electronic system for clinical decision support consisting of a storage device for storing patient clinical information; a processor for automatically retrieving the patient clinical information from medical facilities, interpreting the patient clinical information by a guideline-based algorithm; and a processor for sending the clinical information interpretation to a healthcare provider and/or patient. In this system, the clinical decision support is a patient medical report, and the patient clinical information is patient laboratory test data.

According to another aspect of the invention, a computer program product for clinical decision support is provided. The product includes a computer readable code for generating and maintaining a patient registry database; a computer readable code for retrieving clinical information from medical facilities; a computer readable code for interpreting the clinical information and a computer readable code for reporting the interpretation of the clinical information. In an embodiment, the computer program product for clinical decision support is a program for automated medical reporting, and the computer readable code is used for the retrieving of patient clinical information. This may be carried out at regular time intervals. The patient clinical information is laboratory test data, and the interpreting of patient clinical information is guideline-based in some embodiments.

Each of the limitations of the invention can encompass various embodiments of the invention. It is, therefore, anticipated that each of the limitations of the invention involving any one element or combinations of elements can be included in each aspect of the invention.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a diagram that depicts the clinical decision support system (CDSS).

FIG. 2 is a diagram that depicts the steps involved in the initial configuration of laboratories, practices and patients and the data loading sequence in the CDSS.

FIG. 3 is a diagram that depicts the steps involved in the daily operations of the CDSS.

FIG. 4 is a schematic depiction of the operations database.

FIG. 5 is a flow chart depiction of the data site file processing.

FIG. 6 is a flowchart outline of the flow-sheet and alert processing.

FIG. 7 is a flowchart of the reminder processing.

FIG. 8 is a diagram depicting the lab data transfer options.

### DETAILED DESCRIPTION

The invention relates in some aspects to a broad based system to support evidence-based disease management by primary care providers, their practices, and their patients. The system is designed to result in improvements in the process and outcomes of clinical care by, for instance, providing education and feedback to health care providers regarding their patients and to deliver decision support (i.e., flow sheets, alerts and reminders) based on a registry of patients and targeted at primary care providers and patients, to prompt ideal management of disease.

In diseases involving multiple symptoms and therapies, particularly chronic diseases such as diabetes, management of patient care can be quite complex. In practice, patient care in these types of circumstances can fall below threshold targets for optimal care. For instance, in a preliminary study conducted to assess the standard, it was determined that 62.7% of 6,082 diabetic patients had no HbAlc recorded and the mean level in the rest was 8.2% (target value <7.0%). In a sub sample, microalbumin was recorded in only 32% (target 100%). A one-month sample of HbAlc tests ordered by 372 providers on 4,254 patients from 9 participating labs around Vermont produced the following results: the mean HbAlc level was 7.3% (median 7.1, interquartile range 6.0-8.3) and only 49.5% were below target (7.0% or lower). Excluding providers with fewer than 5 patients, the best observed performance was 93% and the worst was 12.5%. Using Achievable Benchmark of Care methodology, the benchmark for fraction below target is 70%. 15% of providers achieved the target.

In order to improve management of health care, the system of the invention was developed. It incorporates 3 basic components: structure, process, and outcome. Structure refers to the resources available to provide health care. These resources include people (nurses, doctors, technicians and other providers), places (hospitals, imaging facilities, clinics, etc.) and things (equipment, supplies, medications, etc.). For instance, in diabetic management, structures include primary, specialty, laboratory and ancillary services (nutritional support, diabetes education, etc.). The system of the invention is a new structural component, a diabetes information system.

Process is the extent to which professionals perform according to accepted standards. It emphasizes what happens to the patient such as prompt delivery of care, appropriate use of tests and treatments, and respectful attention to the patient's needs. The system of the invention improves this aspect of medical care by stimulating both providers and patients to engage in behaviors that are known to improve medical outcomes.

Outcome is the change in the patient's situation following care and includes mortality, functional status, symptoms, satisfaction with care, and costs borne by the patient. Diabetes is particularly interesting because good intermediate outcomes exist that serve as reliable proxies for the long-term outcome patients care. These include control of hyperglycemia, hypertension, hyperlipidemia, and obesity, each of which has convincingly been shown to lead to poorer long-term outcomes.

The clinical decision support system of the invention has three basic components: 1) use of a broad based registry of laboratory-based data to influence patient and provider behavior; 2) reminders to patients with imbedded patient education and decision support; and 3) point-of-decision and office system support for providers evaluating patients in the office.

Although the invention is not limited by any specific advantages, it is believed that the methods of the invention produce several advantages in medical care. The system combines parts of the existing health care system (primary care providers, specialists, clinical laboratories, medical educators, nutritionists, therapists, and patients) in a novel way to make care more coherent. Patients are given tailored information to encourage them to actively manage their own care including self-education, appropriate use of laboratory services, and self-referral to community services with or without the primary provider remembering to initiate the services. Providers are supported to be ready for patient requests and concerns with knowledge, services, and office systems. The system also places recommendations and other decision support material from the guidelines in front of the relevant decision-maker (patient or provider) at the time a decision needs to be made. Healthcare provider training is integrated into the system from the start. Expert consultation is available through expedited access to specialists. In addition, the population-based view of a cohort of patients enables a physician to focus efforts on patients who are typically the most difficult to manage—those who do not receive routine follow-up care.

In some aspects the instant invention provides a clinical decision support system targeted at patients with acute or chronic disorders and the physicians and other healthcare providers who are caring for them in the primary care setting. As used herein, “clinical decision support” refers to the generation of guideline-based recommendations for healthcare providers and/or patients based on the comparison of clinical information to established guidelines for chronic disorders. In a preferred embodiment the healthcare providers are associated with primary care practices. Primary care practices are in general practices where the patient's first point of contact with the healthcare system occurs. Primary care practices are accountable for addressing a large majority of personal health needs, developing a sustained partnership with patients, and practicing in the context of family and community. Because of this, primary care practices are particularly suitable for the methods of the invention. Primary care practices routinely manage a variety of chronic disorders in patients.

The clinical decision support system involves retrieving patient clinical information from a remote data site. As used herein, “clinical information” refers to any source of clinical information regarding the condition of a patient with a chronic disorder. Patient clinical information includes but it is not limited to: laboratory test results including blood, urine, tissues and other excretions and secretions of the body examined for the evidence of chemical imbalance, cellular change, and the presence of pathogenic organisms; medical imaging including X-ray, CAT scan, MRI scan, ultrasound, CT scan; biopsy, laparoscopy, arthroscopy, physical examination, blood pressure, and diagnosis. The clinical information of the invention is indicative of the status of the chronic disorder and is used to evaluate and manage the progression or treatment of the disorder.

For example, the term “laboratory data” refers to laboratory results for medical testing of patients indicative of their condition. The type of laboratory data that is useful in the methods of the invention will depend on the type of disorder being analyzed. The laboratory test data, for example, can measure glycemic control by measuring AlC (measurement of glycosylated hemoglobin); lipid control by measuring total cholesterol trigylceride high density lipoprotein (HDL) or low density lipoprotein (LDL); and renal function by measuring creatinine (a metabolic product that is normally excreted as waste in urine), and microalbumin to creatinine ratio (MCR). In one aspect of the invention, the patient is a diabetic patient, and the clinical information is a laboratory test for AlC, serum lipid tests, urinary microalbumin to creatinine ratio (MCR), and/or serum creatinine. In another embodiment the patient is afflicted with a cholesterol related disorder and the clinical information is test data for LDL, HDL, triglycerides and total cholesterol. In yet another embodiment the patient is a afflicted with a thyroid disorder and the clinical information is physical examination for thyroid gland nodules, test data for blood thyroid hormone levels T4 (thyroxine), T3 (triiodothyronine) and TSH (thyroid stimulating hormone), TPO (thyroperoxidase) antibodies test and ultrasound of the thyroid gland. In another embodiment the patient is afflicted with hepatitis and the clinical information is blood test for hepatitis antigens and/or antibodies, blood tests for alanine aminotransferase (ALT) and aspartate aminotransferase (AST) levels (both are enzymes released when liver cells are injured or die), and liver biopsy. In yet another embodiment the patient is a cancer patient and the clinical information is a laboratory test, imaging or medical procedure directed towards the specific cancer that one of ordinary skill in the art can readily identify. The list of appropriate sources of clinical information for cancer includes but it is not limited to: CT scan, MRI scan, ultrasound scan, bone scan, PET Scan, bone marrow test, barium X-ray, endoscopy, lymphangiogram, IVU (Intravenous urogram) or IVP (IV pyelogram), lumbar puncture, cystoscopy, immunological tests (anti-malignin antibody screen), and cancer marker tests.

The patient clinical information is obtained from a remote data site. A “remote data site” refers to a medical laboratory, diagnostic laboratory, medical facility, medical practice, point-of-care testing device, or any other remote data site capable of generating patient clinical information. The data site is considered remote because it is physically remote from the decision support system/central processing system. In certain embodiments of the invention the remote data site is also physically remote from the location of the healthcare provider and/or practice. In a certain aspect of the invention the remote data site is a point of care site. As used herein, “point-of-care” testing refers to those analytical patient testing activities, provided within a practice but performed outside the physical facilities of the clinical laboratories, i.e. testing that does not require permanently dedicated space. The remote data site stores test information in any format that can be retrieved from a remote location by a file transfer protocol (FTP) in a variety of secure connection methods described herein. In one embodiment, the connections are done by a branch to branch virtual private network (VPN) connections over the internet or private leased data lines. In one embodiment the connections are via wireless internet connections. In one embodiment, the invention also allows for manual data input via secure internet forms software function. The software function accepts the medical record number and test results, and processes them into the registry database. This function allows practices performing point-of-care testing in the office to directly enter test results.

The patient clinical information may be obtained from the remote sites manually or automatically. For simplicity of the system the information is obtained automatically at predetermined or regular time intervals. A regular time interval refers to a time interval at which the collection of the laboratory data is carried out automatically by the methods and systems described herein based on a measurement of time such as hours, days, weeks, months, years etc. In one embodiment of the invention, the collection of data and processing is carried out at least once a day. In one embodiment the transfer and collection of data is carried out once every month, biweekly, or once a week, or once every couple of days. Alternatively the retrieval of information may be carried out at predetermined but not regular time intervals. For instance, a first retrieval step may occur after one week and a second retrieval step may occur after one month. The transfer and collection of data can be customized according to the nature of the disorder that is being managed and the frequency of required testing and medical examinations of the patients.

Preferably the transfer of information occurs over a secure network to maintain patient confidentiality. As used herein “secure network” refers to a network that utilizes secure file transfer and system administration access methods to access files and execute commands on remote servers. It will be appreciated by one of ordinary skill in the art that secure networks can be established in a variety of ways including the utilizations of Telnet, FTP, and SSH. In a certain embodiment of the invention a utility is used wherein commands are encrypted and secure in several ways. For example, both ends of the client/server connection are authenticated using digital certificates, administration access methods are password protected and passwords are protected by being encrypted. Although a secure network is desirable it is not essential since other systems can be arranged for maintaining client confidence, such as through the use of patient codes instead of patient information.

After the patient clinical information is retrieved, clinical information interpretation may be performed using a guideline-based algorithm. As used herein, “clinical information interpretation” refers to the automated comparison of the retrieved laboratory data to a predetermined threshold for designating results. The outcome of this comparison triggers the generation of a certain type of report, as described herein.

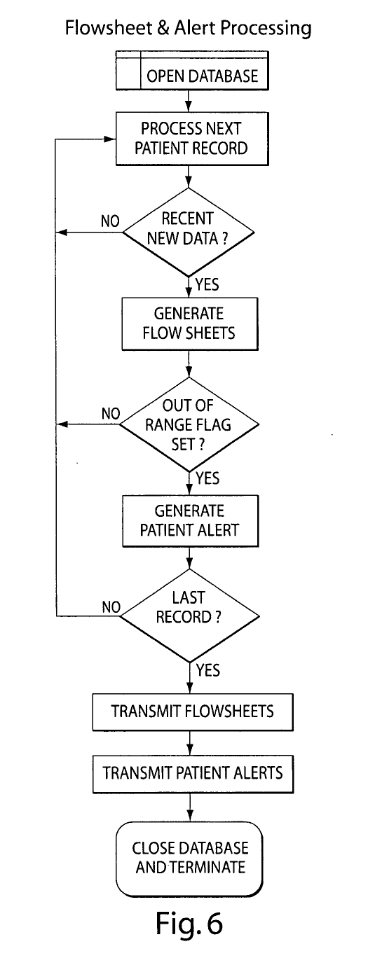

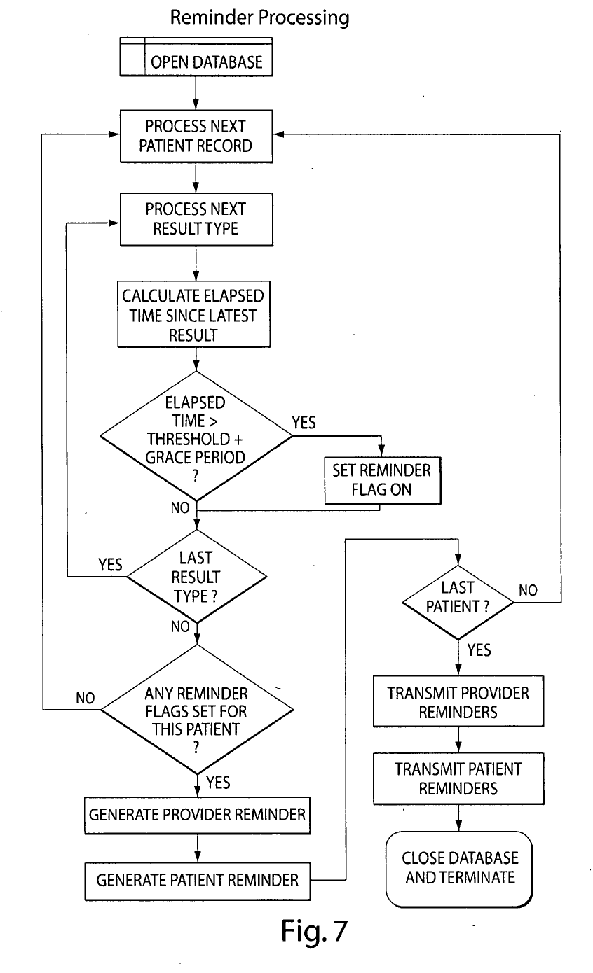

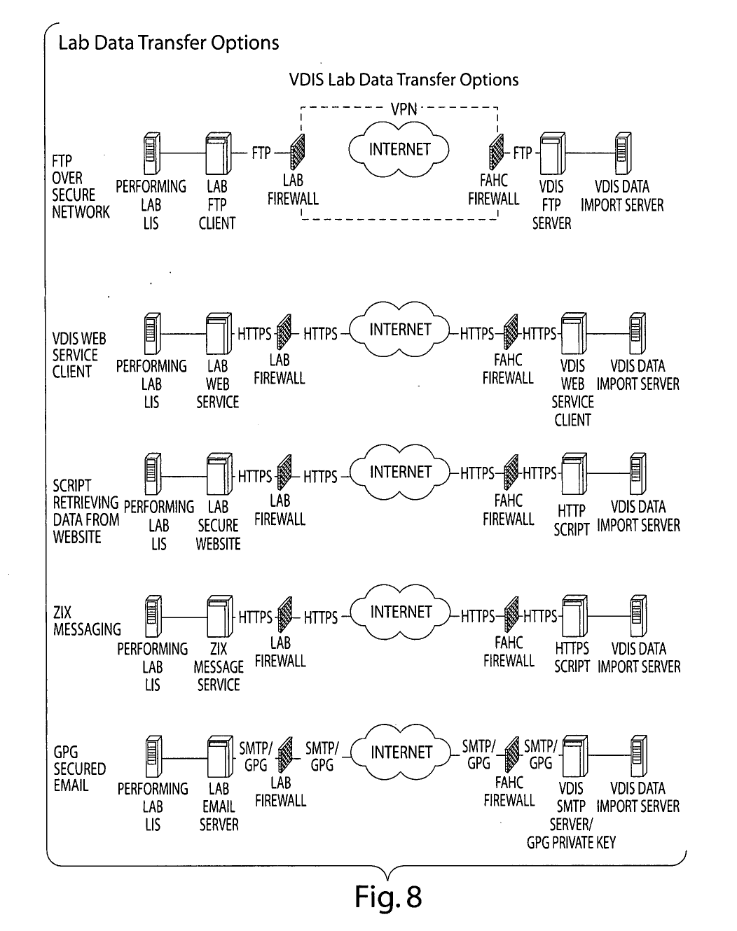

As used herein, the term, “guideline-based algorithm” refers to an algorithm wherein the collected patient clinical information is compared to predetermined threshold values as schematically illustrated by FIG. 6 and FIG. 7. The primary function of the system is to collect pertinent clinical information and to provide accurate and timely flow sheets, reminders and alerts to physicians and their patients. In one embodiment, the patients are diabetes patients. In one embodiment, the system also generates summary population reports for physicians regarding their roster of diabetic patients. In one embodiment, the thresholds for designating a result to be high, are taken from a Vermont guideline, based on the American Diabetes Association Clinical Practice Recommendations for change in therapy: i.e. AlC>8% LDL>130 mg/dL; MCR>300 mg. An AlC is overdue if the previous AlC is more than six months old, or if the previous AlC is 7% or greater and more than three months old. In the example, a one month grace period is allowed, so a patient reminder letter is not generated until seven or four months have elapsed.

Once the information is processed a report of the clinical information interpretation is delivered to a healthcare provider and/or a patient. As used herein, the term “patient” refers to any patient that suffers from an acute or chronic disease or medical condition, the management of which depends upon frequent testing and monitoring of the test results, patient education, etc. In one embodiment, the patient is a diabetic patient. A healthcare provider includes any individual involved in patient management, such as, for instance, nurses, doctors, technicians and other providers that work in hospitals, imaging facilities, clinics, etc.

As used herein “medical report” refers to a report which is generated by the methods and/or systems described herein, and it includes one or more of the following: a flow-sheet faxed to a healthcare provider, a provider alert faxed to the healthcare provider, a patient reminder mailed to the patient, patient alert mailed to the patient, a population report displayed in the browser window and saved under the application root on the production server, and a quarterly population report with reports cards of individual healthcare providers performance mailed to a healthcare provider and/or practice. In one aspect of the invention, the medical report is directed to a healthcare provider. In one embodiment of the invention, the medical report is directed to a primary care practice. In yet another aspect of the invention, the medical report is directed to the patient. In one embodiment, the medical report is a mailed alert when the laboratory test result is above guideline-based threshold. In another embodiment, the medical report is a mailed reminder when the patient is overdue for a recommended laboratory testing. As used herein, the term “reporting” or “report triggering” refers to the generation of a report as described herein, and the communicating of that report via facsimile, e-mail, voicemail, or printed mail to a health care provider or a patient.

The reporting of clinical information interpretation can be also carried out by an “electronic health record interface”. As used herein, electronic health record interface refers to any electronic interface that supports display of electronic database-stored or generated patient information to clinicians and/or patients. As described herein the patient information includes but it is not limited to patient clinical data, test results, clinical notes, prescriptions, scheduling etc.

“Automated patient medical report generation” or “report triggering” refers to the generation of medical reports as described herein by automated means without the requirement for input or active control by a healthcare provider or patient. Automated report generation can be carried out by a central processing unit (CPU), a data processing apparatus or by any other machine capable of collecting data, interpreting data, and generating voice, facsimile, electronic or printed paper reports.

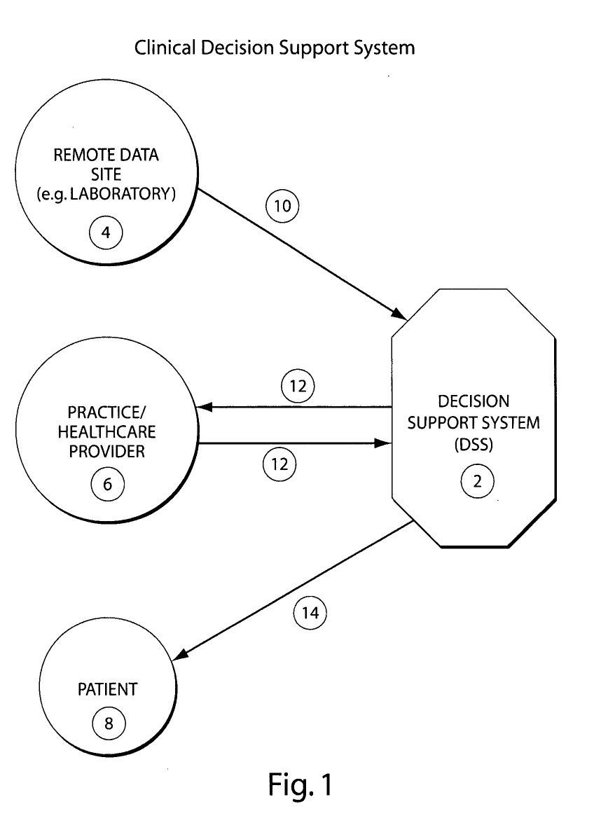

Referring now to FIG. 1, a clinical decision support system for managing the care of patients with chronic disorders according to the instant invention is schematically illustrated. A decision support system/central data processing system 2 is configured to establish communications directly with: a remote data site 4 via communication link 10; a medical practice or healthcare provider 6 via communication link 12; and/or with patient 8 via communication link 14. The remote data site 4 can be a medical laboratory, diagnostic laboratory, medical facility, medical practice, point-of-care testing device, or any other remote data site capable of generating patient clinical information. Patient clinical information includes but it is not limited to laboratory test data, X-ray data, examination and diagnosis. The healthcare provider or practice 6 includes medical services providers, such as doctors, nurses, home health aides, technicians and physician's assistants, and the practice is any medical care facility staffed with healthcare providers. In certain instances the healthcare provider/practice 6 is also a remote data site. Patient 8 is any patient afflicted with a chronic disorder including but not limited to diabetes, cholesterol related disorders, hepatitis, thyroid related disorders and cancer.

The communication links 10, 12, and 14 in the present invention may be established through various methods including FTP over a secure network, web service client, scripts to stimulate HTTP sessions, manual download via an HTTP session, Zix messaging and the use of GPG encryption for secure email. The communication links 10, 12, and 14 in certain instances can also be established via voicemail, email, facsimile and mail. It is understood that the decision support system/central data processing system 2 can be configured to establish communications with a plurality of remote data sites, practices and/or patients. The decision support system/central data processing system 2 is configured to store a registry database of patients with a chronic disorder; retrieve clinical information from the remote data site 4 (or healthcare provider/practice 6) via communication link 10; perform interpretation of the clinical information by an algorithm based on chronic care guideline; and report the clinical information interpretation to the healthcare provider/practice 6 and/or a patient 8 via communication link 12, 14. It will be understood that the decision support system/central data processing system 2 is configured to execute computer program code to perform the methods of the present invention. In certain embodiments the decision support system/central data processing system 2 has one or more processors. Each of these components is described in greater detail herein.

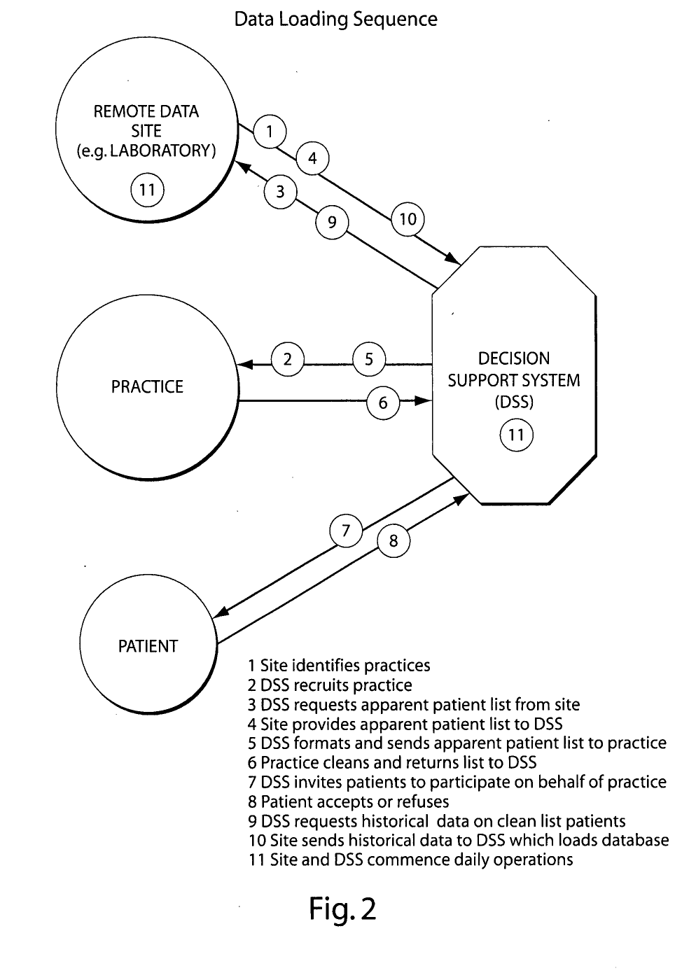

Referring to FIG. 2 a data loading sequence of the present invention is schematically presented. Once the practice is identified by the remote data site 1, the decision support system/central data processing system recruits the practice 2 and requests apparent patient list from the site 3. The remote data site provides the apparent list to the decision support system/central data processing system 4. Next, the decision support system/central data processing system formats and sends the apparent patient list to the practice 5, and the practice reviews and returns the list to the decision support system/central data processing system 6. The decision support system/central data processing system invites the selected patients to participate on behalf of the practice 7, and if the patient accepts the invitation 8, the decision support system/central data processing system requests historical data on the selected “clean” list of patients from the remote site 9. The site sends the historical data on the participating “clean list” patients 10, and the remote data site and the decision support system/central data processing system commence daily operations 11.

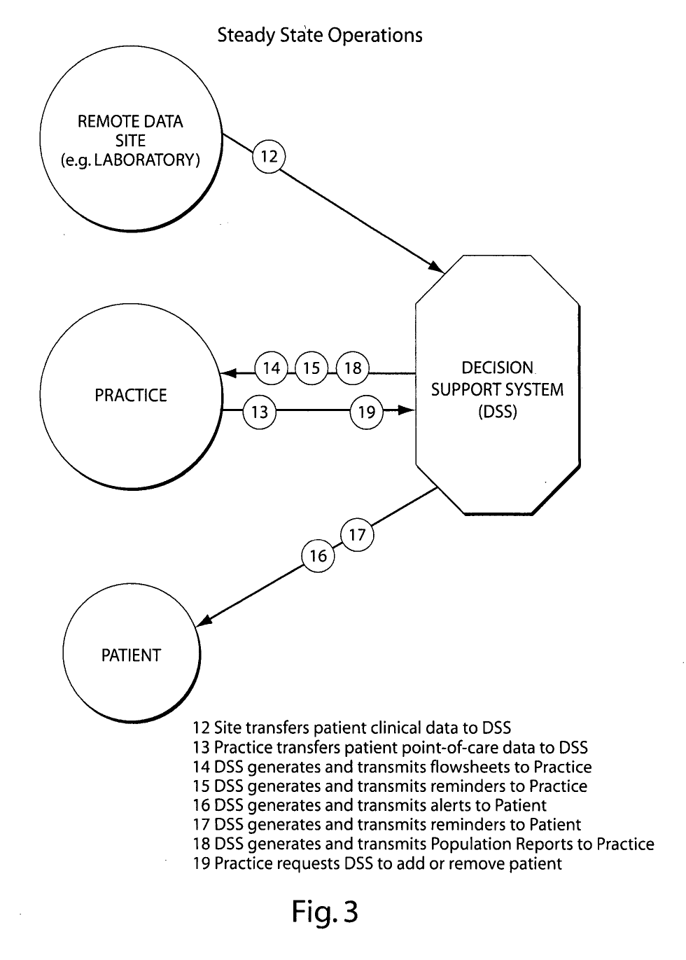

Referring to FIG. 3 the daily “steady state” operations of the methods of the instant invention are schematically depicted. In brief, lab data are uploaded from participating clinical laboratories to the clinical decision support system (CDSS) data registry. Reminders, alerts and population reports are then sent to patients and providers, prompting guideline-based care. In order for patients to be included, they must be cared for in a participating practice. That practice must be using a participating lab, or doing in-office point of care testing in such a way that lab results can be transmitted to CDSS on a timely basis. The remote data site transfers patient clinical data to the decision support system/central data processing system 12, and/or the practice transfers point-of-care data to the decision support system/central data processing system 13.

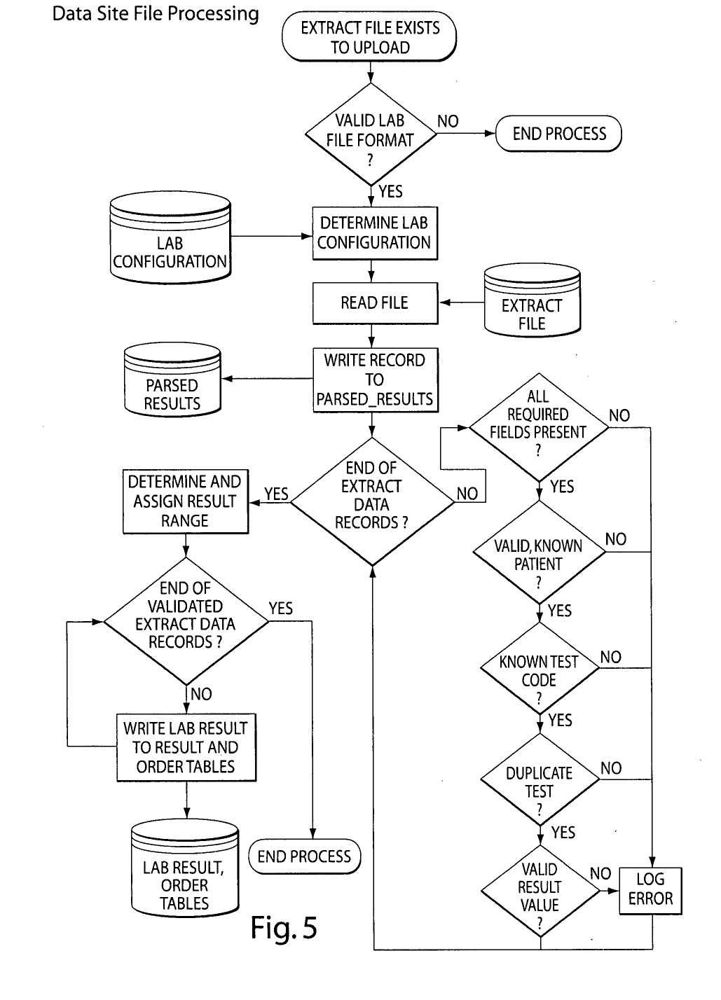

The detailed flowchart for the remote data site file processing is provided in FIG. 5. The decision support system/central data processing system interprets the data by a guideline-based algorithm and generates and transmits flow sheets 14 and/or reminders 15 and/or population reports 18 to practice and/or alerts 16 and/or reminders 17 to patient. Detailed flowcharts for flow-sheet and alert processing and reminder processing are depicted on FIG. 6 and 7, respectively. According to the algorithms of the methods of the invention the patient clinical information is compared to pre-determined values set by established guidelines for chronic care. The outcome of that comparison, herein referred to as interpretation of the clinical information, triggers the generation of a certain decision support report according to the invention as described herein. The practice can request the decision support system/central data processing system to add or remove a patient 19.

For exemplary purposes, the present invention is described in places throughout the disclosure and examples with respect to clinical decision support for patients afflicted with diabetes. However, it is to be understood that the present invention may be utilized with a wide variety of chronic disorders including, but not limited to cholesterol related disorders, hepatitis, thyroid related disorders and cancer.

The details of the database structure and the procedures for the enrollment of labs, practices and patients are described herein. Some of the functions are specific to the research aspects of the CDSS, and others to the general operation of the system. The CDSS involves some of the principles of quality improvement of Donabedian (Donabedian, A., “The Definition of Quality and Approaches to Each Assessment”, Vol I. Ann Arbor Health Administration Press, 1980.) The chronic care model emphasizes the importance of an ideal clinical encounter, a prepared, proactive health care team and an informed, activated patient. Chronic disease registry databases are a central aspect of this model. While other implementations of the chronic care model require substantial investment by the practice and major changes in the providers usual activities, the instant invention is designed to require a minimum of effort, and no financial resources on the part of the providers. The guideline-based algorithm compares the retrieved test data to a guideline-based predetermined value and depending on the outcome of this comparison, it triggers the generation of a certain type of a medical report.

In one embodiment of the invention, a decision support reminder system is provided for primary care practices and their patients with diabetes. In one aspect of the invention, the system has the following components: 1) it uses the chronic care model as an organizing framework; 2) daily data feeds from otherwise independent laboratories; 3) automatic test interpretation using algorithm based on consensus guidelines; 4) use of fax and mail to report to providers and patients not easily reached by electronic networks; and 5) report formats that are accessible and useful to patients and providers.

As used herein “patient registry database” refers to a database of patients characterized by a chronic disorder generated by the methods of invention. Accordingly, the registry database is generated as described herein and schematically illustrated in FIG. 2. It certain embodiments it can be based on patients that have had a particular test or examination that is routinely carried out for a chronic disease. For example, a list of diabetic patients can be developed from patients who have had an AlC test performed in the previous two years. In one embodiment the registry database is built from demographic data entries of selected patients, for example: First Name, Middle Initial, Last Name, Medical Registration Number (MRN), Date of Birth, Gender, Marital Status, Address, Patient Phone Number, Provider (Physician), AlC result and AlC date of service. From the initial list of patients that have undergone a particular test procedure, patients can be further selected based on eligibility criteria such as specific disease, age, care, and cognitive impairment. For example, for diabetic patients initially selected based on AlC tests, the additional criteria include: a) diabetes type I or type II; b) age of 18 or older; c) under the care of a certain PCP for diabetes; d) not suffering from cognitive impairment.

In certain embodiments of the invention it is important that patients do not suffer cognitive impairment because the methods of the instant invention rely on patients to understand reminder and other types of medical reports generated by the methods and systems of the invention, as described herein.

In certain aspects of the invention the registry database comprises one or more of the following components: Operations Database, Practice Database (Access Format) and Web-Data Entry Interface.

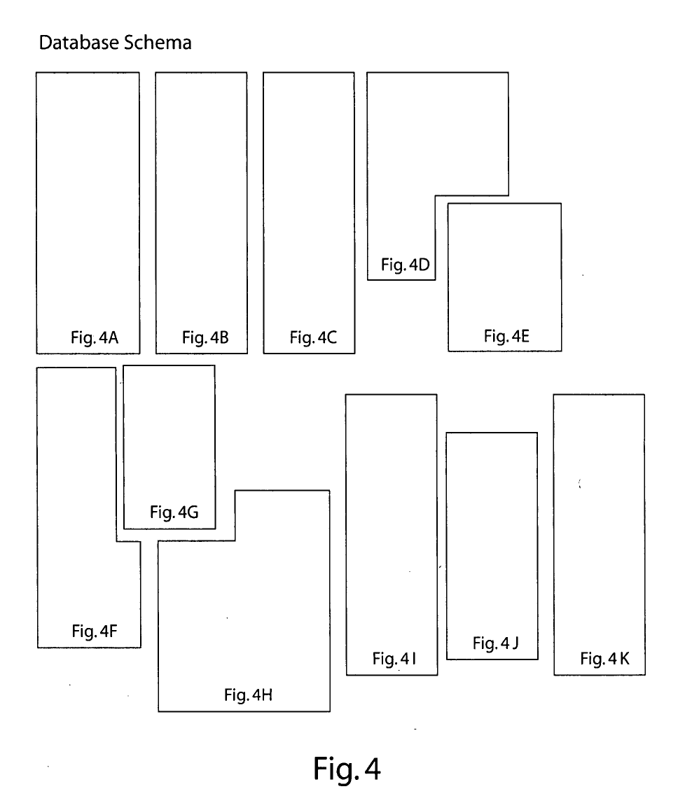

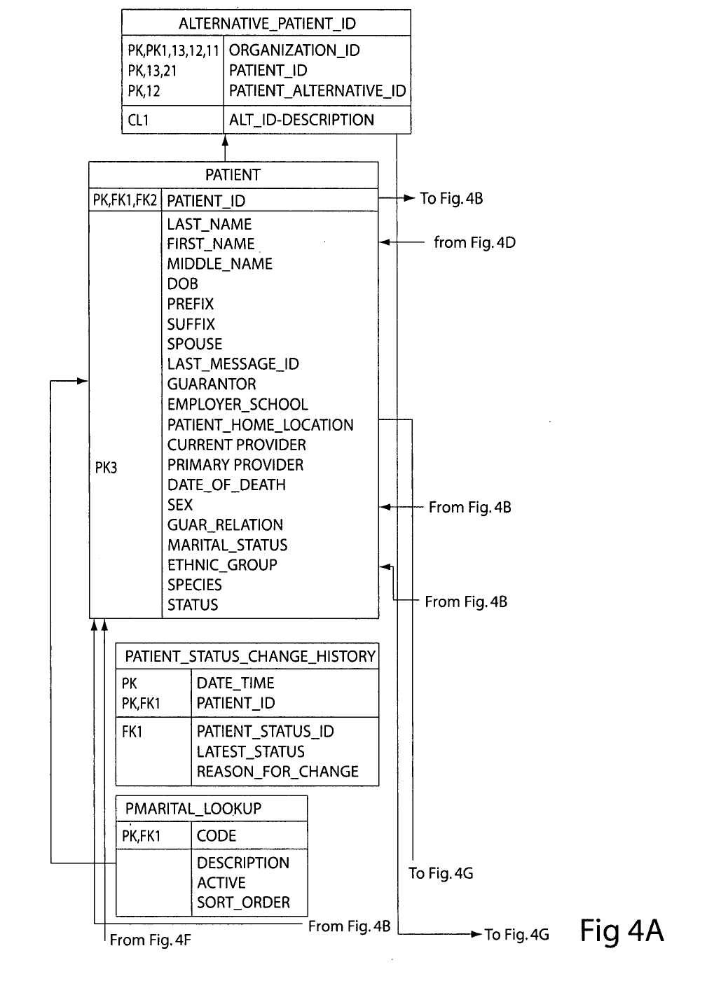

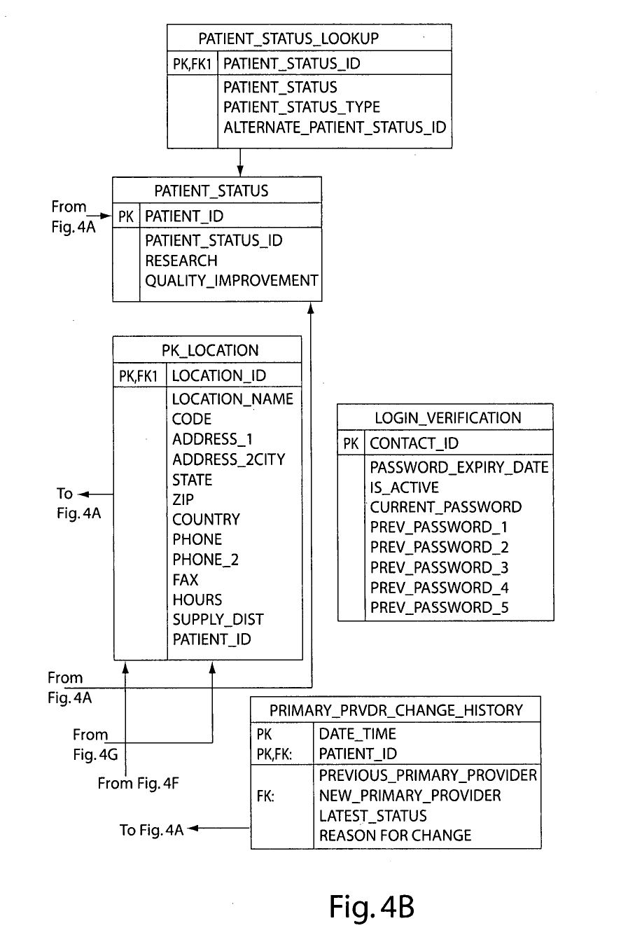

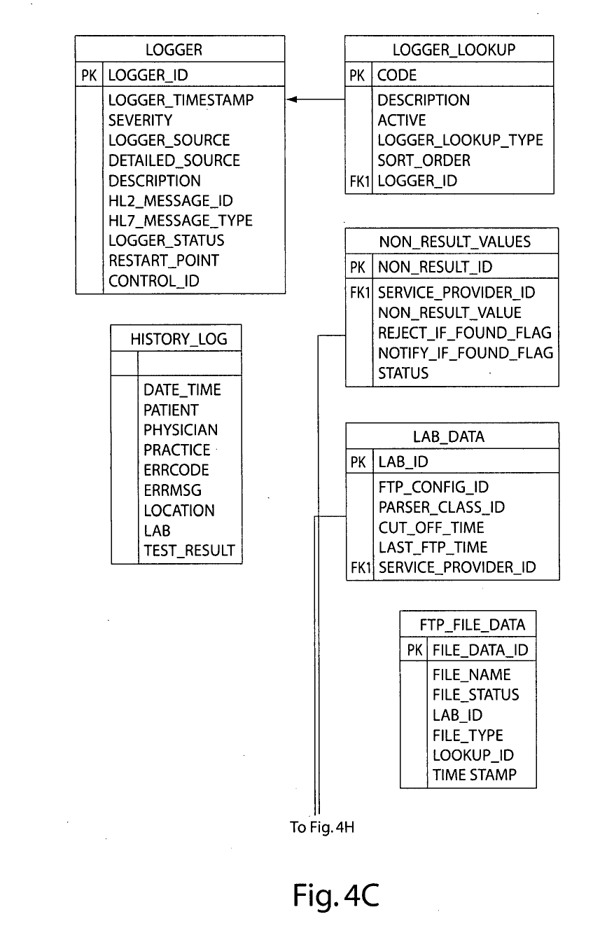

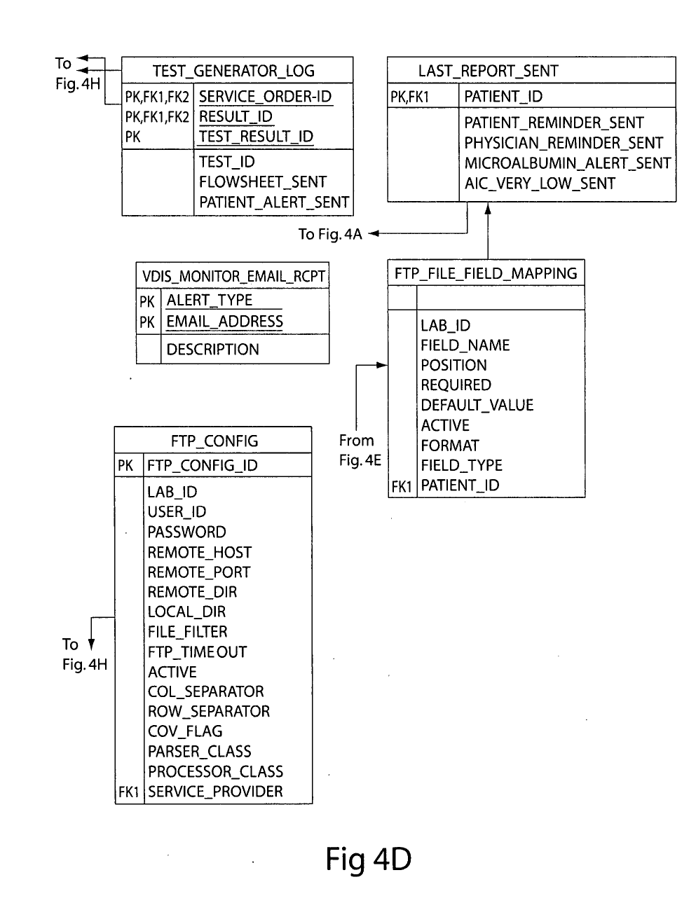

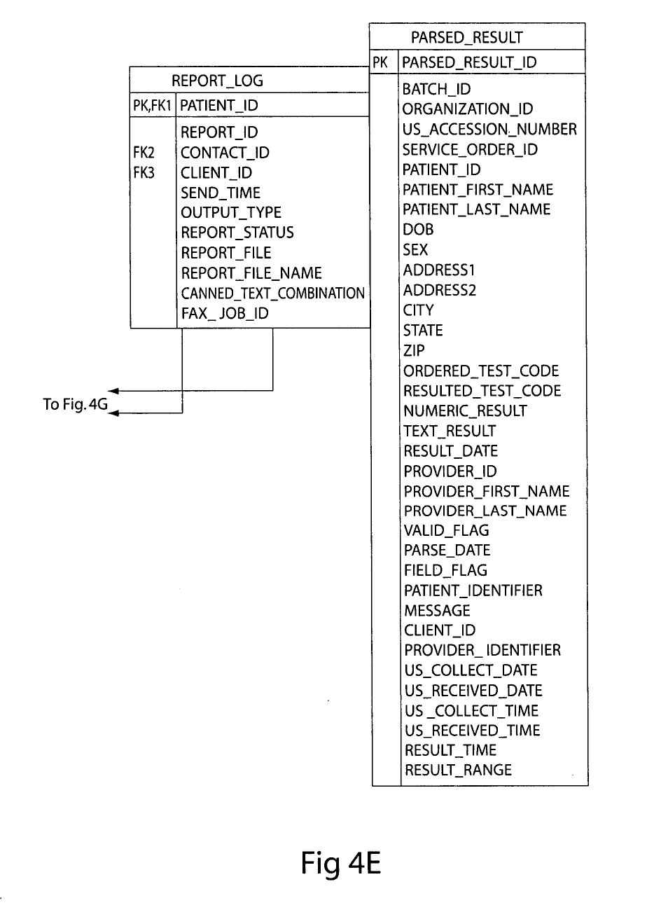

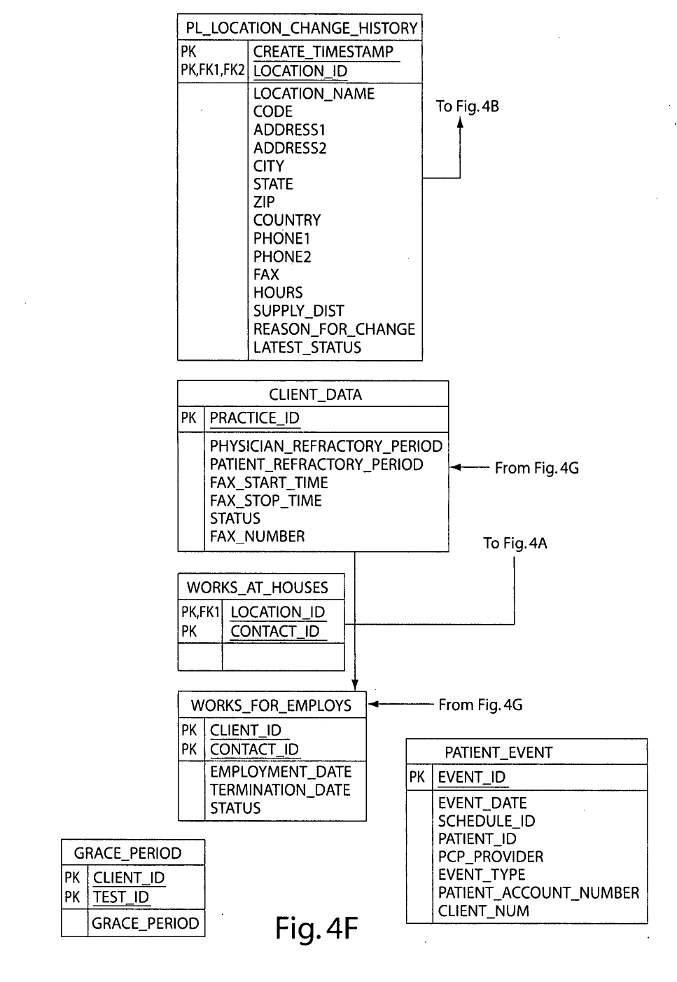

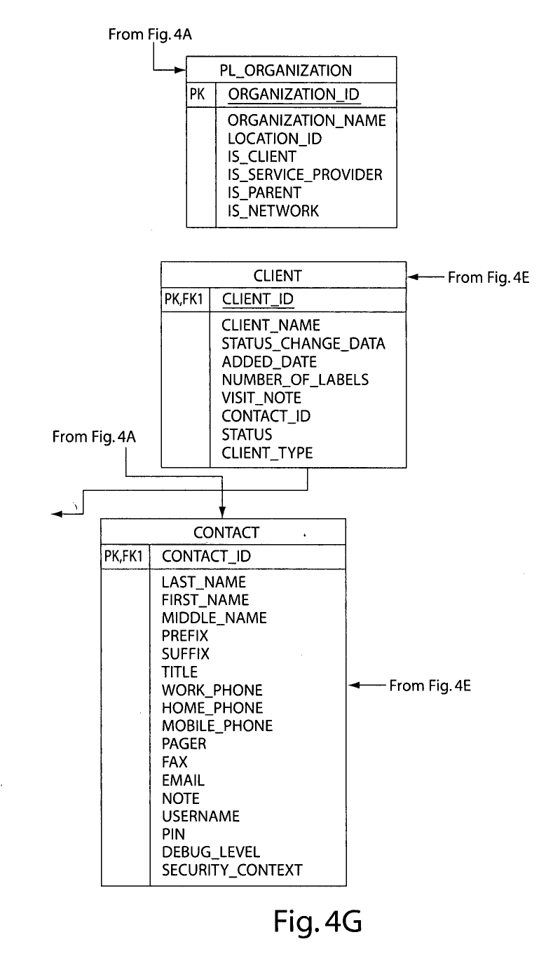

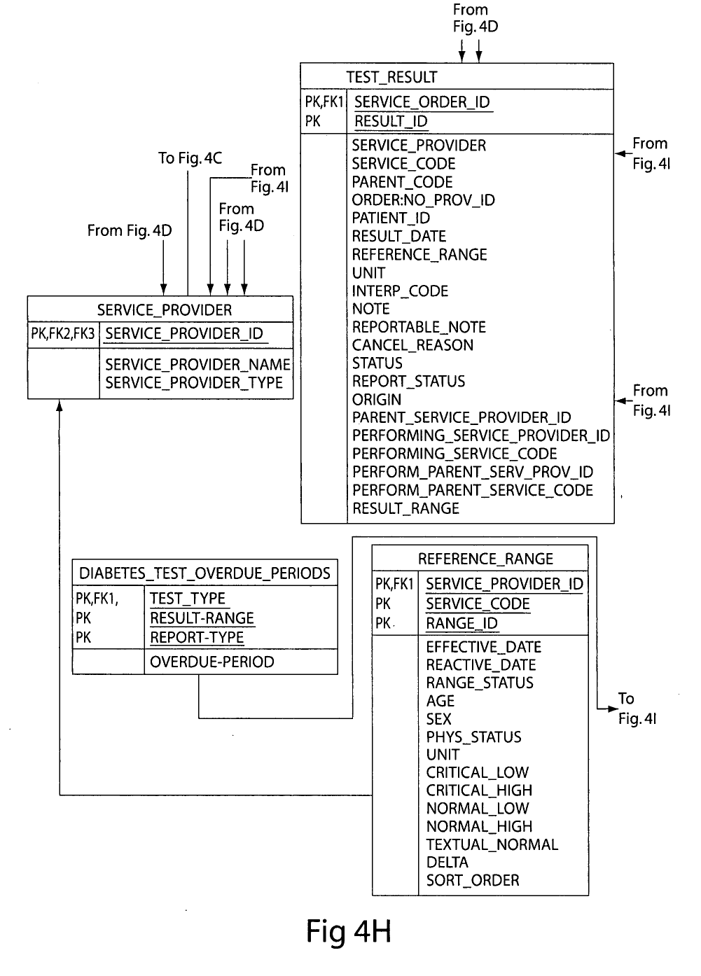

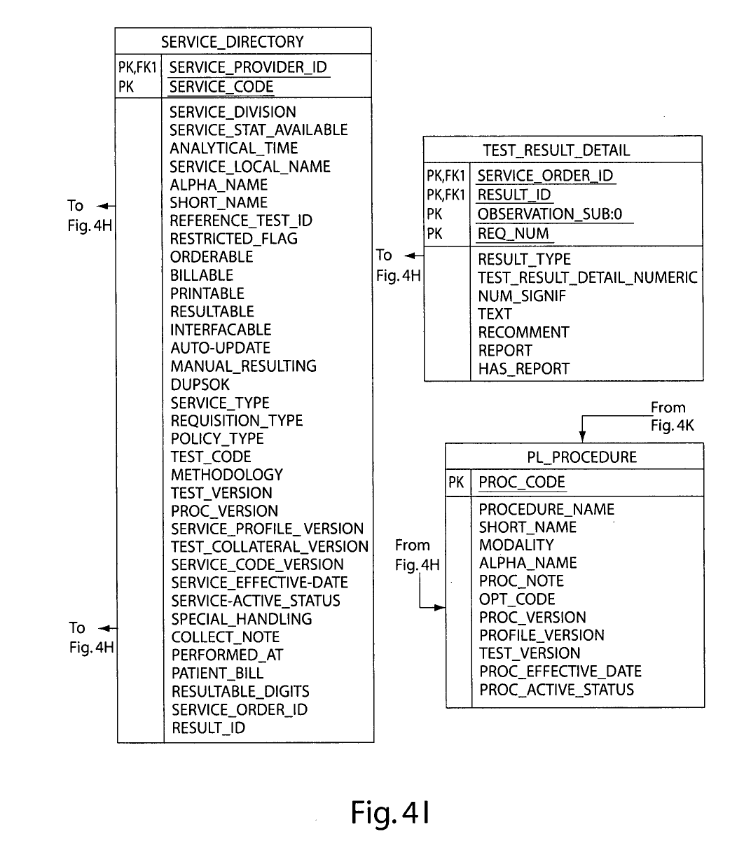

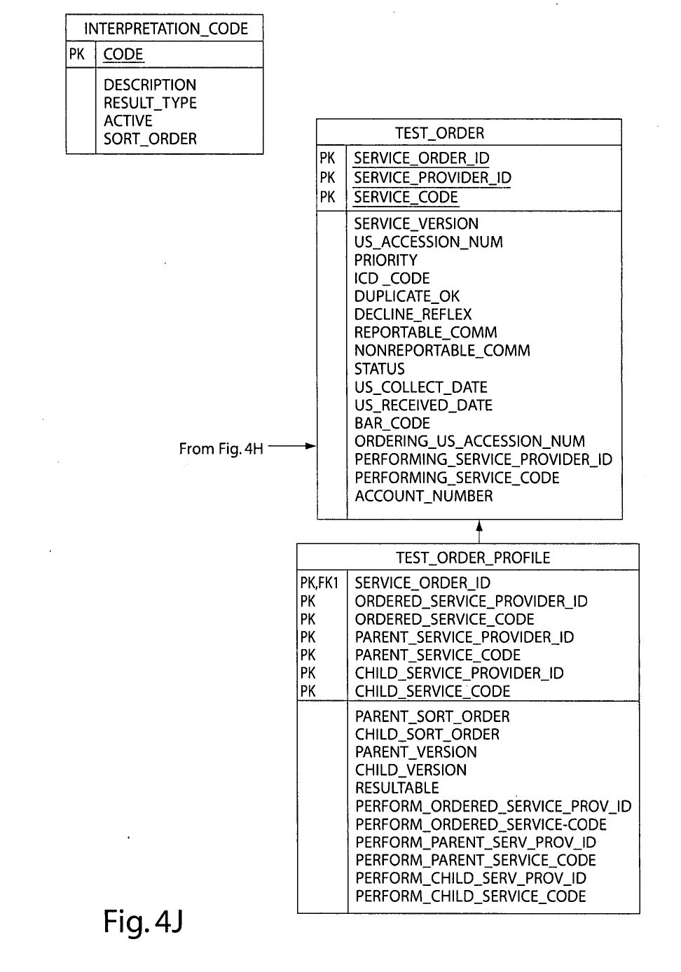

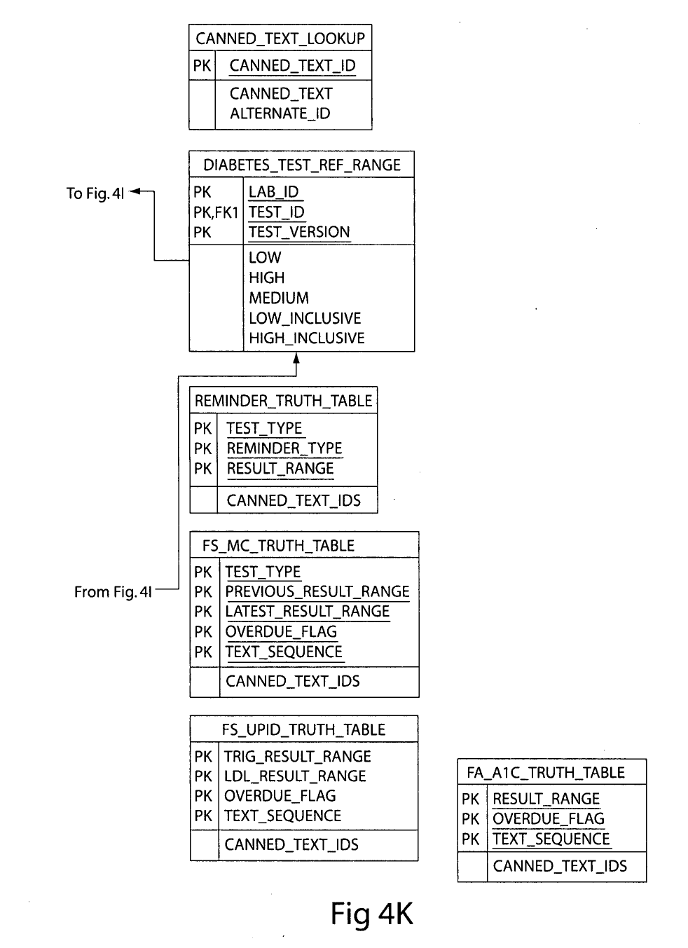

The Operations Database is schematically shown in FIG. 4. The operations database can be further segmented into three domains: (a) Patient and provider demographics, including provider, practice and patient demographic information and relationships among these entities, and current and historical patient and provider status change information; (b) Lab results, including test codes, values, dates, accession numbers, cross reference of each lab's local test code information into registry's specific test code information, lab result range and lab overdue information; and (c) Monitoring, Reporting and Data import operations, including web application login information, site specific data import configuration and audit trail information, data import filtering information, error logs, report creation audit trail and control limits for operational metrics. The operations database can be made secure with password protection, with limited access, for example to a Project Director or IS Support. In a certain embodiment the database backup to tape is performed on a server nightly.

The Practice Database (Access Format) serves as a front end to the operations database for administrative functions. The practice database contents include information about the physician and practice such as contact information for potential and study practices, recruitment and study status etc., and information about the patient; the practice database is linked to the operation database for viewing patient level data and for entry of status and address changes. Security is provided by directory level security limited access to shared files and the practice database and password protection, with limited access, for example to Project Director and IS Support and Operations staff. In a certain embodiment the database backup to tape is performed on a server nightly. The practice database can also function to provide patient status and address changes and/or patient interview scheduling.

The Web Data Entry Interface is used for entry of lab data that are collected in the individual practices with point of care lab testing devices. These results are not routinely interfaced with the participating lab information systems. The Web Data Entry Interface contains result entries: lab results are added directly into the operations database and/or order inquiry: queried or updated existing labs previously entered from web interface. Security for the The Web Data Entry Interface is provided by password protected access, for example access is limited to Operations Staff. In certain embodiments of the invention the The Web Data Entry Interface functions to allow for data entry of laboratory results, order inquiries, and/or updates of order inquiries.

In a certain aspect of the invention a Research Database is provided that is connected to the operations database. These databases are for research data and are not part of routine registry database operations. The research (STATA format) databases will be populated from queries of the operations database and have any identifying information stripped.

As will be appreciated by one of skill in the art, the present invention may be embodied as a method, data processing system, or computer program product. Accordingly, the present invention may take the form of an entirely hardware embodiment, an entirely software embodiment or an embodiment combining software and hardware aspects. Furthermore, the present invention may take the form of a computer program product on a computer-readable storage medium having computer-readable program code means embodied in the medium. Any suitable computer readable medium may be utilized including hard disks, CD-ROMs, optical storage devices, or magnetic storage devices.

As used herein an “automated electronic system” is any electronic system that is capable of automatically performing the methods of the invention, including a computer, a processor, or any machine or apparatus capable of transferring or collecting data, performing data interpretation and generation of decision support reports. As used here in “a storage device” is any device capable of storing data, preferable a mass storage device, such as magnetic disk, an optical disk or a tape drive. As used here in “a processor for automatically retrieving” and “processor for sending” refers to a central processing unit configured to automatically retrieve data and send data and/or reports, respectively. The processors may be a single processor configured to handle both functions or they may be separate processors.

The present invention is described herein with reference to flowchart illustrations of methods, apparatus (systems) and computer program products according to embodiments of the invention. It will be understood that each block of the flowchart illustrations, and combinations of blocks in the flowchart illustrations, can be implemented by computer program instructions. As used herein “computer readable code” refers to a computer program configured to perform the methods of the invention. Therefore, computer readable code for generating and maintaining a patient registry database is a computer program that can be used to generate and maintain a database. Computer readable code for retrieving clinical information from a remote data site is a computer program that can be used to retrieve clinical information from a remote data site. Computer readable code for interpreting the clinical information is a computer program that can be used to interpret clinical information. Computer readable code for reporting the interpretation of the clinical information is a computer program that can be used to report the interpretation of the clinical information. These computer program instructions may be loaded onto a general purpose computer, special purpose computer, or other programmable data processing apparatus to produce a machine, such that the instructions which execute on the computer or other programmable data processing apparatus create means for implementing the functions specified in the flowchart block or blocks.

These computer program instructions may also be stored in a computer-usable memory that can direct a computer or other programmable data processing apparatus to function in a particular manner, such that the instructions stored in the computer-usable memory produce an article of manufacture including instruction means which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks.

Accordingly, blocks of the flowchart illustrations support combinations of means for performing the specified functions, combinations of steps for performing the specified functions and program instruction means for performing the specified functions. It will also be understood that each block of the flowchart illustrations, and combinations of blocks in the flowchart illustrations, can be implemented by special purpose hardware-based computer systems which perform the specified functions or steps, or combinations of special purpose hardware and computer instructions.

Computer program for implementing the present invention may be written in various object-oriented programming languages, such as Delphi and Java.RTM. However, it is understood that other object oriented programming languages, such as C++ and Smalltalk, as well as conventional programming languages, such as FORTRAN or COBOL, could be utilized without departing from the spirit and intent of the present invention.

As described herein, patient refers to a patient afflicted with a chronic disorder. As used herein “chronic disorder” is any illnesses that is prolonged, does not resolve spontaneously, and are rarely cured completely and therefore it requires long term medical care, monitoring and management. In certain aspects of the invention the chronic disorder is being managed by a primary care practice. In a preferred embodiment of the invention the patient is a diabetic patient.

The term “diabetic patient” refers to a patient that is affected by, or at risk of developing, diabetes and/or any of a group of related disorders in which there is a defect in the regulation of circulatory and/or intracellular glucose (sugar) levels. Diabetic patients include subjects with abnormally high levels of blood sugar (hyperglycemia) or abnormally low levels of blood sugar (hypoglycemia).

Diabetes is a highly debilitating and increasingly common disorder that is typically associated with impaired insulin signaling. There are 18.2 million people in the United States, or 6.3% of the population, who have diabetes. The major types of diabetes are:

Type 1 diabetes results from the body's impairment of insulin production due to loss of pancreatic beta cells. It is estimated that 5-10% of Americans who are diagnosed with diabetes have type 1 diabetes. Type 1 diabetes is usually diagnosed in children and young adults, and was previously known as juvenile diabetes. Conditions associated with type 1 diabetes include hyperglycemia, hypoglycemia, ketoacidosis and celiac disease. Some complications of type 1 diabetes include: heart disease (cardiovascular disease), blindness (retinopathy), nerve damage (neuropathy), and kidney damage (nephropathy).

Type 2 diabetes results from insulin resistance (a condition in which the body fails to properly use insulin—cellular sensitivity to circulating insulin is impaired), combined with relative insulin deficiency. Approximately 90-95% (17 million) of Americans who are diagnosed with diabetes have type 2 diabetes. Type 2 diabetes increases the risk for many serious complications including heart disease (cardiovascular disease), blindness (retinopathy), nerve damage (neuropathy), and kidney damage (nephropathy).

Pre-diabetes is a condition that occurs when a subject's blood glucose levels are higher than normal but not high enough for a diagnosis of type 2 diabetes. It is estimated that before subjects develop type 2 diabetes, they almost always have “pre-diabetes”—blood glucose levels that are higher than normal but not yet high enough to be diagnosed as diabetes. At least 20.1 million people in the United States (21.1% of the population), ages 40 to 74, have pre-diabetes. Recent research has shown that some long-term damage to the body, especially the heart and circulatory system, may already be occurring during pre-diabetes.

There are tests routinely used by those of ordinary skill in the art to establish if a subject is a “diabetic subject”. Two different tests that can be used to determine whether a subject is a “diabetic subject” are: the fasting plasma glucose test (FPG) or the oral glucose tolerance test (OGTT). The blood glucose levels measured after these tests can be used to determine whether a subject has a normal metabolism, or whether a subject is a “diabetic subject,” in other words whether a subject has pre-diabetes or diabetes. If the blood glucose level is abnormal following the FPG, the subject has impaired fasting glucose (IFG); if the blood glucose level is abnormal following the OGTT, the subject has impaired glucose tolerance (IGT). In the FPG test, the subject's blood glucose is measured first thing in the morning before eating. In the OGTT, the subject's blood glucose is tested after fasting and again 2 hours after drinking a glucose-rich drink.

Normal fasting blood glucose is below 100 mg/dl. A subject with pre-diabetes has a fasting blood glucose level between 100 and 125 mg/dl. If the blood glucose level rises to 126 mg/dl or above, the subject has diabetes. In the OGTT, the subject's blood glucose is measured after a fast and 2 hours after drinking a glucose-rich beverage. Normal blood glucose is below 140 mg/dl 2 hours after the drink. In pre-diabetes, the 2-hour blood glucose is 140 to 199 mg/dl. If the 2-hour blood glucose rises to 200 mg/dl or above, the subject has diabetes.

According to the invention, a subject at risk of developing diabetes or a related disorder is a subject that is predisposed to such the disease or disorder due to genetic or other risk factors. While diabetes and pre-diabetes occur in subjects of all ages and races, some groups have a higher risk for developing the disease than others. Diabetes is more common in African Americans, Latinos, Native Americans, and Asian Americans/Pacific Islanders, as well as the overweight and aged population. Most people diagnosed with type 2 diabetes are overweight. A healthy weight is determined by your body mass index (BMI), which can be calculated based on subjects height and weight. Overweight is defined as a BMI greater than/equal to 25; obesity is defined as a BMI greater than/equal to 30. Overweight and obese subjects are at increased risk for developing pre-diabetes and diabetes. A family history of diabetes is also a risk factor. Age can also be a risk factor. In some embodiments, a subject at risk is identified as a subject having one or more of these risk factors. These risk factors can be assessed using risk factor tests known in the art.

According to the invention, the term “treatment” includes managing a diabetic subject's glucose levels. Treatment also encompasses prophylaxis to prevent or slow the development of diabetes, and/or the onset of certain symptoms associated with diabetes in a subject with, or at risk of developing, diabetes or a related disorder. For example, in the case of a diabetic subject with pre-diabetes, treatment means decreasing the likelihood that the subject will develop Type 2 diabetes.

Hyperglycemia is one of the cardinal lesions in diabetes, but because blood sugars fluctuate so widely over time, they are poor markers of long-term control. However, prolonged exposure to elevated glucose levels in the blood causes a chemical change in the normal hemoglobin found in red cells. Glycated hemoglobin (also called hemoglobin AlC or HbAlc) is found to make up less than about 6% of hemoglobin in non-diabetic patients. The HbAlc level is correlated to the average degree of hyperglycemia over the previous six weeks. The desirable target for diabetics is less than 7%, with lower numbers associated with fewer long-term diabetic complications such as nephropathy, neuropathy, vascular disease, retinopathy, etc. The 1998 United Kingdom Prospective Diabetes Study (UKPDS) established that rates of retinopathy, nephropathy, and neuropathy are reduced in Type II diabetes with intensive therapy, which achieved a median HbAlc level of 7.0%. There is a continuous relationship between glycemic control and the risks of microvascular complications, such that for every percentage point decrease in HbAlc, there is a 35% reduction in the risk of complications. Therefore, the guidelines call for HbAlc to be measured every six months in diabetics thought to be in good control and every three months in newly diagnosed or uncontrolled diabetics.

Diabetic coronary heart disease can be prevented by tight control of serum lipids. The best marker of hyperlipidemia in diabetes is controversial, but most guidelines recommend measuring Low Density Lipoprotein Cholesterol (LDL) every year and using diet, exercise and medications to maintain it below 130 mg/dl. The threshold is lowered to 100 mg/dl for patients with other coronary risk factors.

Stroke and other vascular complications can be reduced in diabetics by maintaining blood pressure in normal ranges. Most guidelines advise using diet, exercise and medications to maintain systolic pressure below about 135 mmHg, and diastolic below about 85 mmHg.

Renal failure can be averted or delayed by early use of angiotensin converting enzyme (ACE) inhibitor drugs at the first sign of nephropathy. One of the earliest signs of diabetic nephropathy is leakage of the blood protein albumin into the urine in small amounts. Microalbuminuria is measured by calculating the ratio of urine protein concentration to the serum creatinine level. Although there is some controversy about the effects of ACE inhibitors, most guidelines advise that if the M:C ratio is above 30 mg/g, ACE inhibitor therapy should be considered.

Thus, according to the invention diabetic patients can be part of the CDSS. It is recommended that such patients undergo regular testing for AlC, serum lipid tests, urinary microalbumin to creatinine ratio (MCR), and/or serum creatinine. The clinical information obtained by the test can be used by the methods and systems of the invention for clinical decision support and management of the patient's diabetic condition of the patient by the healthcare providers. As described herein there are numerous advantages that an automated decision support system can provide in management of chronic disorders to healthcare providers, primary care practices and patients, especially in remote areas.

In one aspect of the invention, the patient has a cholesterol related disorder. Cholesterol is a lipid that plays a role in the production of cell membranes, some hormones, and vitamin D. High blood cholesterol is a significant risk factor in heart disease. Lowering blood cholesterol through increased physical activity, weight loss, smoking cessation, and proper diet lowers that risk. However, blood cholesterol is very specific to each individual and, for that reason, a full lipid profile is an important part of a medical history and important clinical information for a physician to have. Cholesterol is transported in the blood stream in the form of lipoproteins. The two most commonly known lipoproteins are low-density lipoproteins (LDL) and high-density lipoproteins (HDL). In general, healthy levels are as follows: LDL—less than 130 milligrams; HDL—less than 35 milligrams, and total cholesterol level below 200 is considered desirable. Triglycerides are another class of fat found in the bloodstream. Elevated triglyceride levels may be caused by medical conditions such as diabetes, hypothyroidism, kidney disease, or liver disease. Dietary causes of elevated triglyceride levels may include obesity and high intakes of fat, alcohol, and concentrated sweets. A healthy triglyceride level is less than 150 mg. According to aspects of the invention, the LDL, HDL and triglyceride tests can be used as clinical information in a CDSS for the management of cholesterol related disorders.

In one aspect of the invention the patient has a thyroid related disorder. The thyroid is a gland that controls key functions of your body. Disease of the thyroid gland can affect nearly every organ in your body and harm health. Thyroid disease is eight times more likely to occur in women than in men. In some women it occurs during or after pregnancy. The thyroid gland makes, stores, and releases two hormones—T4 (thyroxine) and T3 (tri-iodothyronine) that control metabolic rates. The thyroid gland is controlled by the pituitary gland (a gland in the brain). The pituitary gland makes thyroid-stimulating hormone (TSH). If there is not enough thyroid hormone in the bloodstream, the body's metabolism slows down—hypothyroidism (under active thyroid). If there is too much thyroid hormone, the metabolism speeds up—hyperthyroidism (overactive thyroid). Thyroid disease is diagnosed by clinical information such as symptoms, examination and tests. Tests include: blood tests, ultrasound exam (during pregnancy), thyroid scan etc.

In one aspect of the invention the patient is afflicted with hepatitis. Hepatitis A is a serious liver disease caused by the hepatitis A virus (HAV). HAV is found in the feces of people with hepatitis A and is usually spread by close personal contact (including sex or sharing a household). It can also be spread by eating food or drinking water contaminated with HAV. There is no treatment for hepatitis A.

HBV and/or HBC is found in blood and certain body fluids. It is spread when blood or body fluid from an infected person enters the body of a person who is not immune. HBV is spread through having unprotected sex with an infected person, sharing needles or “works” when “shooting” drugs, needlesticks or sharps exposures on the job, or from an infected mother to her baby during birth. Exposure to infected blood in any situation can be a risk for transmission. Persons with chronic HBV and/or HBC infection should have a medical evaluation for liver disease every 6-12 months. Several antiviral medications are currently licensed for the treatment of persons with chronic hepatitis B. The clinical information useful for managing a patient afflicted with hepatitis comprises blood test for hepatitis antigens and/or antibodies, blood tests for alanine aminotransferase (ALT) and aspartate aminotransferase (AST) levels (both are enzymes released when liver cells are injured or die), and liver biopsy.

In one aspect of the invention the patient is a cancer patient. Cancer refers to any disorder of various malignant neoplasms characterized by the proliferation of anaplastic cells that tend to invade surrounding tissue and metastasize to new body sites and the pathological conditions characterized by such growths. Accordingly, the methods of the invention are useful in the management of the treatment of cancer. Cancers include but are not limited to: biliary tract cancer; bladder cancer; breast cancer; brain cancer including glioblastomas and medulloblastomas; cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS-associated leukemias and adult T-cell leukemia lymphoma; intraepithelial neoplasms including Bowen's disease and Paget's disease; liver cancer; lung cancer including small cell lung cancer and non-small cell lung cancer; lymphomas including Hodgkin's disease and lymphocytic lymphomas; neuroblastomas; oral cancer including squamous cell carcinoma; esophageal cancer; osteosarcomas; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, synovial sarcoma and osteosarcoma; skin cancer including melanomas, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilms tumor. A patient is preferably a patient diagnosed with cancer. A patient can be diagnosed with cancer using any recognized diagnostic indicator including, but not limited to, physical symptoms, molecular markers, or imaging methods. A patient can also be a subject at risk of developing cancer; a patient that has been exposed to a carcinogen or other toxin, a patient with one or more genetic predispositions for cancer, a patient with symptoms of early cancer, or a patient that has been treated for cancer and is at risk of cancer recurrence or metastasis.

Clinical information for a cancer patient includes the results of laboratory tests, imaging or medical procedure directed towards the specific cancer that one of ordinary skill in the art can readily identify. The list of appropriate sources of clinical information for cancer includes but it is not limited to: CT scan, MRI scan, ultrasound scan, bone scan, PET Scan, bone marrow test, barium X-ray, endoscopy, lymphangiogram, IVU (Intravenous urogram) or IVP (IV pyelogram), lumbar puncture, cystoscopy, immunological tests (anti-malignin antibody screen), and cancer marker tests.

### EXAMPLES

### Example 1

### The Vermont Diabetes Information System (VDIS) Preliminary Study

Methods. VDIS is a decision support and reminder system for primary care practices and their patients with diabetes. It involves some of the principles of quality improvement of Donabedian (Donabedian A., Vol. 1, Ann Arbor: Health Administration Press, 1980) and the Chronic Care Model of illness management (Bodenheimer T., et al., JAMA 2002; 288:1775-79; Bodenheimer T., et al., JAMA 2002; 288:1909-14). The Chronic Care Model emphasizes the importance of bringing together for an ideal clinical encounter a prepared, proactive health care team and an informed, active patient. Chronic disease registries are a central aspect of this model. While other implementations of the chronic care model require substantial investment by the practice and major changes in the providers' usual activities, VDIS was designed to require a minimum of effort and no new financial resources on the part of the providers.

Technical description of VDIS. There are five components that can be involved in VDIS: 1) use of the Chronic Care Model as an organizing framework; 2) daily data feeds from otherwise independent laboratories; 3) automatic test interpretation using algorithms based on consensus guidelines; 4) use of fax and mail to report to providers and patients not easily reached by electronic networks; and 5) report formats that are accessible and useful to patients and providers.

A primary function of the system is to collect pertinent clinical information and to provide accurate and timely flow sheets, reminders, and alerts to physicians and their patients with diabetes. Secondly, the system generates summary population reports for physicians regarding their roster of diabetic patients. The intended effects of the interventions are outlined in Table 1.

TABLE 1
Anticipated effects of VDIS interventions

| | |
|---|---|
| Intervention | Anticipated effect |

Directed to the practice and primary care provider

| | |
|---|---|
| Faxed lab flow sheets with recent | Provide decision support and |
| test results and guideline-based | stimulate appropriate action by |
| recommendations. | provider. |
| Faxed reminders when patients | Stimulate follow-up of patients |
| are overdue for recommended | who are lost to follow up or |
| laboratory testing. | otherwise overdue. |
| Mailed quarterly population | Provide the provider a population- |
| reports with report cards of | based view of his or her entire |
| individual provider performance | diabetes patient roster for |
| and lists of patients sorted by | targeted case management. Allow |
| degree of control based on | provider to keep roster of patients |
| laboratory tests. | up to date. Peer comparison may |
| | motivate a practice to modify |
| | office processes for chronic |
| | illness management. |

Directed to the patient

| | |
|---|---|
| Mailed alerts when a laboratory | Engage and activate patients to |
| test result is above guideline- | know and understand the goals of |
| based threshold | therapy and to be prepared for |
| | interaction with the provider. |
| Mailed reminders when patients | Remind patient to schedule follow |
| are overdue for recommended | up testing or an office visit. |
| laboratory testing. | |

Data loading. For each participating practice, an initial list of patients is developed by the laboratory, based on all patients who have had an AlC test performed in the previous two years. This list is verified by the primary care provider (PCP) to determine the eligibility of each patient. Once the PCP has verified the list, the patient demographic data are loaded into a custom Oracle data repository. Subsequently, the laboratory prepares a two-year historical report of laboratory results for those patients and this information is loaded into the database for seeding of flow sheets, reminders and alerts. The laboratory results that are pertinent to management of most patients with diabetes, and that are the subject of guideline recommendations, are the AlC, serum lipid tests, urinary microalbumin to creatinine ratio (MCR) and the serum creatinine.

Nightly data collection and processing. The collection of the laboratory data in a timely manner is part of the creation and distribution of the flow sheets and medical reports. A nightly program automatically reports that day's AlC, lipid, microalbumin and creatinine results on the population of identified subjects. This file is transferred using file transfer protocol (FTP) and a variety of secure connection methods. Most of the connections are done via branch-to-branch virtual private network (VPN) connections over the Internet or private leased data lines. These daily report files are then processed into the registry database. The system also allows manual data input via a secure Internet forms software function. The software accepts the medical record number and test results and processes them into the registry. This function allows practices performing point of care testing in the office to directly enter test results.

Report triggering. The report generator function may run automatically each night after results are received. Any laboratory result for AlC, LDL, creatinine or MCR triggers the creation and faxing to the PCP of a flow sheet displaying the current results, the previous four results in the database (to display trends), and decision support recommendations based on published guidelines (Vermont Program for Quality in Health Care, 2004; ADA, Diabetes Care 2004; 27(Suppl. 1):515-35). If a result is above a threshold level, an alert letter is electronically sent to a mail and production service for mailing to the patient. If a patient is overdue for a laboratory test, an alert fax is sent to the provider, and a letter is mailed to the patient to remind them both of the recommended testing. None of the VDIS output is part of the permanent medical record and does not require filing in the chart. The laboratories continue to send their routine reports to the practices. The thresholds for designating a result to be high were taken from a Vermont guideline (Vermont Program for Quality in Health Care, 2004) based on the American Diabetes Association Clinical Practice Recommendations (ADA, Diabetes Care 2004; 27(Suppl. 1):515-35) for a change in therapy (AlC . 8%; LDL. 130 mg/dL;

MCR. 300 mg/Mg). While the guidelines are well understood and published these algorithms required significant additional logic to create an operational system acceptable to busy clinical providers and to patients. Effective algorithms for “Grace Periods” were developed in order to avoid reminding a patient about a required test when that patient may have a test scheduled in the coming weeks. Effective algorithms for “Refractory Periods” were developed to avoid re-reminding a patient too frequently about overdue tests. Clinical examples are included herein and in the Appendices. Grace and Refractory periods are configurable in the VDIS system.

An AlC may be considered to be overdue if the previous AlC is more than six months old, or if the previous AlC is 7.0% or greater and more than three months old. A one month grace period is allowed, so a patient reminder letter is not generated until seven or four months have elapsed. A six to 12 month overdue period (plus the one month grace period) is applied to LDL and MCR depending on the result range. Since microalbumin testing is often stopped after the development of proteinuria (and appropriate therapy with medications directed at the renin-angiotensin system), MCR reminders are suppressed once the patient has microalbuminuria.

Quarterly population reports are intended to provide the PCP with a population-based view of his or her roster of diabetic patients. PCPs are encouraged to use the roster for identification of patients who are off guideline or lost to follow-up. The population report also contains comparisons of individual PCP performance with the performance of the entire study population for both on-target and on-time with guideline-based goals. It is also possible to include a top 10% performance measure, the achievable benchmark of care (Kiefe C. I. et al., JAMA 2001; 285:2871-79; Weissman N. W., et al., J. Eval. Clin. Pract. 1999; 5:269-81).

Practices and study subjects. Laboratories were recruited for VDIS through the Northeast Community Laboratory Alliance and personal communication with laboratory directors and hospital administrators. Ten of the 14 hospital-based laboratories in Vermont as well as four in nearby New York and another in nearby New Hampshire have joined the study. Technical personnel from each laboratory work with the investigators to create a secure connection for the daily transmission of laboratory results. To be eligible, an internal medicine or family medicine practice must: 1) use one of the participating laboratories; 2) care for patients with diabetes; 3) be able to receive faxes; and 4) provide consent. Practices using point of care testing devices for a small proportion of their testing were invited to participate if we were able to arrange for an efficient method of data acquisition. This was accomplished by daily fax of point of care test results to the VDIS office and web-based data entry into the system by VDIS staff. Some of the largest practices in the state, most notably the faculty practices of the University of Vermont, were not eligible to participate because they were involved in pilot work for this study. Over a hundred practices were identified and contacted that were potentially eligible for participation in the study from the customer lists of the participating labs and by personal communication with providers around the state. Once a practice was enrolled, a list of all patients with a test for AlC in the previous two years was generated by the laboratory. These lists were reviewed by each PCP to identify those patients who met the following eligibility criteria: 1) diabetes type 1 or type 2; 2) age 18 or older; 3) under the care of that PCP for diabetes; and 4) not suffering from cognitive impairment that would prevent understanding reminders, per the judgment of the PCP. Any conflicts were resolved by discussion with the PCP offices. If a patient was receiving the majority of diabetes care from an endocrinologist or other provider, they were not included on the final PCP roster. It was not distinguished between Type 1 and Type 2 diabetes because the ADA guidelines do not differ substantially regarding testing frequency or therapeutic goals, and because it is often unclear clinically which type of diabetes is present. If a new patient with diabetes is encountered in the course of the study, they may be added to the system for clinical purposes, but are not part of the study population.

A practice is affiliated with a laboratory. In the study it was desired to ensure that no laboratory had a gross preponderance of active or control practices. Each laboratory represented a stratum in a stratified and blocked randomization scheme. A series of numbered, sealed, opaque envelopes were created for each stratum (each laboratory). The envelopes contained a card indicating either CONTROL or ACTIVE condition. Blocks of four or six envelopes were filled with balanced numbers of ACTIVE and CONTROL cards, sealed, and shuffled thoroughly within blocks. In that way, each stratum was likely to have an approximately equal number of active and control practices. After each practice was recruited and consented, the next envelope in their laboratory stratum's series was opened to determine the assignment for that practice. The practice was chosen as the unit of randomization because of the sharing of patients and systems of care among PCPs in the same office. Intervention practices receive the VDIS intervention while the control practices have patient data collected behind the scenes, and otherwise continue with usual care.

Consent process and privacy issues. Decision support services (such as the information systems, registry functions, reminders, and reports of VDIS) are clinical quality improvement activities that require personal health information as defined and protected under the Health Insurance Portability and Accountability Act (HIPAA).

Providers may generally conduct such activities without a specific consent from the patient, although certain restrictions apply such as protection of patient confidentiality. To ensure that the registry data could not be accessed by others, VDIS is structured as a regional quality improvement initiative under the direction and supervision of the Vermont Program for Quality in Health Care (VPQHC), a state chartered peer-review organization.

Although not required by law, we employ a passive (“opt-out”) consent process for inviting patients into the study. After the patient is identified, but before any services are initiated, we mail a letter to the patient on behalf of the PCP. The letter describes the study and invites the patient to participate. It requests that the patient call the provider or a toll-free number at the University, if they prefer not to participate. All laboratory data for these patients are removed from the database.

The PCPs are also considered subjects of the research. Therefore, each participating provider signs an informed consent agreement.

VDIS survey. One advantage of the design of VDIS is that, once the connection to the lab is made, the cost of acquisition of lab data is negligible. One disadvantage is that these data are limited to laboratory results, sex and date of birth. In order to obtain a deeper understanding of the study population and the impact of the intervention, we designed a survey targeted at a randomly selected 10% subsample of patient subjects.

Practice rosters are randomly sorted and patients invited by phone to participate in an in-home interview consisting of a questionnaire, measurement of height using a portable stadiometer (SECA, Inc.), weight (LB Dial Scale HAP200KD-41, Healthometer, Inc.), blood pressure (Omron automated sphygmomanometer, Model HEM-711) and administration of a test of health literacy. Blood pressure is obtained in the seated position in the left arm (unless contraindicated), using the cuff size recommended by the manufacturer. Three readings are obtained at five-minute intervals and are averaged for the final result. The research assistant reviews questionnaires for completeness at the time of the interview. Patients are reimbursed $20 for their time. Patients who are enrolled in the substudy provide full written informed consent before they are interviewed. Table 2 lists the variables included in the VDIS study, including those in the survey.

TABLE 2
Study variables in the VDIS trial

| | |
|---|---|
| Dimension | Variables |

Laboratory data

| | |
|---|---|
| Glycemic control | A1C |
| Lipid control | Total cholesterol, triglyceride, high |
| | density lipoprotein, low density |
| | lipoprotein |
| Renal function | Creatinine, microalbumin:creatinine |
| | ratio |
| Demography | Date of birth, sex |

Physical examination and direct observation

| | |
|---|---|
| Obesity | Height, weight, body mass index |
| Hypertension | Blood pressure |
| Heart Rate | Pulse |
| Functional Health Literacy | Short test of functional health |
| | literacy in adults |
| Medications | Medication list with name, dose, |
| | frequency of all prescription, over-the- |
| | counter, herbal or supplement preparations |
| | used in the last month |

Self report

| | |
|---|---|
| Demography | Income, education, marital status, |
| | race/ethnicity, health insurance |
| Health habits | Smoking, drinking, exercise habits |
| Functional status | Medical Outcomes Trust SF-12 |
| Diabetes-related quality | The Audit of Diabetes-Dependant |
| of life | Quality of Life |
| Diabetes self care | Summary of Diabetes Self Care |
| | Activities Measure |
| Health care utilization | Self-report of visits to primary care, |
| | emergency room, endocrinology, |
| | ophthalmology, diabetes educator, |
| | dietician |
| Complication status | Self-report of diabetes complications |
| Comorbidity | Self Administered Comorbidity |
| | Questionnaire |
| Patient satisfaction | Primary Care Assessment Survey |
| Diabetes utility | Paper Standard Gamble |
| Depression | Patient Health Questionnaire-9 |

The Medical Outcomes Trust SF-12 is a widely used, validated instrument for assessment of general (rather than disease-specific) functional status (Ware J. E. et al., Quality Metric Inc., 2002). Summary scales covering mental and physical functioning are calculated: the physical component summary and the mental component summary.

The Audit of Diabetes-Dependant Quality of Life is an 18-item questionnaire regarding the impact of diabetes on specific aspects of a person's life with patient weighting of the impact of each domain (Bradley C. et al., Qual. Life Res. 1999; 8:79-91; Bradley C., et al., Diabetes Metab. Res. Rev. 2002; 18(Supp. 3): S64-69). Another approach to health related quality of life is to measure the subject's quantitative preference for their current health. This measure, called “utility”, is widely used in cost-effectiveness analyses and other economic studies. The Paper Standard Gamble is a one page assessment of patient utility that has been validated for use in postal surveys (Littenberg B., et al., Med. Decis. Making 2003; 23:480-88).

The Self-Administered Comorbidity Questionnaire is a modification of the widely used Charlson Index. It uses patient interview or questionnaire rather than chart abstraction for assessment of comorbidity and has excellent agreement with the chart-based Charlson Index (Katz J. N. et al., Med. Care 1996; 34:73-84; Sangha O., et al., Arthritis Rheum. 2003; 49:156-63).

The Short Test of Functional Health Literacy in Adults is a seven-minute timed instrument that measures the ability to read health-related material (Baker D. W. et al., Patient Educ. Couns. 1999;38:33-42; Parker R. M. et al., J. Gen. Intern. Med. 1995; 10:537-41).

The Primary Care Assessment Survey is a validated, 51-item patient-completed questionnaire designed to measure the essential elements of primary care. It measures seven characteristics of primary care through 11 summary scales: accessibility, continuity, comprehensiveness, integration of care, clinical interaction, interpersonal treatment, and trust (Safran D. G. et al., Med. Care 1998; 36:728-39).

The Patient Health Questionnaire-9 is a brief self report instrument that quantifies the presence and degree of mental depression (Kroenke K. et al., J. Gen. Intern. Med. 2001; 16:606-13).

Statistical approach. This is a two-arm randomized trial with clustering by practice. Our primary null hypothesis is that there will be no difference between the intervention and control groups in mean AlC level at study's end. Secondary analyses will focus on group differences in lipids, creatinine, proportion on guideline, and proportion adhering to specific guideline components (overdue for specific tests or out of range for specific tests). We will use a general linear mixed model for outcomes with normally distributed residual errors, or a generalized linear mixed model for outcomes with binomial distribution for residual errors (Littell R. C. et al., SAS System for Mixed Models, Cary, NC:SAS Institute, Inc., 1996). The primary analysis will include all participants and use final hemoglobin AlC as the dependent variable. Independent variables will be dichotomous variables representing randomization status (1¼ active; 0¼ control) and patient sex, and continuous variables representing hemoglobin AlC at baseline and patient age. Since the unit of randomization is the practice, we will adjust all standard errors for clustering on practice. Clustering reduces statistical power in proportion to the degree that subjects within each cluster are similar. To account for this, we modeled sample size using the methods of Donner and others (Koepsell T. H. et al., Ann. Rev. PubL. Health 1992; 13:31-57; Donner A., et al., Am. J. Epidemiol. 1981; 114:906-14; Donner A. et al., Am. J. Public Health 2004; 94:416-22), which require an estimate of the intraclass (or within practice) correlation coefficient to use in a variance inflation factor. Initial data from VDIS indicate a standard deviation of AlC of 1.4% and an intra-class correlation of 0.02. There are, on average, 125 eligible subjects per practice. Using alpha ¼ 0.05 and a power of 80%, we require 20 randomized practices (10 per arm) to detect a difference between control and active groups of 0.3%. To detect a difference of only 0.2% requires 44 randomized practices per arm. Currently, 55 practices have been activated and another 17 are in the process of coming into the system.

Results. The data is based on the 10 hospitals, 55 practices, 121 primary care providers and 7348 patients who are currently active in the VIDS system. The baseline characteristics of the patient population are shown in Table 3. The demographic characteristics match the population of Vermont (US Census 2000). Two hundred and seven invited patients have declined participation. The refusal rate is 207/7555 or 2.7%. Patients cite a variety of reasons including “feeling too ill”, “too old”, concerns regarding privacy and sharing of lab data and not identifying oneself as a diabetic. The number of primary care providers per practice averages 2.1 with a range of 1-6. Of the PCPs, 93 are physicians, 13 are nurse practitioners and 15 are physician assistants. The mean PCP panel size is 59 patients with a range of 1-201. The mean practice panel size is 125 patients with a range of 12-353.

At an average follow-up of 12 months improvements were found in test ordering frequency for AlC, lipids, and urinary microalbumin.

TABLE 3
Baseline characteristics of the VDIS patient population

| | |
|---|---|
| Characteristic | Result |

Registry data (n = 7348)

| Age in years, mean (range) | 62.9 | (18-99) |
|---|---|---|
| Female | 51% | |
| A1C, mean (SD) | 7.1 | (1.4) |

| | |
|---|---|
| A1C in excellent control (≦7%) | 60% |
| A1C on time (within 3 months if A1C <7%; 6 months | 49% |
| if A1C >7%) | |
| Lipids in control (LDL <100 mg/dL; | 45% |
| trigylceride <400 mg/dL | |
| Lipids on time (within 12 months) | 67% |
| Microalbuminuria absent (<30 mg/g) | 69% |
| Microalbumin test on time (within 12 months) | 23% |

Survey data (n = 746)

| Race (% white) | 97% | |
|---|---|---|
| Education (% some college) | 41% | |
| Smoking (% current smokers) | 15% | |
| Income (<$30 000/y) | 56% | |
| Body mass index (SD) | 33.7 | (7.8) |
| Excellent blood pressure control (<=130/80 mm Hg) | 25% | |
| Poor blood pressure control (>140/90 mm Hg) | 49% | |
| SF-12 Physical component summary, mean (SD) | 41.8 | (12.3) |
| SF-12 Mental component summary, means (SD) | 50.2 | (10.5) |
| Duration of diabetes in years, mean (range) | 10.9 | (0.3-63) |
| Number of comorbid conditions, mean (range) | 1.8 | (0-13) |

SD = standard deviation; LDL = low density lipoprotein cholesterol.

### VIDS Operations Overview—Technical Summary

A. High level overview of VDIS

The Vermont Diabetes Information System is a specific instance of a decision support system (DSS) that is targeted at patients with diabetes and the physicians and other health care providers who are caring for them in the primary care setting. In brief, lab data are uploaded from participating clinical laboratories to the VDIS data registry on a regular, i.e. nightly basis. Reminders, alerts and population reports are then sent to patients and providers, prompting guideline-based care. In order for patients to be included in the study it was determined that they should be cared for in a participating practice, that practice should be using a participating lab, or doing in-office point of care testing in such a way that lab results can be transmitted to VDIS on a timely basis.

The details of the database structure and the procedures for the enrollment of labs, practices and patients are included in this Example. Some of the functions are specific to the research aspects of the VDIS project, and others to the general operation of the system.

B. Summary of Operation Related Figures:

FIG. 2 depicts the sequence of steps involved in the initial configuration of laboratories, practices and patients and the loading of lab data in VDIS.

FIG. 3 depicts the sequence of steps involved in the steady state daily operations of the CDSS and specifically VDIS.

FIG. 4 depicts a schema of the VDIS database.

C. Database Summaries:

1. VDIS database—the operations database:

The database is segmented into three domains:

1. Patient and provider demographics, including: Provider, practice and patient demographic information and relationships among these entities and Current and historical patient and provider status change information

2. Lab results: Lab results (test codes, values, dates, accession numbers), Cross reference of each lab's local test code information into VDIS specific test, code information and Lab result range and lab overdue information.

3. Monitoring, Reporting and Data import operations: VDIS web application login information, Site specific data import configuration and audit trail information, Data import filtering information, Error logs, Report creation audit trail, Control limits for operational metrics, Security, Password protection, with access limited to Project Director and IS Support, and Backup to tape on FAHC server nightly.

2. Web Data Entry Interface

An internet front end is used for entry of lab data that are collected in the individual practices with point of care lab testing devices. These results are not routinely interfaced with the participating lab information systems.

Contents

Result Entry: Add lab results directly into the VDIS database
Order Inquiry: Query or update existing labs previously entered from web interface
   Security
Password protected access is limited to VDIS Operations Staff (passwords hashed on account creation)
Access only available within Fletcher Allen Health Care network
   Functionality
   Data Entry of Laboratory Results
User logs in
Lookup function by name or VDIS identifier
Patient result history appears
User select ‘New Order’ function
Pull-down menus allow for entry of lab results (with date of service)
Optional suppression of alerts to patient or provider (this is included so that old lab results do not result in an alert).
   Order Inquiry:
User logs in
Query database for existing labs by various identify criteria (Accession number, order date, patient, test code)
Results matching search criteria are displayed
   Order Inquiry-update
User logs in
Query database for existing labs by various identify criteria (Accession number, order date, patient, test code)
Results matching search criteria are displayed
User select Order Number to update
Order details are displayed
User makes update and saves changes to the database
   D. Routine Operations and Reports
   Laboratory Enrollment and Start Up
   Sign the VDIS Participation Agreement, Typically at the CEO Level.
   Identify Lab and Technical Contacts for this Project.

Infrastructure will determine the best connection

Lab contact will own connection setup task

Work with FAHC IS Project Management to Review Data Collection Template.

Determine Technical Connection Details.

Current options include: T1, VPN, FTP, HTTPS, GPG/PGP, Secure web service client, etc.

Test the Secure Connection.

Submit Provider Listing Per Template (contacts.xls).

This can be done in parallel with above steps.

Provider Recruitment

The Principal Investigator or Project Director will speak with the primary care providers and recruit them for the study. At this time practices will sign a practice agreement.

Generate and Submit Initial Patient List

When a practice signs on, in order to determine what patients have diabetes, we need the following information for any patient who has had an AlC done in the last 2 years: First Name, Middle Initial, Last Name, MRN, Date of Birth (formatted mm/dd/yyyy), Gender, Marital Status, Address Line 1, Address Line 2, City, State, Zip, Patient Phone Number, Provider (Physician), AlC result, and AlC date of service (formatted mm/dd/yyyy). An example of this information is shown:
M88888888,PUBLIC,JANE,Q,01/20/1944,F,07/16/2004,0716:U00024R,
MICROALBUMIN,MICROALBUMIN,,,,1010, mg/gm,,<30,07/16/2004,07/16/2004
M88888888,PUBLIC,JANE,Q,01/20/1944,F,08/06/2004,0806:C00109R,
CREA,CREA,,,,1010,mg/dL,1,1,08/06/2004,08/06/2004
M88888888,PUBLIC,JANE,Q,01/20/1944,F,08/06/2004,0806:A00014R,
AlC,AlC,,,,1010,%,9,9,08/06/2004,08/06/2004
M99999999,PUBLIC,JOHN,Q,03/19/1956,M,12/07/2002,1207:C00047U,
CREA,CREA,,,,1010,mg/dL,1.8,1.8,12/07/2002,12/07/2002
M99999999,PUBLIC,JOHN,Q,03/19/1956,M,12/07/2002,1207:C00041S,
CREA,CREA,,,,1010,mg/dL,2.7,2.7,12/07/2002,12/07/2002
M99999999,PUBLIC,JOHN,Q,03/19/1956,M,12/08/2002,1208:C00020U,
CREA,CREA,,,,1010,mg/dL,1.2,1.2,12/08/2002,12/08/2002.
   Initial Patient List Review

Initial patient list is formatted for PCP review to identify that patients have diabetes and that they are members of the practice, and that they are not cognitively impaired (eligible to participate). For example, AlC Test Results for patients of PETER PROVIDER, MD:

First Name,Middle Name,Last Name,Birth Date,Sex,Martial Stat,Address 1,City,State,Zip,Phone,NUM,Med Rec Num,DATE,TEXT,

JOHN,Q,PUBLIC,11/15/1945,M,S,12 ELM ST.,BURLINGTON,VT,05450,(802) 555-1212,300117,129985,09/16/2002,HgbAlc,7.7 H

JANE,Q,PUBLIC,12/15/1934,F,M,12 OAK ST,WINOOSKI,VT,05492,(802) 555-1212,300117,53721,09/12/2003,HgbAlc,5

JANE,Q,PUBLIC,12/15/1934,F,M,12 OAK ST,WINOOSKI,VT,05492,(802) 555-1212,300117,53721,10/11/2002,HgbAlc,5.4

JANE,Q,PUBLIC,12/15/1934,F,M,12 OAK ST,WINOOSKI,VT,05492,(802) 555-1212,300117,53721,01/17/2003,HgbAlc,5.7

BOB,F,PUBLIC,07/25/1941,F,M,12 BIRCH ST,MILTON,VT,05465,(802) 555-1212,300117,133609,02/28/2003,HgbAlc,7.1 H

Finalized MRN List and Historical Data Load

Once the initial patient list is reviewed with the PCP, an Excel format file containing the MRN and names of these eligible patients is created. Patient demographic information is extracted from the initial Patient list. Only those patients on the reviewed list are loaded into the VDIS database. At this time, VDIS numbers are assigned to a patient.

The finalized MRN list is provided to the lab in order to produce the Initial Historical Data Load, which is a two-year history of each patient (on the file) for the following test results: AlC, Serum Creatinine, Urine Microalbumin to creatinine ratio, Total Microalbumin, Serum Total Cholesterol, LDL Cholesterol, HDL Cholesterol, and Triglycerides.

The result codes and normal, high and low ranges for the above named tests are to be entered. Please note that these results may exist as single tests or within panels. They may also sometimes require other items to calculate them. The VDIS system requires that we collect these results under all of these conditions. For example, they may appear in panels, such as: 80048 Basic Metabolic Panel, 80053 Comprehensive Metabolic Panel, 80061 Lipid Profile, 80050 General Health Profile, 80069 Renal Panel or other locally-defined panels.

Below are the data columns required per lab test to be imported into VDIS: (An example of this file can be seen in Table 4)
Patient Identifier (MRN), Patient Last name, Patient First name, Patient Middle
Initial, Date of Birth, Sex, LIS Specimen Collect date, LIS Accession number, Ordering Provider, Unit, Lab Result value, Associated Text value, LIS Specimen Receive date, and LIS Specimen Result Date, and Subsequent patients after initial load.

During the course of the study it is likely that patients will be added. At that time we would need the historical data on this patient. Then they should be added to the daily upload. This process will depend on the frequency of new patient additions and may occur weekly, monthly or less often.

TABLE 4
Daily Extract Fields

| Data element | Data format requirements | Example data | Required | Description |
|---|---|---|---|---|
| PATIENT_ID | {alphanumeric, maxlength 20} | 12345 | Y | MRN, SSN or other |
| LAST_NAME | {alphanumeric, maxlength 40} | John | Y | |
| FIRST_NAME | {alphanumeric, maxlength 40} | Doe | Y | |
| MIDDLE_NAME | {alphanumeric, maxlength 40} | David | N | |
| DOB | mm/dd/yyyy | 12/02/1942 | Y | |
| SEX | {alphanumeric, maxlength 10} | M | Y | M/F |
| LIS_COLLECT_DATE | mm/dd/yyyy (time optional) | 06/08/2001 8:14 | Y | Date of Service |
| LIS_ACCESSION_NUM | {alphanumeric maxlength 15} | A123 | Y | |
| SERVICE_CODE | {alphanumeric maxlength 15} | HGBA1C | Y | Lab specific Unique Test |
| | | | | Identifier |
| PARENT_CODE | {alphanumeric maxlength 15} | HGBA1C | N | Lab specific Unique Test |
| | | | | Identifier for parent of the |
| | | | | test |
| ORDERING_PROV_ID | | 123 | N | Physician ID - |
| | | | | Numeric ID For Contact who |
| | | | | placed the test order |
| ORDERING_CLIENT | | 123 | N | Ordering Practice - |
| | | | | Numeric ID for Contact that |
| | | | | will responsible for the order. |
| UNIT | {alphanumeric maxlength 10} | mg/dl | Y | Denotes unit of test result |
| TEST_RESULT_DETAIL_NUMERIC | Numeric Test Result Value | 4 | Y | |
| TEXT | {alphanumeric maxlength 255} | | Y | Text Result/Result |
| | | | | comments |
| LIS_RECEIVED_DATE | mm/dd/yyyy (time optional) | 06/08/2001 10:41 | Y | Date specimen accessed in |
| | | | | Lab |
| RESULT_DATE | mm/dd/yyyy (time optional) | 6/8/2001 10:41:54 AM | Y | Date result finalized |

Daily Upload Start

Once the Historical Data load is received the daily upload should begin. Data required is detailed herein. See the Daily Lab data Extract creation section for a detailed discussion on creation of the daily VDIS extract.

Notification of Lab Customer Service

The results are collected and faxes are sent out very early in the AM to the physician offices. An operational goal is to have all reports created on the previous day's data faxed to the practice before the start of operations for the day. Providers will sometimes receive VDIS reports before standard lab reports. Lab customer service staff are made aware of this at the time the system is started to avoid any confusion if inquiries are made prior to lab reports being received by the physicians. VDIS relies on the timely delivery of accurate lab data from participating labs.

Daily Lab Data Extract Creation

The participating lab is responsible for creating an extract process from their LIS to capture the lab results for participating VDIS patients. The extract should only contain information for consenting VDIS patients. This process should be automated completely to avoid any manual procedural steps. The list of consenting patients will be provided by the VDIS project coordinator.

The lab data is to be in a file with consistent format and delivered daily. The file should be in format is an ASCII, CSV file and contain all resulted (finalized) labs from the previous day (12:00 am to 11:59 pm). A consistent naming convention should be used to identify each daily file.

Identification of LIS Data

Specific, one time tasks must be performed after a lab consents to participate in the VDIS study. These tasks prepare the lab for daily flow of consistent data on a timely basis. Lab staff verifies that data values from within the LIS are correctly mapped to the data values expected by VDIS. This is a one time process that should be performed early in the process of configuring the lab within VDIS.

Patient

Patients are identified by a unique identifier. Valid types of identifiers include medical record numbers (MRN), Social Security numbers (SSN) or something else. Patients can have labs performed at multiple participating labs. A patient can be identified in VDIS with a unique identifier per type per lab. For example, John Smith can be identified in VDIS with the following information:

| Lab | Identifier | Type | Patient |
|---|---|---|---|
| Medical Center X | M998877 | MRN | John Smith |
| Hospital 1 | 123-45-6789 | SSN | John Smith |
| Medical Center Y | M334455 | MRN | John Smith |

If a lab internally identifies a patient by multiple MRNs, a single MRN may be determined before that patient is accepted into VDIS. That MRN (or other identifier) should identify the same patient for entire time the patient is in VDIS unless we are notified by the lab otherwise.

If for some reason VDIS can't match the incoming MRN to an MRN in the VDIS database, VDIS attempts to match the incoming lab to a VDIS patient by full match of:
Last name
First name
Date of Birth
Sex

Patients should have a single unique name (first and last name) in VDIS. Throughout the history of the patient's lab data the patient may have different names due to marriage or the way the hospital intake personal may have entered them. Any discrepancy can be resolved by contacting the patient's practice.

Lab Results

Preferably finalized lab results should be included in the extract. A daily extract should contain the results finalized with the previous day (12:00 am to 11:59 pm) regardless of collection date.

Test Code

VDIS reports on the results of these tests: AlC, Serum Creatinine, Urine Microalbumin to creatinine ratio, Total Microalbumin, Serum Total Cholesterol, LDL Cholesterol, HDL Cholesterol, and Triglycerides.

All possible test codes yielding these specific results that are performed by the lab or sent out of the lab for testing at reference labs should be captured and reviewed by the VDIS project coordinator. The VDIS project coordinator will determine if the test is relevant (should be configured into VDIS).

LIS Accession Number

A unique identifier of the specimen assigned at collection time is used to track results in VDIS.

Test Results

VDIS captures numeric lab results for analysis. However, some results have an alphanumeric representation. This data is captured also.

Numeric Representation

An interpretable numeric exists for each lab result except where the result is an alphanumeric value.
   Alpha Numeric Representation
A set of allowable alphanumeric lab values for reportable tests is determined at before go-live. These are alphanumeric values that are acceptable to import into VDIS.
Values prefaced with a ‘<’ or ‘>’ represent results that are outside the analytic range. These values are captured.
When a Total Microalbumin is outside the analytic range, no Urine Microalbumin to creatinine ratio is calculable. In this situation, there should be a corresponding ratio record that has predefined message stating this. The ratio record is included in the extract to state that the test was performed. The predefined message should be included in the alphanumeric exception list.
When a Triglyceride test is over 400, an LDL is incalculable. However, the LIS will produce a corresponding LDL record stating this, if possible. This lab record helps acknowledge that the test was performed. The predefined message should be included in the alphanumeric exception list.
   Dates

Three dates are used for each lab in the extract:
Date of service (specimen collect date)
Date specimen is received into the lab
Date lab is finalized (resulted)
A time component is not required but is recommended. The date format used is consistent in every extract once it is initially established.
   Format of daily extract

The order of data columns and the column delineators is generally consistent in every extract. If no data exist for the participating patients for the extract period the lab may send a blank file.

Reference Lab Data

Any results from Reference labs are captured. If the participating lab and the reference labs are not interfaced, there may be a lag from the date the lab is finalized at the reference lab to when the information is received at the participating lab. This data is included in a daily extract file.

Transfer Methods

VDIS receives or retrieves the participating laboratory's extract data through various methods. The predominant method is to use FTP to transfer files over a secure network. Other methods include a web service client, scripts to simulate HTTP sessions, manual download via an HTTP session and use of GPG encryption for secure emails.

FTP Over Secure Network

A VPN is configured between FAHC and the participating lab. FAHC network administrators work with the network administrators of the participating lab. An FTP client at the lab connects to the IP of the VDIS FTP server over the VPN. The extract file is then transferred via FTP over the VPN.

The IS contact at the participating lab may request access to the VDIS FTP server from IS security. FAHC Unix administrators will create the account and root directory on the VDIS FTP server.

VDIS Web Service Client

The performing lab exposes a port for their web service on the internet. They supply a WSDL document that defines communicate with their web service. We create a client program that can retrieve the file or data from their site. The client uses HTTPS for secure session communication.

The Extract File is Saved to the VDIS FTP Server.

Script Retrieving Data from Website

The performing lab posts a file on a secure website. The file can be manually downloaded or a script may be created to automate the process. The script uses HTTPS for secure session communication. The extract file is saved to the VDIS FTP server.

Zix Messaging

One of our participating labs uses Zix messaging for secure email communication. They send an email with VDIS as the recipient. Zix intercepts the outgoing email and moves it to the Zix message center. We are notified upon its availability at the Zix message center. We can either manually download or use a script to automatically retrieve the data file. The script uses HTTPS for secure session communication. The extract file is saved to the VDIS FTP server.

If Zix or a similar product is used, the lab may use the vdis-data@pathline5.fahc.org email address.

GPG Secured email (ssmtp)

GPG is the open source (freely available) implementation of PGP encryption. GPG encrypts data in the email with a public key before it is sent. When it is received, it is decrypted with our private key. We release our public key to participating labs (1024 bit key DSA encryption). The extract file is saved to the VDIS FTP server. The email should be addressed to vdis-data@pathline5.fahc.org.

Post FTP Processing

A process on the VDIS FTP Server polls the respective lab's root directories for an extract file every minute. If files are found, the following process takes place:
1) If a file of the same name has been processed before, it will be moved to the lab's exception\directory and an email notification will be sent to VDIS Support.
2) Each record of the extract file is verified that it is not an exact duplicate of a lab result previously processed. If it has been processed already, it is removed from the extract file.
3) Lab specific character replacements or removal with in the extract file are done.
4) The file is Ftp'd to the VDIS data import server.

FIG. 5 outlines the Data site file processing.

Practice Enrollment

IT Component

Performing lab is configured first within the VDIS database before labs can be imported.
Practice information is added to the VDIS database. This consists of Practice name, Practice address, Phone number, Fax number, and Refractory Period.
Provider information is added. This consists of: Provider first name, last name and middle initial, Provider title, Practice affiliation, and Phone, Fax and Cell numbers.
   Associate the Contact with Practice
Load new patient demographic data. Set Loaded patient's status to pending.
Obtain and import patient historical data from the performing lab.
Suppress Flow-sheet and Alert printing on the historical data of these new patients.
   Any labs loaded up to the go-live date must have flow-sheet and alert reporting suppressed. This is done by setting the last_report_sent records as ‘sent’ for these labs.
   Disable the fax process. Run the provider and patient reminder script to start the process that will assign the reminder_sent dates. This will start the cycle of reminders for each patient that are currently overdue for a test. Ensure there are no pending report_log records. Enable the fax process.
   Add new patients to daily lab extract.
      Send Finalized MRN List Only to the Lab in Contact.
   Determine when the patients will be added to the feed. This is the go-live date.
   These scripts and instructions are in the supporting documentation/New Practice Enrollment.doc document.
      Operations Component
   The Operations component includes a signed agreement, signed consent form, review of patient rosters, consent letter mailed by VDIS staff, and if there is no response in 10 days, the roster is finalized, followed by a randomization step.
   Master Randomization Envelopes are stored in the VDIS secure file cabinet.
   Practice is assigned to a numbered envelope in the order they are randomized.
   Practice name is written on the outside of the envelope.
   Envelope is opened and practice name is written on the numbered card which lists assignment to intervention or control.
   Envelope and card are stored in the VDIS secure file cabinet.
   Providers are notified by letter prior to practice start—see Notification letters (intervention and control)
   See Practice Changes, System Start Up for details of start up process
      Reports
      IT Component

The Flow-sheet and Patient Alert creation process is run every 15 minutes throughout the day to promptly report on recently loaded data. This interval is configurable.

The Patient Reminder and Provider Reminder creation process is run once early in the morning. This time is configurable.

Flow Sheet

A flow-sheet is created for every patient that has had a recent Microalbumin, Creatinine, AlC or Lipid panel lab result imported into VDIS. Specifically, for every patient that has:

an Active status;

a recently imported lab with a test code of UAB, CRE, TRIG, AlC or LDL that:
has not been reported yet
has a value (is not blank)
   Process

Get each patient's history for the reportable tests. Specifically, get each test in the patient's history that:
has never been reported on a flow-sheet before,
has a lab value (is not blank)

Create the report. Report only the last four labs of each test code.

Review the result range and the overdue status of the result. Get the recommendation text for each test depending upon the result_range and overdue status of the result.

Determine LIPID recommendation text by examining the result dates of the component LDL and TRIG results.

Flow sheets are faxed to the provider. They are faxed in batch, usually within 15 minutes of being created.

Provider Alert

A Provider Alert is created for every patient that:
has an active status and

is ‘overdue’ for one or many labs. A patient is over due if the date the last reminder was sent in the past, is older than the refractory_period. Specifically:

SYSDATE >
(NVL(PHYSICIAN_REMINDER_SENT,TO_DATE(‘1/1/1900’,‘MM/DD/YYY
Y’)) + PATIENT_REFRACTORY_PERIOD)
(The patient_refractory_period is practice specific.)

The latest Microalbumin, Creatinine, AlC, or Lipid result is reviewed. If it is older than the grace_period+the overdue period (defined in the diabetes_test_overdue_periods table and determined by test code and result range) or if the specific test is missing, a reminder is created.

Provider Alerts are faxed to the provider. They are faxed in batch, usually within 15 minutes of being created.

Patient Reminder

A Patient Reminder is created for every patient that:
has an active status and

is ‘overdue’ for one or many labs. A patient is over due if the date the last reminder was sent in the past, is older than the refractory_period. Specifically:

SYSDATE >
(NVL(PATIENT_REMINDER_SENT,TO_DATE(‘1/1/1900’,‘MM/DD/YYYY’))
+ PATIENT_REFRACTORY_PERIOD)
(The patient_refractory_period is practice specific.)

The latest Microalbumin, Creatinine, AlC, or Lipid result is reviewed. If it is older than the grace_period+the overdue period (defined in the diabetes_test_overdue_periods table and determined by test code and result range) or if the specific test is missing, a reminder is created.

Patient Reminders are mailed to the patient the day they are created (see details of mail and production).

Patient Alert

A patient alert is sent if a Microalbumin, AlC or LDL is out of control and:
if the last Microalbumin was high or there was 2 medium Microalbumins in not necessarily in a row and The patient was never alerted for Microalbumins before. Only one Microalbumin alert is sent to the patients.
or the AlC is high
or the LDL is high if and only if it is not high due to a high TRIG.

Patient Alerts are created within 15 minutes of receiving the data. They are mailed the following day.

Population Report

User signs into the web interface with administrator account

User selects a single practice

User selects one or many providers

The population report application creates the report then displays the report in a browser window. A copy is saved to under the ouputFiles/reports/population under the VDIS application root on the production server.

Three tests are reported on (AlC,UAB and LDL). For each test:

Calculate the entire sample Achievable Benchmark for Care (ABC) for the high, medium and low ranges, the ontime sample and the Vermont (and NY) sample

Get the appropriate high, medium, low result range and ‘ontime’ labels for the report.

Get the Vermont (and NY) high, medium and low sample percentage.

Get the Vermont (and NY) on time sample percentage.

// end for each test

For each provider selected,

Get the provider name and sample (patients).

Then for each test:

Calculate the high, medium, low result range and ontime totals the for physicians sample

Then for each patient:

get the lab value and it's associated result range

// end each patient

// end each test

// end each provider selected.

The ABC calculation procedure is on file.

The Percent ontime is calculated by dividing the number of patients in a sample who are not overdue by the total Vermont (and NY) sample.

Operations Component

Production of Population Reports by Practice and by PCP
Population reminder is generated from the Practice Database every 3-4 months.
Secty runs tickler system weekly
Secty logs on to VDIS and produces population report, by physician.
Secty mails reports providers, each in a separate envelope for confidentiality, with cover letter “VDIS Population Report Instructions”
   Monitoring
   IT Based Monitoring—VDIS Monitor
Gathers metrics about the daily import stream of labs on the day that it is run.
Currently scheduled to run 9:05 am
Java application in WLS1:/opt/bea/Apps/NVDIS/vdismonitor
Output is emailed to vdissupport@fahc.org, with exception attached in a csv file.
Output is written to 3 csv files—labs.csv, reports.csv and exceptions.csv. They are date stamped and are moved to the VDIS Control Staging directory. These data can then be imported into Ben's control monitoring spreadsheet.
Control limits are calculated from Ben spreadsheets. The limit values are also stored in the VDIS oracle database. This allows the VDISMonitor support email to notify us of out of control measures
The email lists errors first and marks the email as urgent if there are errors. Any errors require attention. Currently the errors are:
   1. No file exist from a lab.
   2. A file exists but 0 records appear in the second metric. (possible duplicate from lab.)
   3. A file exists but 0 records are imported into the db.
   4. A metric is out of the control limits.
      Other Systematic Checks
CDM practice will function as a test of system.
For every lab test which generates a standard lab report, a fax is generated from VDIS.
Periodic checking of the daily output.
IFSCO folder checked every weekday for duplicates, prior to printing.
VDIS IS Support will To Whom It May Concern: check fax output daily, until bugs are resolved.
   Error Log

The error log resides within the database. Every VDIS process writes to it whenever an error, fatal or non-fatal occurs. This generates an email to IS Support, which is an active stimulus to investigate the error.

H. System Software

Requirements

Web Front end

AIX v2.5
Weblogic J2EE Application server v7.4
Oracle v9.0.1
JDK v.1.3.1
Itext v0.90
log4j v1.2.8
   Report Creation Module
AIX v2.5
JDK 1.3.1
GNU hylafax v0.0.7
Hylafax v.4.2.1
Itext v.0.90
log4j v1.2.8
   VDISMonitor
JDK 1.3.1 (for AIX v2.5)
Oracle v9.0.1
   Web Service client
JDK1.3.1 (forAIXv2.5)
Sun's WebServices development package v1.3 (jwsdp-1_{—}3-windows-i586.exe)
   Monitoring Scripts
   Change Tracking

All software enhancement requests and bug fixes are tracked within our local installation of iTracker. This allows us to identify and store all attributes of the enhancement or bug fix and track the history of associated system changes.

Software Version Control

All software versioning is maintained by Merant's PVCS change control software. Software is checked out of PVCS into a developer's ‘sandbox’ (local development environment). Once the change is made and properly QA'd, each source file is then checked back into PVCS with appropriate comments and an associated iTracker issue number. This allows quick root cause analysis if any regression takes place.

Hypertension Protocol

Rationale

If a patient has a blood pressure indicative of hypertensive emergency appropriate action should be taken.

Definitions & Notes

Diagnosis of HTN emergency calls for a history to be obtained which is beyond the scope of the RA, so a telephone consultation with the supervising clinician is used. There is no consensus on a single threshold defining a hypertensive emergency. Severe HTN is defined as diastolic BP>130, which we do not anticipate seeing ever.

In order to keep protocol simple we will trigger off any BP above threshold and not require computation of an average BP by the RA.

We will pick a threshold that is lower than the definition of severe hypertension and at least have a conversation with the patient to determine the urgency of follow up.

Trigger: If BP>220 Systolic or >110 Distolic

Action by RA: Call supervising MD.
   From UpToDate:

Severe asymptomatic hypertension (hypertensive urgencies)

UpToDate performs a continuous review of over 300 journals and other resources. Updates are added as important new information is published.

INTRODUCTION—Severe hypertension (as defined by a diastolic blood pressure above 130 mmHg) can produce a variety of acute, life-threatening complications. These include hypertensive encephalopathy, malignant nephrosclerosis, retinal hemorrhages, and papilledema. (See “Hypertensive emergencies: Malignant hypertension and hypertensive encephalopathy” and see “Treatment of specific hypertensive emergencies”).

Some patients, however, are asymptomatic despite an equivalent degree of hypertension. This entity has been called a “hypertensive urgency” and a relatively rapid reduction in blood pressure (BP) has in the past been recommended.

A variety of oral therapeutic modalities have been used in this setting, including an hourly clonidine loading regimen (0.1 to 0.2 mg followed by 0.05 to 0.1 mg every 1 to 2 hours to a maximum dose of 0.7 mg), sublingual nifedipine (2.5 to 10 mg), and oral or sublingual captopril (6.25 to 25 mg).

There is, however, no proven benefit from rapid reduction in BP in asymptomatic patients who have no evidence of acute end-organ damage and are at little short-term risk. Furthermore, cerebral or myocardial ischemia or infarction can be induced by aggressive antihypertensive therapy if the BP falls below the range at which tissue perfusion can be maintained by autoregulation. This is most likely to occur with sublingual nifedipine capsules; the degree of blood pressure reduction cannot be controlled or predicted with this preparation and severe ischemic complications have rarely been reported.

RECOMMENDATION—The initial goal in patients with severe asymptomatic hypertension should be a reduction in blood pressure to 160/110 over several hours with conventional oral therapy. The simple combination of rest in a quiet room and, if the patient is not volume depleted, a loop diuretic can lead to a fall in BP to a safe level in many patients. With furosemide, for example, the dose is 20 mg if renal function is normal, and higher if renal insufficiency is present. This can be given with an oral calcium channel blocker (isradipine, 5 mg or felodipine, 5 mg) since almost all such patients require therapy with at least two antihypertensive medications. A dose of captopril (12.5 mg) can be added if the response is not adequate.

This regimen should lower the blood pressure to a safe level over three to six hours. The patient can then be discharged on a regimen of once-a-day medications, with a close follow-up to ensure adequate treatment.

Most patients with relatively severe hypertension (diastolic pressure≧20 mmHg), have no acute, end-organ injury. Although some propose relatively rapid antihypertensive therapy in this setting (as with sublingual nifedipine or oral clonidine loading), there may be more risk than benefit from such an aggressive regimen.

Malignant Hypertension

INTRODUCTION—Hypertensive emergencies are acute, life-threatening, and usually associated with marked increases in blood pressure (BP). There are two major clinical syndromes induced by the severe hypertension:
Malignant hypertension is marked hypertension with retinal hemorrhages, exudates, or papilledema. There may also be renal involvement, called malignant nephrosclerosis. Although papilledema had been thought to represent a more severe lesion, it does not appear to connote a worse prognosis than hemorrhages and exudates alone (so-called accelerated hypertension). Thus, treatment is the same whether or not papilledema is present.
Hypertensive encephalopathy refers to the presence of signs of cerebral edema caused by breakthrough hyperperfusion from severe and sudden rises in blood pressure.

CLINICAL MANIFESTATIONS—Malignant hypertension most often occurs in patients with long-standing uncontrolled hypertension, many of whom have discontinued antihypertensive therapy. Underlying renal artery stenosis is also commonly present, particularly in white patients.

In addition to marked elevation in BP, the major clinical manifestations include.

Retinal hemorrhages and exudates (representing both ischemic damage and leakage of blood and plasma from affected vessels) and papilledema.
Malignant nephrosclerosis, leading to acute renal failure, hematuria, and proteinuria. Renal biopsy reveals fibrinoid necrosis in the arterioles and capillaries, producing histologic changes that are indistinguishable from any of the forms of the hemolytic-uremic syndrome. The renal vascular disease in this setting leads to glomerular ischemia and activation of the renin-angiotensin system, possibly resulting in exacerbation of the hypertension.
Neurologic symptoms due to intracerebral or subarachnoid bleeding, lacunar infarcts, or hypertensive encephalopathy. The last problem, which is related to cerebral edema, is characterized by the insidious onset of headache, nausea, and vomiting, followed by nonlocalizing neurologic symptoms such as restlessness, confusion, and, if the hypertension is not treated, seizures and coma. Magnetic resonance imaging (particularly with T2-weighted images) may reveal edema of the white matter of the parieto-occipital regions, a finding termed posterior leukoencephalopathy.

### VDIS Database Understanding

This example aims at capturing the list of tables that need to be populated as a part of the VDIS project. The document outlines the relationships that exist between tables in the existing OCMS schema.

The document is divided into sections, which indicate the entities that need to be populated in the database from the VDIS perspective. Comments and Queries have been provided where necessary to highlight issues that need to be resolved.

The information is represented in a tabular format, which is explained below:

TABLE
Table Name

| Column | | | Data | FK | FK | | | |
|---|---|---|---|---|---|---|---|---|
| Name | Constraint | Null? | Type | Table | Column | Description | Needed? | Comments |
| Name of | Name of | Nullable? | Data | Referenced | Referenced | Purpose of | Indicates if | Comments |
| the | Referential | Yes/No | Type for | Table | Column in | the column | the column | regarding |
| column | Constraint | | column | | Referenced | | needed for | the |
| | if any | | | | Table | | populating | population |
| | | | | | | | VDIS data | of data in |
| | | | | | | | | the column |
| | | | | | | | | from VDIS |
| | | | | | | | | perspective |

Summary of Database Tables
Population of Test Results

Table: TEST_RESULT

Post Discussion Changes/Clarifications

Table: TEST_ORDE

Table: TEST RESULT DETAIL

Mandatory Master Tables

Population of Service Tables

Table: SERVICE_DIRECTORY

Table: SERVICE_PROFILE

Table: SERVICE_PROVIDER

Table: SERVICE_ORDER

Mandatory Master Tables

Population of Patient Tables

Table: PATIENT

Table: ALTERNATIVE_PATIENT_ID

Table: PATIENT_EVENT

Mandatory Master Tables

Population of Miscellaneous Tables

Table: CONTACT

Table: ALTERNATE_CONTACT_ID

Table: CLIENT

Table: WORKS_FOR_EMPLOYS

Table: ORGANIZATION

Table: SENDING_APP

Table: RELATED_TO

Table: LOCATION.

VDIS Specific Tables

Master Tables

Table: PATIENT_STATUS LOOKUP

Table: REPORT_LOOKUP

Table: PARSER_CLASS_LOOKUP

Table: FILE_STATUS_LOOKUP

Table: MODULE_LOOKUP.

Table: OPERATION_TYPE_LOOKUP

Table: REPORT_STATUS_LOOKUP

Table: OUTPUT_TYPE_LOOKUP

Table: CANNED_TEXT_LOOKUP

Detail Tables

Table: REPORT_LOG

Table: PATIENT_STATUS_CHANGE_HISTORY

Table: FILE_DATA

Table: FTP_CONFIG

Table: LIS_DATA

Table: GRACE_PERIOD.

Table: DIABETES TEST_REF_RANGE

Table: CLIENT_DATA

Table: LAST_REPORT_SENT

Table: TEST_GENERATION_LOG

Table: FS LIPID_TRUTH_TABLE

Table: FS_AlC_TRUTH_TABLE

Table: FS_MC_TRUTH_TABLE

Table: REMINDER_TRUTH_TABLE

Table: DIABETES_TEST_OVERDUE_PERIODS

Remaining tables

Population of Test Results

TABLE TEST_RESULT
During the order entry process, record is entered for every resultable test.

| Column Name | Constraint | Null? | Data Type | FK Table | FK Column | Description | Needed? | Comments |
|---|---|---|---|---|---|---|---|---|
| SERVICE_ORDER_ID | ORDER_HAS_RESULT | No | INTEGER | SERVICE_ORDER | SERVICE_ORDER_ID | Order Id, | Yes | Order Id, |
| | | | | | | Internal to | | Need to |
| | | | | | | VDIS | | internally |
| | | | | | | | | generate for |
| | | | | | | | | VDIS. LAB |
| | | | | | | | | OrderID will |
| | | | | | | | | be sent along |
| | | | | | | | | with Results |
| | | | | | | | | from FTP |
| | | | | | | | | upload |
| | | | | | | | | hospitals, |
| | | | | | | | | HL7, |
| | | | | | | | | Webforms. |
| | | | | | | | | One unique |
| | | | | | | | | order_id |
| | | | | | | | | would be |
| | | | | | | | | generated per |
| | | | | | | | | Accession |
| | | | | | | | | Number |
| RESULT_ID | | No | INTEGER | | | Running | Yes | Need to |
| | | | | | | sequence id | | internally |
| | | | | | | for results for | | generated for |
| | | | | | | a order | | VDIS. One ID |
| | | | | | | | | per |
| | | | | | | | | Resultable |
| SERVICE_PROVIDER | HAS_RESULT | No | INTEGER | SERVICE_DIRECTORY | SERVICE_PROVIDER_ID | Service | Yes | NECLA Labs. |
| | | | | | | Provider Id | | Say Rutland, |
| | | | | | | | | FAHC |
| SERVICE_CODE | | No | VARCHAR(15) | | SERVICE_CODE | Service Code | Yes | Refer to |
| | | | | | | | | SERVICE_DIRECTORY. |
| | | | | | | | | It |
| | | | | | | | | has master |
| | | | | | | | | info for all |
| | | | | | | | | tests. |
| PARENT_CODE | | No | VARCHAR(15) | | | Parent Test | Yes | Refer to |
| | | | | | | Code for the | | SERVICE_DIRECTORY & |
| | | | | | | service code | | Service |
| | | | | | | | | Profile. It has |
| | | | | | | | | master info |
| | | | | | | | | for all tests. |
| ORDERING_PROV_ID | ORDER_PROV_FOR | | INTEGER | CONTACT | CONTACT_ID | Ordering Site | No | This column |
| | | | | | | | | will need to |
| | | | | | | | | store the |
| | | | | | | | | Ordering |
| | | | | | | | | Provider/ |
| | | | | | | | | LAB ID |
| PATIENT_ID | HAS | No | INTEGER | PATIENT | PATIENT_ID | Patient | Yes | Internal VDIS |
| | | | | | | Identifier | | identifier |
| RESULT_DATE | | | TIMESTAMP | | | Result Date | Yes | This would be |
| | | | | | | | | defaulted to |
| | | | | | | | | the day the |
| | | | | | | | | results were |
| | | | | | | | | parsed by |
| | | | | | | | | VDIS |
| REFERENCE_RANGE | | | VARCHAR(25) | | | Range for the | Yes | Predefined |
| | | | | | | test result | | normal |
| | | | | | | values | | reference |
| | | | | | | | | range |
| UNIT | | | VARCHAR(10) | | | Units of the | Yes | Unit of |
| | | | | | | range | | measurement. |
| | | | | | | | | e.g. For LDL |
| | | | | | | | | it is mg/dl |
| INTERP_CODE | | | VARCHAR(10) | | | Interpretation | Yes | Interpretation |
| | | | | | | of code L, H, | | of code Low/ |
| | | | | | | R, S, CL, CH . . . | | High/ |
| | | | | | | | | Medium, etc. |
| | | | | | | | | Cross - |
| | | | | | | | | referencing |
| | | | | | | | | may be |
| | | | | | | | | required? |
| NOTE | | | VARCHAR(10) | | | Not Used | No | |
| REPORTABLE_NOTE | | | VARCHAR(10) | | | Not Used | No | |
| CANCEL_REASON | | | VARCHAR(255) | | | Cancellation | No | |
| | | | | | | Reason | | |
| STATUS | STATUS_OF | | VARCHAR(10) | RESULT_LOOKUP | CODE | Final Cancelled | Yes | VDIS will |
| | | | | | | or incomplete | | accept the |
| | | | | | | | | results only |
| | | | | | | | | with FINAL |
| | | | | | | | | status |
| REPORT_STATUS | REPORT_STATUS_OF | | VARCHAR(10) | RESULT_LOOKUP | CODE | Not Used | No | |
| ORIGIN | | | VARCHAR(1) | | | Source for | Yes | The values |
| | | | | | | Result. ‘I’ for | | entered |
| | | | | | | Interface. | | would be as |
| | | | | | | | | follows: |
| | | | | | | | | ‘F’ - FTP, ‘I’- |
| | | | | | | | | HL7 |
| | | | | | | | | Interface, |
| | | | | | | | | ‘W’- Web |

TABLE TEST_ORDER
This table contains a list of tests for a particular order.

| Column Name | Constraint | Null? | Data Type | FK Table | FK Column | Description | Needed? | Comments |
|---|---|---|---|---|---|---|---|---|
| SERVICE_ORDER_ID | CONTAINS | No | INTEGER | SERVICE_ORDER | SERVICE_ORDER_ID | | Yes | Order Id, Need to |
| | | | | | | | | internally generate |
| | | | | | | | | for VDIS. LAB |
| | | | | | | | | OrderID will be |
| | | | | | | | | sent along with |
| | | | | | | | | Results from FTP |
| | | | | | | | | upload hospitals, |
| | | | | | | | | HL7, |
| | | | | | | | | Webforms. One |
| | | | | | | | | order ID per |
| | | | | | | | | Accession Number |
| SERVICE_PROVIDER_ID | PROCEDURE_ORDER | No | INTEGER | SERVICE_DIRECTORY | SERVICE_PROVIDER_ID | | Yes | NECLA Labs. Say |
| | | | | | | | | Rutland, FAHC |
| SERVICE_CODE | | No | VARCHAR(15) | | SERVICE_CODE | | Yes | Refer to |
| | | | | | | | | SERVICE_DIRECTORY. |
| | | | | | | | | It has master |
| | | | | | | | | info for all tests. |
| SERVICE_VERSION | | | INTEGER | | | Not used | No | |
| LIS_ACCESSION_NUM | | | VARCHAR(10) | | | Accession | Yes | Needed to group |
| | | | | | | number sent | | results together. |
| | | | | | | by the | | Accession number |
| | | | | | | performing LIS | | sent by the |
| | | | | | | | | performing LIS |
| PRIORITY | | | VARCHAR(10) | | | Not used, | No | |
| | | | | | | Default STAT | | |
| ICD9_CODE | DX_TEST_FOR | | VARCHAR(10) | ICD9 | ICD9_CODE | NULL | No | |
| DUPLICATE_OK | | | VARCHAR(1) | | | Indicates that | No | |
| | | | | | | its OK to order | | |
| | | | | | | the same test | | |
| | | | | | | (with all specs | | |
| | | | | | | same) in the | | |
| | | | | | | same day. | | |
| DECLINE_REFLEX | | | VARCHAR(1) | | | Reflex test Yes/ | No | |
| | | | | | | No flag | | |
| REPORTABLE_COMM | | | VARCHAR(80) | | | Comments | No | |
| NONREPORTABLE_COMM | | | VARCHAR(80) | | | Comments | No | |
| STATUS | TO_STATUS_OF | | VARCHAR(10) | ORDER_LOOKUP | CODE | Stores the | No | |
| | | | | | | status of the | | |
| | | | | | | particular test. | | |
| | | | | | | Values: | | |
| | | | | | | Pending, To | | |
| | | | | | | Lab, In Lab, | | |
| | | | | | | Received, | | |
| | | | | | | Final | | |
| LIS_COLLECT_DATE | | | TIMESTAMP | | | Date the | Yes | This is a useful |
| | | | | | | specimen | | piece of |
| | | | | | | should be | | information for |
| | | | | | | collected. | | study. When was |
| | | | | | | | | the specimen |
| | | | | | | | | supposed to be |
| | | | | | | | | collected? |
| LIS_RECEIVED_DATE | | | TIMESTAMP | | | Date the | No | |
| | | | | | | specimen was | | |
| | | | | | | received at the | | |
| | | | | | | performing | | |
| | | | | | | site. | | |
| BAR_CODE | | | VARCHAR(9) | | | Encoded form | No | |
| | | | | | | of accession | | |
| | | | | | | number sent | | |
| | | | | | | by performing | | |
| | | | | | | LIS and is | | |
| | | | | | | used for | | |
| | | | | | | generating | | |
| | | | | | | Barcodes. | | |
| PERFORMED_AT | | | | SERVICE_DIRECTORY | SERVICE_PROVIDER_ID | Where was the | Yes | At which Lab Test |
| | | | | | | test performed | | is performed. |

TABLE TEST_RESULT_DETAIL

| Column Name | Constraint | Null? | Data Type | FK Table | FK Column | Description | Needed? | Comments |
|---|---|---|---|---|---|---|---|---|
| SERVICE_ORDER_ID | TEST_DETAIL_FOR | No | INTEGER | TEST_RESULT | SERVICE_ORDER_ID | Order Id | Yes | Order Id, Need |
| | | | | | | | | to internally generate for |
| | | | | | | | | VDIS. LAB OrderID will be |
| | | | | | | | | sent along with Results |
| | | | | | | | | from FTP upload |
| | | | | | | | | hospitals, HL7, Webforms |
| RESULT_ID | | No | INTEGER | | RESULT_ID | Sequence Id | Yes | Running |
| | | | | | | for results | | Sequence Id |
| | | | | | | | | for test |
| | | | | | | | | results. |
| | | | | | | | | Generated |
| | | | | | | | | internally for |
| | | | | | | | | VDIS |
| OBSERVATION_SUBID | | No | INTEGER | | | Sub Result Id | Yes | Each |
| | | | | | | | | resultable may have multiple |
| | | | | | | | | observations [OBX in HL7] |
| SEQ_NUM | | No | INTEGER | | | Sequence | Yes | Each |
| | | | | | | Number | | Observation may have |
| | | | | | | | | multiple sub-results(Called |
| | | | | | | | | Nesting in HL7, these are |
| | | | | | | | | separated using a ‘˜’) |
| RESULT_TYPE | RESULT_TYPE_OF | | VARCHAR(10) | RESULT_LOOKUP | CODE | | Yes | String, |
| | | | | | | | | Numeric, |
| | | | | | | | | Coded Entry |
| NUMERIC | | | REAL | | | Contains data | Yes | The numeric |
| | | | | | | if the result | | value of the |
| | | | | | | type is | | test result e.g. |
| | | | | | | numeric | | LDL |
| | | | | | | | | 30.90 mg/dl. |
| NUM_SIGNIF | | | VARCHAR(15) | | | Not used | Yes | Number of significant |
| | | | | | | | | digits in test result value. |
| | | | | | | | | e.g. LDL 30.90 mg/dl. |
| | | | | | | | | The result is |
| | | | | | | | | reported using 2 significant |
| | | | | | | | | digits after the decimal |
| TEXT | | | VARCHAR(254) | | | Contains data | Yes | Test value if |
| | | | | | | if the result | | the result is |
| | | | | | | type is CE | | text. Say in |
| | | | | | | | | case of |
| | | | | | | | | missing |
| | | | | | | | | results. |
| RCOMMENT | | | VARCHAR(254) | | | Contains data | Yes | Recommendations |
| | | | | | | if the result | | in test |
| | | | | | | type is TEXT | | results. |
| REPORT | | | CLOB | | | Contains data | No for CoPATH results | |
| HAS_REPORT | | | VARCHAR(1) | | | ‘Y’ indicates | No value present | |
| | | | | | | in the Report column | | |

Mandatory Master Tables:

| Table Name | Description |
|---|---|
| RESULT_LOOKUP | Lookup for various codes used by result |
| SERVICE_DIRECTORY | Stores information related to Tests |
| CONTACT | Contact related information for organizations |
| PATIENT | Information about patients |

Population of Service Tables

TABLE SERVICE_DIRECTORY
Contains a subset of Procedures. This contains site specific definition of Procedures.

| Column Name | Constraint | Null? | Data Type | FK Table |
|---|---|---|---|---|
| SERVICE_PROVIDER_ID | OFFERS | No | INTEGER | SERVICE_PROVIDER |
| SERVICE_CODE | | No | VARCHAR(15) | |
| SERVICE_DIVISION | SDIV_FOR | | VARCHAR(10) | SERVICE_DIVISION |
| SERVICE_STAT_AVAILABLE | | | VARCHAR(5) | |
| ANALYTICAL_TIME | ANALTIME_FOR | | VARCHAR(10) | SERVICE_LOOKUP |
| SERVICE_LOCAL_NAME | | | VARCHAR(60) | |
| ALPHA_NAME | | | VARCHAR(50) | |
| SHORT_NAME | | | VARCHAR(20) | |
| REFERENCE_TEST_ID | | | VARCHAR(8) | |
| RESTRICTED | | | VARCHAR(1) | |
| ORDERABLE | | | VARCHAR(1) | |
| BILLABLE | | | VARCHAR(1) | |
| PRINTABLE | | | VARCHAR(1) | |
| RESULTABLE | SRESULTABLE | | VARCHAR(10) | SERVICE_LOOKUP |
| INTERFACEABLE | | | VARCHAR(1) | |
| AUTO_UPDATE | | | VARCHAR(1) | |
| MANUAL_RESULTING | | | VARCHAR(1) | |
| DUPSOK | | | VARCHAR(1) | |
| SERVICE_TYPE | STYPE | | VARCHAR(10) | SERVICE_LOOKUP |
| REQUISITION_TYPE | SREQTYPE | | VARCHAR(10) | SERVICE_LOOKUP |
| POLICY_TYPE | POLICY_TYPE_OF | | VARCHAR(10) | COMPLIANCE_LOOKUP |
| TEST_CODE | TEST_OF | | VARCHAR(15) | PROCEDURE |
| METHOD | SERVICE_METHOD_FOR | | VARCHAR(50) | METHOD_LOOKUP |
| TEST_VERSION | | | INTEGER | |
| PROC_VERSION | | | INTEGER | |
| SERVICE_PROFILE_VERSION | | | INTEGER | |
| SERVICE_CODE_VERSION | | No | INTEGER | |
| SERVICE_EFFECTIVE_DATE | | No | DATE | |
| SERVICE_ACTIVE_STATUS | | No | VARCHAR(1) | |
| SPECIAL_HANDLING | | | VARCHAR(1) | |
| COLLECT_NOTE | SERVICE_NOTE_FOR | | VARCHAR(10) | CODED_COLLECT_NOTE |
| PERFORMED_AT | PROVIDES_SRV_FOR | | INTEGER | SERVICE_PROVIDER |
| TEST_COLLECTION_VERSION | | | INTEGER | |

| Column Name | FK Column | Description | Needed? | Comments |
|---|---|---|---|---|
| SERVICE_PROVIDER_ID | SERVICE_PROVIDER_ID | | Yes | NECLA Labs. Say |
| | | | | Rutland, FAHC |
| SERVICE_CODE | | | Yes | It has master infor for |
| | | | | all test codes. |
| SERVICE_DIVISION | DIV_CODE | Type of Lab e.g. | No | |
| | | Chemistry, AP | | |
| SERVICE_STAT_AVAILABLE | | Flag indicates if | No | |
| | | the test can be | | |
| | | performed on the | | |
| | | urgent basis. Not | | |
| | | supported in | | |
| | | Phase I | | |
| ANALYTICAL_TIME | CODE | Time to result | No | |
| SERVICE_LOCAL_NAME | | Descriptive Text | Yes | Can be used for |
| | | | | Reporting. Local lab |
| | | | | name for tests. Say |
| | | | | Rutland calls HgbA1c |
| | | | | for Hemoglobin. |
| ALPHA_NAME | | Name after | No | |
| | | trimming the | | |
| | | leading Numbers | | |
| | | in the | | |
| | | Service_Local_name | | |
| SHORT_NAME | | | Yes | Can be used for |
| | | | | Reporting. |
| REFERENCE_TEST_ID | | Not used | No | |
| RESTRICTED | | This indicates if | No | |
| | | only Provider who | | |
| | | has signed special | | |
| | | authorization can | | |
| | | order the test. | | |
| | | Not Supported in | | |
| | | Phase I | | |
| ORDERABLE | | Orderable Y or N. | Yes | For maintaining test |
| | | Only the | | hierarchies.Would help |
| | | orderable test can | | to indicate what |
| | | used during Order | | resultables are under |
| | | Entry | | an orderable. |
| | | | | Whether test is |
| | | | | orderable. |
| BILLABLE | | Not Used | No | |
| PRINTABLE | | Can it be printed | No | |
| | | as a part of the | | |
| RESULTABLE | CODE | COND/DRUG/Y | Yes | For maintaining test |
| | | | | hierarchies. What |
| | | | | resultables are under |
| | | | | an orderable, whether |
| | | | | the orderable is itself |
| | | | | a resultable or not ? |
| | | | | Whether test is |
| | | | | resultable. |
| INTERFACEABLE | | Y or N. If ‘N’ then | No | |
| | | the requisition | | |
| | | needs to be sent | | |
| | | with the specimen | | |
| AUTO_UPDATE | | Not Used. Update | No | |
| | | automatically | | |
| | | from a interface. | | |
| MANUAL_RESULTING | | Can result be | No | |
| | | entered using | | |
| | | PathLINE. | | |
| DUPSOK | | Check if the | No | |
| | | Duplicates are | | |
| | | okay. Yes or No | | |
| | | has to be checked | | |
| | | both for test | | |
| | | every order and | | |
| | | for every day. | | |
| SERVICE_TYPE | CODE | Test/Battery/ | Yes | Single test/Battery/ |
| | | Package/Reflex/ | | package. |
| | | BillPkg | | Examples, |
| | | | | LDL single test |
| | | | | Lipid battery of LDL, |
| | | | | HDL, TC and Trig tests |
| | | | | Package. Package |
| | | | | may consist of a test |
| | | | | and a battery, |
| | | | | however batteries are |
| | | | | made up only of tests |
| REQUISITION_TYPE | CODE | CYTO/GENLAB/ | No | |
| | | COPATH. Various | | |
| | | types of | | |
| | | requisition. | | |
| POLICY_TYPE | CODE | Medicare Part A or | No | |
| | | Medicare Part B | | |
| TEST_CODE | PROC_CODE | Master Code for | Yes | FAHC specific master |
| | | the service | | test code |
| METHOD | METHOD | Tells method of | Yes | Tells method/process |
| | | test execution. | | of text execution. Say, |
| | | Say, Chemical | | Chemical process |
| | | process | | |
| TEST_VERSION | | Not used | No | |
| PROC_VERSION | | Not used | No | |
| SERVICE_PROFILE_VERSION | | Not Used | No | |
| SERVICE_CODE_VERSION | | Not Used | No | |
| SERVICE_EFFECTIVE_DATE | | | No | |
| SERVICE_ACTIVE_STATUS | | | No | |
| SPECIAL_HANDLING | | ‘Y’ indicates that | No | |
| | | the source/ | | |
| | | specimen needs | | |
| | | special handing at | | |
| | | collection time. | | |
| COLLECT_NOTE | COLLECT_NOTE | COLL_NOTE/ | No | |
| | | TEST_NOTE. | | |
| | | TEST_NOTE not | | |
| | | supported in | | |
| | | Phase I. | | |
| PERFORMED_AT | SERVICE_PROVIDER_ID | Performing site | Yes | ID of performing LAB |
| | | for the Ordering | | |
| | | Provider | | |
| TEST_COLLECTION_VERSION | | | No | |

TABLE SERVICE_PROFILE
This table depicts parent child relationship between tests in SERVICE_DIRECTORY table

| Column Name | Constraint | Null? | Data Type | FK Table |
|---|---|---|---|---|
| PARENT_SERVICE_PROVIDER_ID | SERVICE_PARENT_OF | No | INTEGER | SERVICE_DIRECTORY |
| PARENT_SERVICE_CODE | | No | VARCHAR(15) | |
| CHILD_SERVICE_PROVIDER_ID | SERVICE_CHILD_OF | No | INTEGER | SERVICE_DIRECTORY |
| CHILD_SERVICE_CODE | | No | VARCHAR(15) | |
| CPT_MULTIPLIER | | | INTEGER | |
| SERVICE_PROFILE_VERSION | | No | INTEGER | |
| SORT_ORDER | | | INTEGER | |

| Column Name | FK Column | Description | Needed? | Comments |
|---|---|---|---|---|
| PARENT_SERVICE_PROVIDER_ID | SERVICE_PROVIDER_ID | | Yes | Parent Provider ID |
| PARENT_SERVICE_CODE | SERVICE_CODE | | Yes | Parent Test Code |
| CHILD_SERVICE_PROVIDER_ID | SERVICE_PROVIDER_ID | | Yes | Child Provider ID |
| | | | | (In one row child |
| | | | | Provider Id and |
| | | | | Parent Provider |
| | | | | ID will always be |
| | | | | the same, the |
| | | | | provider ID has been |
| | | | | added for referential |
| | | | | integrity constraints) |
| CHILD_SERVICE_CODE | SERVICE_CODE | | | Child Test Code |
| CPT_MULTIPLIER | | Always 1 Not | No | |
| | | Used | | |
| SERVICE_PROFILE_VERSION | | Service | Yes | Versioning |
| | | Profile | | Information |
| | | Version | | |
| SORT_ORDER | | Indicates the | No | |
| | | order in | | |
| | | which the | | |
| | | Test Results | | |
| | | are to be | | |
| | | displayed in | | |
| | | the result | | |
| | | report | | |

TABLE SERVICE_PROVIDER
Service Provider table would have listing of NECLA labs.

| | | | | | | De- | | |
|---|---|---|---|---|---|---|---|---|
| | | | | | | scrip- | Need- | |
| Column Name | Constraint | Null? | Data type | FK Table | FK Column | tion | ed? | Comments |
| SERVICE_PROVIDER_ID | SERVICE_ORG | No | INTEGER | OR- | ORGANIZATION_ID | | Yes | NECLA |
| | | | | GANIZA- | | | | Labs ID |
| | | | | TION | | | | |
| SERVICE_PROVIDER_NAME | | No | VARCHAR(10) | | | | Yes | NECLA |
| | | | | | | | | Labs |
| | | | | | | | | Name |
| SERVICE_PROVIDER_TYPE | | No | VARCHAR(10) | | | Type | Yes | NECLA |
| | | | | | | of Lab | | Labs type |
| | | | | | | | | ??? |

TABLE SERVICE_ORDER

| Column Name | Constraint | Null? | Data type | FK Table | FK Column | Description | Needed? | Comments |
|---|---|---|---|---|---|---|---|---|
| SERVICE_ORDER_ID | | No | INTEGER | | | | Yes | Order Id, Need |
| | | | | | | | | to internally generate for |
| | | | | | | | | VDIS. LAB OrderID will be |
| | | | | | | | | sent along with Results from |
| | | | | | | | | FTP upload hospitals, HL7, Webforms |
| EVENT_ID | INCLUDES | No | INTEGER | PATIENT_EVENT | EVENT_ID | | Yes | Dummy Event |
| | | | | | | | | will be created for Patient |
| ORDERING_PROVIDER | PLACES | No | INTEGER | CONTACT | CONTACT_ID | | Yes | Defaulted to |
| | | | | | | | | PCP for patient |
| BILLING_PROVIDER | RESPONSIBLE_{——}FOR | | INTEGER | CONTACT | CONTACT_ID | | No | |
| ORDERING_CLIENT | ORDERS | No | INTEGER | CLIENT | CLIENT_ID | Ordering Site | Yes | Defaulted to |
| | | | | | | | | PCP for patient |
| | SO_ACCOUNT_FOR | | | ACCOUNT | CLIENT_ID | | | |
| ACCOUNT_NUMBER | | | VARCHAR(7) | | NUMBER | Account for | No billing | |
| REPORTABLE_COMM | | | VARCHAR(80) | | | Not Used | No | |
| NONREPORTABLE_COMM | | | VARCHAR(80) | | | Not Used | No | |
| STATUS | SO_STATUS_OF | | VARCHAR(10) | ORDER_LOOKUP | CODE | Status_code | No | |
| | | | | | | is the status | | |
| | | | | | | of the order. | | |
| | | | | | | One order | | |
| | | | | | | can have | | |
| | | | | | | many tests. | | |
| | | | | | | The status of | | |
| | | | | | | the Order is | | |
| | | | | | | the common | | |
| | | | | | | minimum | | |
| | | | | | | status of all | | |
| | | | | | | tests involved | | |
| | | | | | | in that order. | | |
| PLACER_ORDER_NUM | | | VARCHAR(20) | | | The | No | |
| | | | | | | Placer_Order_Num | | |
| | | | | | | is the PathLINE RL | | |
| | | | | | | order number. This | | |
| | | | | | | has the format | | |
| | | | | | | PAXXXXXX where X is | | |
| | | | | | | any number between 0 to 9. | | |
| FILLER_ORDER_NUM | | | VARCHAR(20) | | | Filer_Order_Num | No | Not being used |
| | | | | | | is | | by OCMS |
| | | | | | | currently not | | |
| | | | | | | been used | | |
| | | | | | | but it is | | |
| | | | | | | planned to be | | |
| | | | | | | used for | | |
| | | | | | | storing the | | |
| | | | | | | Performing | | |
| | | | | | | site Order | | |
| | | | | | | numbers, if | | |
| | | | | | | any. | | |
| ORDERING_LIS_ORDER_NUM | | | | | | Order | No | Site specific |
| | | | | | | Number | | order number. |
| | | | | | | created by | | Not of any use |
| | | | | | | the Ordering | | to us as we are |
| | | | | | | LIS. Column | | dealing only |
| | | | | | | to be added | | with Accession |
| | | | | | | to the | | Numbers |
| | | | | | | schema. Not | | |
| | | | | | | there yet | | |

Mandatory Master Tables:

| Table Name | Description |
|---|---|
| SERVICE_LOOKUP | Lookup for various codes used by Services |
| PROCEDURE | Stores the internal test codes |
| ORGANIZATION | Information about All organizations enrolled |
| | with VDIS |
| CONTACT | Contact information for individuals |

TABLE PATIENT

| Column Name | Constraint | Null? | Data type | FK Table | FK Column | Description | Needed? | Comments |
|---|---|---|---|---|---|---|---|---|
| PATIENT_ID | | No | INTEGER | | | Internal patient | Yes | Internal |
| | | | | | | identifier for VDIS | | patient identifier for VDIS |
| LAST_NAME | | No | VARCHAR(40) | | | Last Name | Yes | Self explanatory |
| FIRST_NAME | | No | VARCHAR(40) | | | First Name | Yes | Self explanatory |
| MIDDLE_NAME | | | VARCHAR(20) | | | Middle Name | Yes | Self explanatory |
| DOB | | No | DATE | | | Date of Birth | Yes | Self explanatory |
| PREFIX | | | VARCHAR(4) | | | Prefix | Yes | e.g. Mr, Mrs |
| SUFFIX | | | VARCHAR(10) | | | Suffix | Yes | e.g. Jr, Sr. |
| SPOUSE | | | VARCHAR(100) | | | No Used | No | |
| LAST_MESSAGE_ID | | | NUMBER(16) | | | Last Update for the | No | |
| | | | | | | patient. Set by the ADT Interface. | | |
| GUARANTOR | | | VARCHAR(100) | | | Not Used | No | |
| EMPLOYER_SCHOOL | | | VARCHAR(40) | | | Not Used | No | |
| PATIENT_HOME_LOCATION | HOUSES | | INTEGER | LOCATION | LOCATION_ID | Not Used | Yes | This needs |
| | | | | | | | | to be opulated to send out |
| | | | | | | | | mails. Location details in |
| | | | | | | | | Location table. |
| CURRENT_PROVIDER | CURRENT_PROVIDER | | INTEGER | CONTACT | CONTACT_ID | Physician attending the patient. | No | |
| | | | | | | May be the patient was referred. | | |
| | | | | | | This data is loaded by the ADT | | |
| | | | | | | interface from IDX. Not used by | | |
| | | | | | | OCMS | | |
| PRIMARY_PROVIDER | PCP_OF | | INTEGER | CONTACT | CONTACT_ID | Primary care | Yes | Primary care physician |
| | | | | | | for the patient. This data is | | for the patient. Refer |
| | | | | | | loaded by the ADT interface | | to CONTACT table. |
| | | | | | | from IDX. Not used by OCMS | | |
| DATE_OF_DEATH | | | DATE | | | Date of Death | Yes | Research |
| | | | | | | | | related data. |
| | | | | | | | | Self- |
| | | | | | | | | explanatory. |
| SEX | SEX_OF | No | VARCHAR(10) | PSEX_LOOKUP | CODE | Sex | Yes | Self- |
| | | | | | | | | explanatory |
| GUAR_RELATION | GRELATIONSHIP | | VARCHAR(15) | PGUAR_REL_LOOKUP | CODE | Relationship with Guarantor | No | |
| MARITAL_STATUS | MSTATUS_OF | | VARCHAR(10) | PMARITAL_LOOKUP | CODE | Marital Status | Yes | Research |
| | | | | | | | | related data. |
| | | | | | | | | Self-explanatory |
| ETHNIC_GROUP | PETHNIC | | VARCHAR(10) | PETHNIC_LOOKUP | CODE | Ethnic Group | Yes | Research |
| | | | | | | | | related data. |
| | | | | | | | | Self-explanatory |
| SPECIES | SPECIES_OF | | VARCHAR(10) | PSPECIES_LOOKUP | CODE | Species | No | |
| STATUS | PSTATUS_OF | | VARCHAR(10) | PSTATUS_LOOKUP | CODE | Not Used | No? | What is |
| | | | | | | | | OCMS using this column |
| | | | | | | | | for? |

TABLE
ALTERNATIVE_PATIENT_ID
This table is used to may the internal VDIS patient identifier to the site specific identifier

| Column Name | Constraint | Null? | Data Type | FK Table | FK Column | Description | Needed? | Comments |
|---|---|---|---|---|---|---|---|---|
| ORGANI- | IDS_PATIENTS_AS | No | INTEGER | ORGANI- | ORGANI- | Organi- | Yes | Organi- |
| ZATION_ID | | | | ZATION | ZATION_ID | zation ID | | zations can |
| | | | | | | | | be NECLA |
| | | | | | | | | at broader |
| | | | | | | | | level. |
| PATIENT_ID | ALSO_IDD_AS | No | INTEGER | PATIENT | PATIENT_ID | Internal | Yes | Internal |
| | | | | | | VDIS | | VDIS |
| | | | | | | identifier | | identifier |
| | | | | | | | | for |
| | | | | | | | | Patient |
| PATIENT_AL- | | No | VARCHAR(20) | | | Alternate | Yes | ID from |
| TERNATIVE_ID | | | | | | ID | | other |
| | | | | | | | | LAB |
| ALT_ID_DE- | ALT_ID_DESC_OF | No | VARCHAR(10) | ALT_PID_LOOK- | CODE | Description | Yes | Could be |
| SCRIPTION | | | | UP | | for ID | | SSN/MRN |

TABLE
PATIENT_EVENT

| Column Name | Constraint | Null? | Data Type | FK Table | FK Column | Description | Needed | Comments |
|---|---|---|---|---|---|---|---|---|
| EVENT_ID | | No | INTEGER | | | Event ID | Yes | Event ID |
| | | | | | | associated | | associated |
| | | | | | | with each | | with each |
| | | | | | | order | | order, |
| | | | | | | | | internally |
| | | | | | | | | generated |
| SCHEDULE_ID | EVENT_OF | | CHAR(10) | EVENT_SCHED- | SCHED- | Not Used | No, | |
| | | | | ULE | ULE_ID | | NULL | |
| PATIENT_ID | | No | INTEGER | | | Internal | Yes | VDIS |
| | | | | | | Id | | Patient ID |
| PCP_PROVIDER | DEFINES | | INTEGER | CONTACT | CON- | PCP | No | |
| | | | | | TACT_ID | | | |
| EVENT_TYPE | EVENT_TYPE_OF | | VARCHAR(10) | EVENT_LOOK | CODE | Type | No | |
| | | | | UP | | | | |
| PA- | | | TIMESTAMP | | | Order | No | |
| TIENT_EVENT_DATE | | | | | | Date | | |

Mandatory Master Tables:

| Table Name | Description |
|---|---|
| ORGANIZATION | Information about All organizations |
| | enrolled with VDIS |
| CONTACT | Contact information for individuals |
| LOCATION | Address information for patients or contacts |

Population of Miscellaneous Tables

This section outlines the “non master” tables that are needed for the population of Patient, Service or Result tables.

TABLE
CONTACT
This is the master list of all the providers (Physicians) for VDIS

| Column Name | Constraint | Null? | Data Type | FK Table | FK Column | Description | Needed | Comments |
|---|---|---|---|---|---|---|---|---|
| CONTACT_ID | | No | INTEGER | | | Internal ID | Yes | VDIS Internal ID for |
| | | | | | | | | Physician |
| LAST_NAME | | | VARCHAR(40) | | | | Yes | Self-explanatory |
| FIRST_NAME | | | VARCHAR(40) | | | | Yes | Self-explanatory |
| MIDDLE_NAME | | | VARCHAR(20) | | | | Yes | Self-explanatory |
| PREFIX | | | VARCHAR(4) | | | | Yes | Mr, Mrs |
| SUFFIX | | | VARCHAR(15) | | | | Yes | Jr., Sr. |
| TITLE | | | VARCHAR(50) | | | | Yes | MD, Nurse etc. |
| WORK_PHONE | | | VARCHAR(14) | | | | Yes | Work Phone |
| HOME_PHONE | | | VARCHAR(14) | | | | Yes | Home Phone |
| MOBILE_PHONE | | | VARCHAR(14) | | | | Yes | Mobile Number |
| PAGER | | | VARCHAR(14) | | | | Yes | Pager Number |
| FAX | | | VARCHAR(14) | | | | Yes | Fax Number |
| EMAIL | | | VARCHAR(50) | | | | Yes | Email |
| NOTE | | | VARCHAR(50) | | | | No | |
| USERNAME | | | VARCHAR(15) | | | User ID | Yes | Can be linked to User |
| | | | | | | | | Admin tables. To |
| | | | | | | | | configure user privileges |
| PIN | | | VARCHAR(6) | | | Personal | Yes | Can be linked to User |
| | | | | | | Identifier | | Admin tables as |
| | | | | | | | | password. To configure |
| | | | | | | | | user privileges |
| DEBUG_LEVEL | | | INTEGER | | | | No | |
| SECURITY_CONTEXT | | | VARCHAR(15) | | | | No | |

TABLE
ALTERNATE_CONTACT_ID
This table is needed as a part of the initial upload as well as the report upload.
All sites would provide information in terms of their internal contact ID's. This
needs to be cross-referenced to map to the internal id

| Column Name | Constraint | Null? | Data Type | FK Table | FK Column | Description | Needed | Comments |
|---|---|---|---|---|---|---|---|---|
| ORGANI- | CONTACT_ID_OF | No | INTEGER | ORGANI- | ORGANI- | Internal | Yes | |
| ZATION_ID | | | | ZATION | ZATION_ID | Organization | | |
| | | | | | | Identifier | | |
| CONTACT_ID | CONTACT_ID_FOR | No | INTEGER | CONTACT | CONTACT_ID | Internal | Yes | |
| | | | | | | Contact | | |
| | | | | | | Identifier | | |
| ID_TYPE | TYPE_OF_ID | No | VARCHAR(10) | CON- | CODE | Type of ID, | Yes | |
| | | | | TACT_LOOKUP | | perhaps | | |
| | | | | | | SSN/MRN | | |
| ID_VALUE | | No | VARCHAR(25) | | | Site | Yes | |
| | | | | | | Specific ID | | |
| ACTI- | | | DATE | | | | | |
| VATION_DATE | | | | | | | | |
| DEACTI- | | | DATE | | | | | |
| VATION_DATE | | | | | | | | |
| STATUS | CONTACT_STA- | | VARCHAR(10) | CON- | CODE | | Yes | |
| | TUS_OF | | | TACT_LOOKUP | | | | |

TABLE
CLIENT
This table stores the list of possible clients of VDIS. It is a subset of Organization.
This table would store the Practice specific information for VDIS.

| Column Name | Constraint | Null? | Data Type | FK Table | FK Column | Description | Needed? | Comments |
|---|---|---|---|---|---|---|---|---|
| CLIENT_ID | CLI- | No | INTEGER | ORGANI- | ORGANI- | Internal ID | Yes | Practice |
| | ENT_ORG | | | ZATION | ZATION_ID | | | ID |
| NAME | | | VARCHAR(40) | | | Name of | Yes | Practice |
| | | | | | | Client | | Name |
| STA- | | | DATE | | | Not Used | No | |
| TUS_CHANGE_DATE | | | | | | | | |
| ADDED_DATE | | | VARCHAR(10) | | | Create Date | Yes | When |
| | | | | | | | | Practice is |
| | | | | | | | | added to |
| | | | | | | | | study |
| NUM- | | | INTEGER | | | Number of | No | |
| BER_OF_LABELS | | | | | | copies of | | |
| | | | | | | labels to | | |
| | | | | | | be printed. | | |
| | | | | | | Not used | | |
| VISIT_NOTE | | | VARCHAR(10) | | | Not used | No | |
| CONTACT_ID | SER- | | INTEGER | CONTACT | CON- | Single | Yes | Primary |
| | VICES_CLI- | | | | TACT_ID | Point of | | contact for |
| | ENT | | | | | contact | | Practice. |
| | | | | | | for the | | |
| | | | | | | client | | |
| STATUS | CLI- | | VARCHAR(10) | CLI- | CODE | Status | Yes | The column |
| | ENT_STA- | | | ENT_LOOKUP | | | | could |
| | TUS_OF | | | | | | | potentially |
| | | | | | | | | store |
| | | | | | | | | whether |
| | | | | | | | | the “client” |
| | | | | | | | | is currently |
| | | | | | | | | in the |
| | | | | | | | | active group |
| | | | | | | | | or in the |
| | | | | | | | | control |
| | | | | | | | | group |
| TYPE | CLI- | | VARCHAR(10 | CLI- | CODE | Not Used | Yes | ??? |
| | ENT_TYPE_OF | | | ENT_LOOKUP | | | | |

TABLE
WORKS_FOR_EMPLOYS
Identifies which Physician is working for which Practice.

| | | | | | | Descrip- | | |
|---|---|---|---|---|---|---|---|---|
| Column Name | Constraint | Null? | Data Type | FK Table | FK Column | tion | Needed? | Comments |
| CLIENT_ID | EMPLOYS | No | INTEGER | ORGANI- | ORGANI- | Internal | Yes | VDIS Internal |
| | | | | ZATION | ZATION_ID | ID | | ID, Refer to |
| | | | | | | | | ORGANIZATION |
| | | | | | | | | table |
| CONTACT_ID | WORKS_FOR | | INTEGER | CONTACT | CON- | Name of | Yes | VDIS Internal |
| | | | | | TACT_ID | Client | | ID, Refer to |
| | | | | | | | | CONTACT |
| | | | | | | | | table |
| EMPLOY- | | | DATE | | | Not Used | No | |
| MENT_DATE | | | | | | | | |
| TERMINA- | | | DATE | | | Create | No | |
| TION_DATE | | | | | | Date | | |
| STATUS | WFE_STA- | | VARCHAR(10) | CON- | CODE | Status | No | |
| | TUS_OF | | | TACT_LOOKUP | | | | |

TABLE
ORGANIZATION
This table stores the details of all the sites

| Column Name | Constraint | Null? | Data Type | FK Table | FK Column | Description | Needed ? | Comments |
|---|---|---|---|---|---|---|---|---|
| ORGANI- | | No | INTEGER | | | Internal ID | Yes | VDIS Internal |
| ZATION_ID | | | | | | | | ID, e.g. NECLA |
| ORGANI- | | No | VARCHAR(32) | | | Name | Yes | Name of |
| ZATION_NAME | | | | | | | | Organization |
| LOCA- | ORG_LOCA- | | INTEGER | LOCATION | LOCA- | Address | Yes | Address info for |
| TION_ID | TION | | | | TION_ID | Info. | | Organization |
| IS_CLIENT | | | VARCHAR(1) | | | Is Client? | Yes | Is Practice? |
| IS_SER- | | | VARCHAR(1) | | | Is | Yes | Is Lab? |
| VICE_PRO- | | | | | | Provider? | | |
| VIDER | | | | | | | | |
| IS_PARENT | | | VARCHAR(1) | | | Is Parent? | Yes | ??? |
| IS_NETWORK | | | VARCHAR(1) | | | Is | Yes | Is on VDIS |
| | | | | | | Network? | | network??? |

TABLE
SENDING_APP
This table maps the Sending Application Name to the Organization ID. This would act
as a lookup table when reports are uploaded using FTP.

| Column Name | Constraint | Null? | Data Type | FK Table | FK Column | Description | Needed ? | Comments |
|---|---|---|---|---|---|---|---|---|
| APP_NAME | | No | VARCHAR(10) | | | Sending | Yes | Sending Lab |
| | | | | | | Application | | name |
| | | | | | | Name | | |
| ORGANIZATION_ID | | No | INTEGER | ORGANI- | ORGANI- | Organization | Yes | Sending Lab |
| | | | | ZATION | ZATION_ID | | | ID for VDIS |

TABLE
RELATED_TO
It is self-referencing table

| Column Name | Constraint | Null? | Data Type | FK Table | FK Column | Description | Needed ? | Comments |
|---|---|---|---|---|---|---|---|---|
| ORGANIZATION_ID_1 | | No | INTEGER | | | Parent | Yes | Say FAHC is |
| | | | | | | Organization | | group of Labs |
| ORGANIZATION_ID_2 | | No | INTEGER | | | Child | Yes | FAHC lab_1 |
| | | | | | | Organization | | |
| RELATED_TO_RANK | ORG_LOCATION | | INTEGER | LOCATION | LOCA- | | Yes | Its location |
| | | | | | TION_ID | | | |
| RELATED_TO_TYPE | | No | VARCHAR(10) | | | Type of | Yes | ??? |
| | | | | | | relation | | |

TABLE
LOCATION
This table stored location information for clients, contacts and organizations

| | | | | | | | | |
|---|---|---|---|---|---|---|---|---|
| | | | | FK | | | | |
| Column Name | Constraint | Null? | Data Type | Table | FK Column | Description | Needed? | Comments |

| | | | | | |
|---|---|---|---|---|---|
| LOCATION_ID | No | INTEGER | Internal | Yes | VDIS internal ID |
| | | | sequence | | |
| NAME | | VARCHAR(40) | | Yes | Self-explanatory |
| | | | | | Not Used |
| ADDRESS_1 | | VARCHAR(50) | | Yes | Self-explanatory |
| ADDRESS_2 | | VARCHAR(50) | | Yes | Self-explanatory |
| CITY | | VARCHAR(20) | | Yes | Self-explanatory |
| STATE | | VARCHAR(2) | | Yes | Self-explanatory |
| ZIP | | VARCHAR(9) | | Yes | Self-explanatory |
| COUNTRY | | VARCHAR(15) | | Yes | Self-explanatory |
| PHONE | | VARCHAR(10) | | Yes | Self-explanatory |
| PHONE_2 | | VARCHAR(10) | | Yes | Self-explanatory |
| FAX | | VARCHAR(10) | | Yes | Self-explanatory |
| HOURS | | VARCHAR(30) | Not used | No | |
| SUPPLY_DIST | | VARCHAR(15) | Not used | No | |

VDIS Specific Tables
This section outlines additional tables that would be added to maintain VDIS specific data.

Master Tables

TABLE
PATIENT_STATUS_LOOKUP
This table stores VDIS specific patient status information.

| | | | | | | | | |
|---|---|---|---|---|---|---|---|---|
| | | | | FK | FK | | | |
| Column Name | Constraint | Null? | Data Type | Table | Column | Description | Needed? | Comments |

| | | | | | |
|---|---|---|---|---|---|
| PATIENT_STATUS_ID | No | NUMBER | Identifier | Yes | Internal Identifier |
| PATIENT_STATUS | | VARCHAR2(20) | Patient Status | Yes | Short text for the status |
| | | | Description | | |

TABLE
REPORT_LOOKUP
This table stores the list of VDIS specific reports

| | | | | | | | | |
|---|---|---|---|---|---|---|---|---|
| | | | | FK | | | | |
| Column Name | Constraint | Null? | Data Type | Table | FK Column | Description | Needed? | Comments |

| | | | | | |
|---|---|---|---|---|---|
| REPORT_ID | No | NUMBER | Identifier | Yes | Internal Identifier |
| REPORT | | VARCHAR2(20) | Report Name | Yes | Type of Report |

TABLE
PARSER_CLASS_LOOKUP
This table would store the list of parser classes available for parsing upload data.

| | | | | | | | | |
|---|---|---|---|---|---|---|---|---|
| | | | | FK | | | | |
| Column Name | Constraint | Null? | Data Type | Table | FK Column | Description | Needed? | Comments |

| | | | | | |
|---|---|---|---|---|---|
| PARSER_CLASS_ID | No | NUMBER | Identifier | Yes | Internal Identifier |
| PARSER_CLASS | | VARCHAR2(20) | Parser Name | Yes | Fully qualified Java class |
| | | | | | name |

TABLE
FILE_STATUS_LOOKUP
This table would store the possible processing statuses of the uploaded FTP files

| | | | | | | | | |
|---|---|---|---|---|---|---|---|---|
| | | | | FK | | | | |
| Column Name | Constraint | Null? | Data Type | Table | FK Column | Description | Needed? | Comments |

| | | | | | |
|---|---|---|---|---|---|
| FILE_STATUS_ID | No | NUMBER | Identifier | Yes | Internal Identifier |
| FILE_STATUS | | VARCHAR2(50) | File Status | Yes | Short text for the File |
| | | | | | Status |

TABLE
MODULE_LOOKUP
This table would store the list of modules in the system.

| | | | | | | | | |
|---|---|---|---|---|---|---|---|---|
| | | | | FK | | | | |
| Column Name | Constraint | Null? | Data Type | Table | FK Column | Description | Needed? | Comments |

| | | | | | |
|---|---|---|---|---|---|
| MODULE_ID | No | NUMBER | Identifier | Yes | Internal Identifier |
| MODULE | | VARCHAR2(20) | Module Name | Yes | Module Name |

TABLE
OPERATION_TYPE_LOOKUP
This table would store the type of operations

| | | | | | | | | |
|---|---|---|---|---|---|---|---|---|
| | | | | FK | | | | |
| Column Name | Constraint | Null? | Data Type | Table | FK Column | Description | Needed? | Comments |

| | | | | | |
|---|---|---|---|---|---|
| OPERATION_TYPE_ID | No | NUMBER | Identifier | Yes | Internal Identifier |
| OPERATION_TYPE | | VARCHAR2(20) | Operation Type | Yes | Operation Type |

TABLE
REPORT_STATUS_LOOKUP
This table would store the report statuses (faxed, printed, generated)

| | | | | | | | | |
|---|---|---|---|---|---|---|---|---|
| | | | | FK | | | | |
| Column Name | Constraint | Null? | Data Type | Table | FK Column | Description | Needed? | Comments |

| | | | | | |
|---|---|---|---|---|---|
| REPORT_STATUS_ID | No | NUMBER | Identifier | Yes | Internal Identifier |
| REPORT_STATUS | | VARCHAR2(20) | Status of | Yes | Operation Type |
| | | | Report | | |

TABLE
OUTPUT_TYPE_LOOKUP
This table would store the output types e.g. Fax, Print

| | | | | | | | | |
|---|---|---|---|---|---|---|---|---|
| | | | | FK | | | | |
| Column Name | Constraint | Null? | Data Type | Table | FK Column | Description | Needed? | Comments |

| | | | | | |
|---|---|---|---|---|---|
| OUTPUT_TYPE_ID | No | NUMBER | Identifier | Yes | Internal Identifier |
| OUTPUT_TYPE_DESC | | VARCHAR2(10) | Type of output | Yes | Fax, Print, File |

TABLE
CANNED_TEXT_LOOKUP
This is the master table, which contains the different canned texts to be used in all reports and their associated Ids.

| | | | | | | | | |
|---|---|---|---|---|---|---|---|---|
| | | | | FK | FK | | | |
| Column Name | Constraint | Null? | Data Type | Table | Column | Description | Needed? | Comments |

| | | | | | |
|---|---|---|---|---|---|
| CANNED_TEXT_ID | No | NUMBER | Identifier | Yes | Internal Identifier |
| CANNED_TEXT | | VARCHAR2(256) | Canned Content | Yes | Canned Text Content |

Detail Tables

TABLE
REPORT_LOG
This table is the master table, which contains the different canned texts to be used in all reports and their associated Ids.

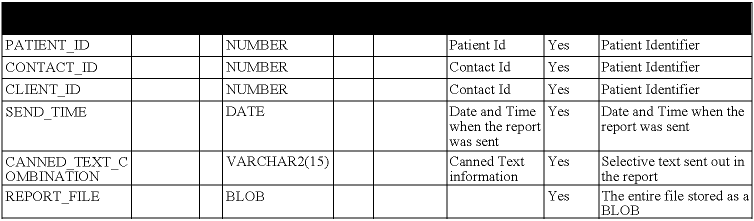

TABLE
PATIENT_STATUS_CHANGE_HISTORY
This table stores information regarding the status change information for a patient

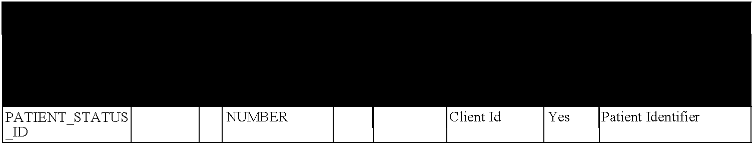

TABLE
FILE_DATA
This table stores the file processing status

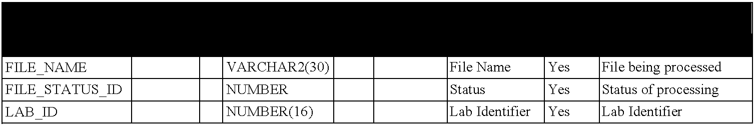

TABLE
FTP_CONFIG
This table stores the FTP configuration information.

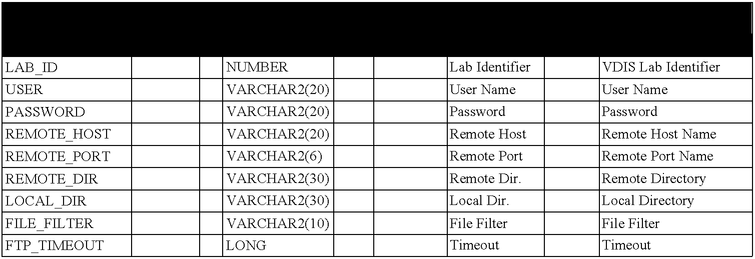

TABLE
LIS_DATA
This table stores the LIS specific information

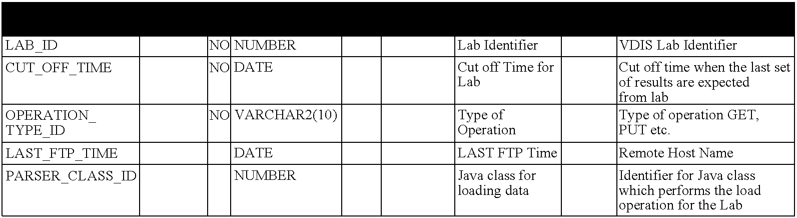

TABLE
GRACE_PERIOD
This table stores the grace period information for a client's tests.

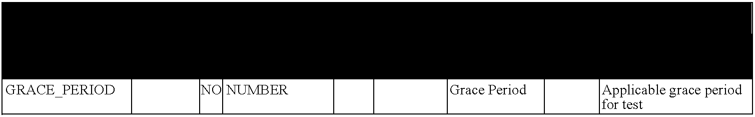

TABLE
DIABETES_TEST_REF_RANGE
This table stores the reference range information for a client's tests

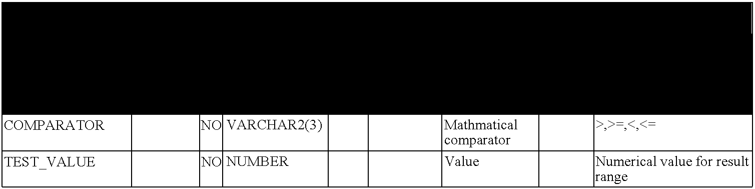

TABLE
CLIENT_DATA
This table stores client specific data

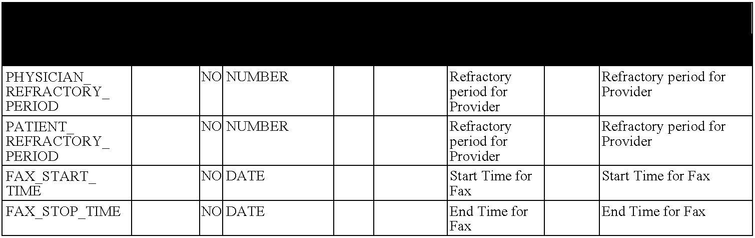

TABLE
LAST_REPORT_SENT
Information about the Reminders sent out to as well as the date when a microalbumin alert was sent (if any)

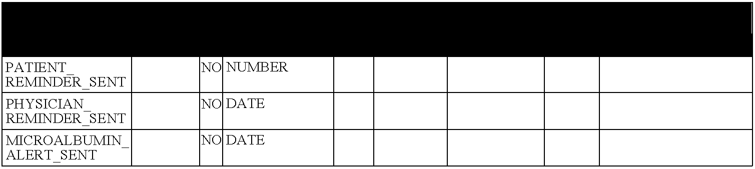

TABLE
TEST_GENERATION_LOG
Information about the Reminders alerts out for every result

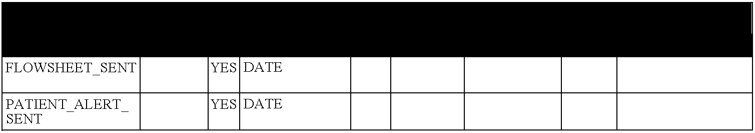

TABLE
FS_LIPID_TRUTH_TABLE
This is the truth table for the Lipid Flow Sheet canned text generation

| | | | | FK | FK | | | |
|---|---|---|---|---|---|---|---|---|
| Column Name | Constraint | Null? | Data Type | Table | Column | Description | Needed? | Comments |
| LDL_RESULT_RANGE_TYPE | | No | VARCHAR2(10) | | | LDL Result | | LDL Result Range |
| | | | | | | Range | | |
| TRIG_RESULT_RANGE_TYPE | | No | VARCHAR2(10) | | | TRIG Result | | TRIG Result Range |
| | | | | | | Range | | |
| OVERDUE_FLAG | | No | VARCHAR2(1) | | | Overdue Status | | Overdue Status |
| SEQUENCE_NO | | No | NUMBER | | | Sequence | | Sequence Number which |
| | | | | | | number for | | determines order in which |
| | | | | | | Canned text ID | | the canned text needs to |
| | | | | | | | | be used |
| CANNED_TEXT_ID | | No | NUMBER | | | Canned Text ID | | Canned Text ID |

TABLE
FS_A1C_TRUTH_TABLE
This is the truth table for the A1C Flow Sheet canned text selection.

| | Con- | | | FK | FK | | | |
|---|---|---|---|---|---|---|---|---|
| Column Name | straint | Null? | Data Type | Table | Column | Description | Needed? | Comments |
| RESULT_RANGE_TYPE | | No | VARCHAR2(10) | | | LDL Result | | Result range type |
| | | | | | | Range | | |
| OVERDUE_FLAG | | No | VARCHAR2(1) | | | Overdue Status | | Overdue Status |
| SEQUENCE_NO | | No | NUMBER | | | Sequence | | Sequence Number |
| | | | | | | number for | | which determines |
| | | | | | | Canned text ID | | order in which |
| | | | | | | | | the canned text |
| | | | | | | | | needs to be used |
| CANNED_TEXT_ID | | No | NUMBER | | | Canned Text ID | | Canned Text ID |

TABLE
FS_MC_TRUTH_TABLE
This is the truth table for the Microalbumin and Creatinine Flow Sheet canned text selection.

| | | | | FK | FK | | | |
|---|---|---|---|---|---|---|---|---|
| Column Name | Constraint | Null? | Data Type | Table | Column | Description | Needed? | Comments |
| TEST_TYPE | | No | VARCHAR2(10) | | | Test Type | | Test Type |
| LATEST_RESULT_RANGE_TYPE | | No | VARCHAR2(10) | | | Latest Result | | Latest Result range |
| | | | | | | range for MCR | | for MCR |
| PREVIOUS_RESULT_RANGE_TYPE | | No | VARCHAR2(10) | | | Previous Result | | Previous Result |
| | | | | | | range for MCR | | range for MCR |
| OVERDUE_FLAG | | No | VARCHAR2(1) | | | Overdue Status | | Overdue Status |
| SEQUENCE_NO | | No | NUMBER | | | Sequence | | Sequence Number |
| | | | | | | number for | | which determines |
| | | | | | | Canned text ID | | order in which the |
| | | | | | | | | canned text needs |
| | | | | | | | | to be used |
| CANNED_TEXT_ID | | No | NUMBER | | | Canned Text ID | | Canned Text ID |

TABLE
REMINDER_TRUTH_TABLE
This is the common truth table for the patient and physician truth tables for canned text selection. Similar to the Flow Sheet
truth tables, this table only has a extra column to identify whether the reminder is a physician reminder or patient reminder.

| | | | | FK | FK | | | |
|---|---|---|---|---|---|---|---|---|
| Column Name | Constraint | Null? | Data Type | Table | Column | Description | Needed? | Comments |
| REMINDER_TYPE | | No | VARCHAR2(10) | | | Test Type | | Reminder Type |
| TEST_TYPE | | No | VARCHAR2(10) | | | Latest Result | | Test Type |
| | | | | | | range | | |
| RESULT_RANGE_TYPE | | No | VARCHAR2(10) | | | Previous Result | | Result range |
| | | | | | | range | | |
| SEQUENCE_NO | | No | NUMBER | | | Sequence | | Sequence Number |
| | | | | | | number for | | which determines |
| | | | | | | Canned text ID | | order in which the |
| | | | | | | | | canned text |
| | | | | | | | | needs to be used |
| CANNED_TEXT_ID | | No | NUMBER | | | Canned Text ID | | Canned Text ID |

TABLE
DIABETES_TEST_OVERDUE_PERIODS
This table stores the overdue periods for the different combinations of TEST_TYPEs (i.e. HgbA1C, Microalbumin, creatinine etc.),
REPORT_TYPEs (i.e. flowsheet/reminder), RESULT_RANGEs (i.e. high, medium, low). Thus all the overdue periods are stored
in this single table.

| | | | | FK | FK | | | |
|---|---|---|---|---|---|---|---|---|
| Column Name | Constraint | Null? | Data Type | Table | Column | Description | Needed? | Comments |
| TEST_TYPE | | No | VARCHAR2(10) | | | Latest Result | | Test Type |
| | | | | | | range | | |
| REPORT_TYPE | | No | VARCHAR2(10) | | | Flowsheet or | | Report Type |
| | | | | | | Reminder | | |
| RESULT_RANGE_TYPE | | No | VARCHAR2(10) | | | Previous Result | | Result range |
| | | | | | | range | | |
| OVERDUE_PERIOD | | No | NUMBER | | | Overdue period | | Overdue period in days. |
| | | | | | | in days | | |

Remaining tables
The remaining tables below store patient, account, additional test and order tables. Views are also listed below.
Listing tables individually and detail comments about each field will follow in the next version of this document.

| | | | | | FK |
|---|---|---|---|---|---|
| Table Name | Column Name | Constraint | Null? | Data Type | Table |
| ACCOUNT | ACCOUNT_ID | | | NUMBER | |
| ACCOUNT | CONTRACT_STARTDATE | | | DATE | |
| ACCOUNT | CONTRACT_ENDDATE | | | DATE | |
| ACCOUNT | NAME | | | VARCHAR2 | |
| ACCOUNT | DESCRIPTION | | | VARCHAR2 | |
| ACCOUNT | REPORT_DISPLAY_NAME1 | | | VARCHAR2 | |
| ACCOUNT | REPORT_DISPLAY_NAME2 | | | VARCHAR2 | |
| ACCOUNT | CONTACT_ID | | | NUMBER | |
| ACCOUNT | ACCOUNT_TYPE | | | NUMBER | |
| ALT_PID_LOOKUP | | | | | |
| ALT_PID_LOOKUP | CODE | | | VARCHAR2 | |
| ALT_PID_LOOKUP | DESCRIPTION | | | VARCHAR2 | |
| ALT_PID_LOOKUP | ACTIVE | | | VARCHAR2 | |
| ALT_PID_LOOKUP | SORT_ORDER | | | NUMBER | |
| CLIENT_LOOKUP | CODE | | | VARCHAR2 | |
| CLIENT_LOOKUP | DESCRIPTION | | | VARCHAR2 | |
| CLIENT_LOOKUP | CLIENT_TYPE | | | VARCHAR2 | |
| CLIENT_LOOKUP | ACTIVE | | | VARCHAR2 | |
| CLIENT_LOOKUP | SORT_ORDER | | | NUMBER | |
| CODED_COLLECT_NOTE | COLLECT_NOTE | | | VARCHAR2 | |
| CODED_COLLECT_NOTE | NOTE_TEXT | | | CLOB | |
| CODED_COLLECT_NOTE | SERVICE_PROVIDER_ID | | | NUMBER | |
| CODED_COLLECT_NOTE | NOTE_TYPE | | | VARCHAR2 | |
| CONTACT_LOOKUP | CODE | | | VARCHAR2 | |
| CONTACT_LOOKUP | DESCRIPTION | | | VARCHAR2 | |
| CONTACT_LOOKUP | CONTACT_TYPE | | | VARCHAR2 | |
| CONTACT_LOOKUP | ACTIVE | | | VARCHAR2 | |
| CONTACT_LOOKUP | SORT_ORDER | | | NUMBER | |
| CONTROL_LIMIT | CONTROL_LIMIT_ID | | | NUMBER | |
| CONTROL_LIMIT | TYPE1 | | | VARCHAR2 | |
| CONTROL_LIMIT | TYPE2 | | | VARCHAR2 | |
| CONTROL_LIMIT | CONTROL_LIMIT_NAME | | | VARCHAR2 | |
| CONTROL_LIMIT | DESCRIPTION | | | VARCHAR2 | |
| CONTROL_LIMIT_DATA | CONTROL_LIMIT_ID | | | NUMBER | |
| CONTROL_LIMIT_DATA | DAY_OF_WEEK | | | NUMBER | |
| CONTROL_LIMIT_DATA | LOWER_CONTROL_LIMIT | | | NUMBER | |
| CONTROL_LIMIT_DATA | UPPER_CONTROL_LIMIT | | | NUMBER | |
| CPT | CPT_CODE | | | VARCHAR2 | |
| CPT | DESCRIPTION | | | VARCHAR2 | |
| CPT | EFFECTIVE_DATE | | | DATE | |
| DAYS_PERF_LOOKUP | DAYS_PERFORMED | | | VARCHAR2 | |
| DIABETES_TEST_OVERDUE_PERIODS | TEST_TYPE | | | VARCHAR2 | |
| DIABETES_TEST_OVERDUE_PERIODS | RESULT_RANGE | | | VARCHAR2 | |
| DIABETES_TEST_OVERDUE_PERIODS | REPORT_TYPE | | | VARCHAR2 | |
| DIABETES_TEST_OVERDUE_PERIODS | OVERDUE_PERIOD | | | NUMBER | |
| DIABETES_TEST_REF_RANGE | LAB_ID | | | NUMBER | |
| DIABETES_TEST_REF_RANGE | TEST_ID | | | VARCHAR2 | |
| DIABETES_TEST_REF_RANGE | LOW | | | FLOAT | |
| DIABETES_TEST_REF_RANGE | HIGH | | | FLOAT | |
| DIABETES_TEST_REF_RANGE | MEDIUM | | | VARCHAR2 | |
| DIABETES_TEST_REF_RANGE | LOW_INCLUSIVE | | | VARCHAR2 | |
| DIABETES_TEST_REF_RANGE | HIGH_INCLUSIVE | | | VARCHAR2 | |
| DIABETES_TEST_REF_RANGE | TEST_VERSION | | | VARCHAR2 | |
| ERROR_MESSAGES | ERROR_KEY | | | VARCHAR2 | |
| ERROR_MESSAGES | ERROR_MESSAGE | | | VARCHAR2 | |
| EVENT_LOOKUP | CODE | | | VARCHAR2 | |
| EVENT_LOOKUP | DESCRIPTION | | | VARCHAR2 | |
| EVENT_LOOKUP | EVENT_TYPE | | | VARCHAR2 | |
| EVENT_LOOKUP | ACTIVE | | | VARCHAR2 | |
| EVENT_LOOKUP | SORT_ORDER | | | NUMBER | |
| FS_MC_TRUTH_TABLE | TEST_TYPE | | | VARCHAR2 | |
| FS_MC_TRUTH_TABLE | PREVIOUS_RESULT_RANGE | | | VARCHAR2 | |
| FS_MC_TRUTH_TABLE | LATEST_RESULT_RANGE | | | VARCHAR2 | |
| FS_MC_TRUTH_TABLE | OVERDUE_FLAG | | | VARCHAR2 | |
| FS_MC_TRUTH_TABLE | TEXT_SEQUENCE | | | NUMBER | |
| FS_MC_TRUTH_TABLE | CANNED_TEXT_IDS | | | NUMBER | |
| FTP_FILE_DATA | FILE_DATA_ID | | | NUMBER | |
| FTP_FILE_DATA | FILE_NAME | | | VARCHAR2 | |
| FTP_FILE_DATA | FILE_STATUS | | | VARCHAR2 | |
| FTP_FILE_DATA | LAB_ID | | | NUMBER | |
| FTP_FILE_DATA | FILE_TYPE | | | NUMBER | |
| FTP_FILE_DATA | LOGGER_ID | | | NUMBER | |
| FTP_FILE_DATA | TIMESTAMP | | | DATE | |
| FTP_FILE_FIELD_MAPPING | LAB_ID | | | NUMBER | |
| FTP_FILE_FIELD_MAPPING | FIELD_NAME | | | VARCHAR2 | |
| FTP_FILE_FIELD_MAPPING | POSITION | | | NUMBER | |
| FTP_FILE_FIELD_MAPPING | REQUIRED | | | VARCHAR2 | |
| FTP_FILE_FIELD_MAPPING | DEFAULT_VALUE | | | VARCHAR2 | |
| FTP_FILE_FIELD_MAPPING | ACTIVE | | | VARCHAR2 | |
| FTP_FILE_FIELD_MAPPING | FORMAT | | | VARCHAR2 | |
| FTP_FILE_FIELD_MAPPING | FIELD_TYPE | | | VARCHAR2 | |
| GROUP_ACCESS | GROUP_NAME | | | VARCHAR2 | |
| GROUP_ACCESS | WEB_PAGE_URL | | | VARCHAR2 | |
| GROUP_ACCESS | APPLICATION_FLAG | | | VARCHAR2 | |
| GROUP_PRIVILEGE | CLIENT_ID | | | NUMBER | |
| GROUP_PRIVILEGE | CONTACT_ID | | | NUMBER | |
| GROUP_PRIVILEGE | GROUP_NAME | | | VARCHAR2 | |
| GROUP_PRIVILEGE | APPLICATION_FLAG | | | VARCHAR2 | |
| HAS_ROLE | ROLE_ID | | | VARCHAR2 | |
| HAS_ROLE | CONTACT_ID | | | NUMBER | |
| HISTORY_LOG | DATETIME | | | DATE | |
| HISTORY_LOG | PATIENT | | | VARCHAR2 | |
| HISTORY_LOG | PHYSICIAN | | | VARCHAR2 | |
| HISTORY_LOG | PRACTICE | | | VARCHAR2 | |
| HISTORY_LOG | ERRCODE | | | NUMBER | |
| HISTORY_LOG | ERRMSG | | | VARCHAR2 | |
| HISTORY_LOG | LOCATION | | | VARCHAR2 | |
| HISTORY_LOG | LAB | | | VARCHAR2 | |
| HISTORY_LOG | TEST_RESULT | | | VARCHAR2 | |
| ICD9 | ICD9_CODE | | | VARCHAR2 | |
| ICD9 | SHORT_DESCRIPTION | | | VARCHAR2 | |
| ICD9 | LONG_DESCRIPTION | | | VARCHAR2 | |
| ICD9 | ICD9_SPECIFICITY | | | VARCHAR2 | |
| ICD9_NEVER_COVERS | INSURANCE_PLAN_ID | | | NUMBER | |
| ICD9_NEVER_COVERS | COVERAGE_POLICY_ID | | | NUMBER | |
| ICD9_NEVER_COVERS | ICD9_CODE | | | VARCHAR2 | |
| ICD9_NEVER_COVERS | COVERAGE_COMMENT_CODE | | | VARCHAR2 | |
| ICD9_NEVER_COVERS | NC_EFFECTIVE_DATE | | | DATE | |
| ICD9_NEVER_COVERS | NC_ACTIVE_STATUS | | | VARCHAR2 | |
| ICD9_PARENT_CODE | ICD9_CODE | | | VARCHAR2 | |
| ICD9_PARENT_CODE | SHORT_DESCRIPTION | | | VARCHAR2 | |
| ICD9_PARENT_CODE | LONG_DESCRIPTION | | | VARCHAR2 | |
| ICD9_PARENT_CODE | ICD9_SPECIFICITY | | | VARCHAR2 | |
| ICD9_PARENT_CODE | PARENT_CODE | | | VARCHAR2 | |
| KEYMASTER | KEYMASTER_TABLE | | | VARCHAR2 | |
| KEYMASTER | KEYMASTER_KEY | | | NUMBER | |
| LOGGER | LOGGER_ID | | | NUMBER | |
| LOGGER | LOGGER_TIMESTAMP | | | DATE | |
| LOGGER | SEVERITY | | | VARCHAR2 | |
| LOGGER | LOGGER_SOURCE | | | VARCHAR2 | |
| LOGGER | DETAILED_SOURCE | | | VARCHAR2 | |
| LOGGER | DESCRIPTION | | | VARCHAR2 | |
| LOGGER | HL7_MESSAGE_ID | | | NUMBER | |
| LOGGER | HL7_MESSAGE_TYPE | | | VARCHAR2 | |
| LOGGER | LOGGER_STATUS | | | VARCHAR2 | |
| LOGGER | RESTART_POINT | | | VARCHAR2 | |
| LOGGER | CONTROL_ID | | | VARCHAR2 | |
| LOGGER_LOOKUP | CODE | | | VARCHAR2 | |
| LOGGER_LOOKUP | DESCRIPTION | | | VARCHAR2 | |
| LOGGER_LOOKUP | ACTIVE | | | VARCHAR2 | |
| LOGGER_LOOKUP | LOGGER_LOOKUP_TYPE | | | VARCHAR2 | |
| LOGGER_LOOKUP | SORT_ORDER | | | NUMBER | |
| LOGGER_MONITOR_CONTROL | LAST_MESSAGE_SENT | | | DATE | |
| LOGIN_VERIFICATION | CONTACT_ID | | | NUMBER | |
| LOGIN_VERIFICATION | PASSWORD_EXPIRY_DATE | | | DATE | |
| LOGIN_VERIFICATION | IS_ACTIVE | | | VARCHAR2 | |
| LOGIN_VERIFICATION | CURRENT_PASSWORD | | | VARCHAR2 | |
| LOGIN_VERIFICATION | PREV_PASSWORD_1 | | | VARCHAR2 | |
| LOGIN_VERIFICATION | PREV_PASSWORD_2 | | | VARCHAR2 | |
| LOGIN_VERIFICATION | PREV_PASSWORD_3 | | | VARCHAR2 | |
| LOGIN_VERIFICATION | PREV_PASSWORD_4 | | | VARCHAR2 | |
| LOGIN_VERIFICATION | PREV_PASSWORD_5 | | | VARCHAR2 | |
| NAVIGATIONHTML | NAVIGATIONHTML_ID | | | NUMBER | |
| NAVIGATIONHTML | DESCRIPTION | | | VARCHAR2 | |
| NAVIGATIONHTML | URL | | | VARCHAR2 | |
| NAVIGATIONHTML | INDENT | | | VARCHAR2 | |
| NAVIGATIONHTML | MODULE | | | VARCHAR2 | |
| NAVIGATIONHTML | SORTORDER | | | FLOAT | |
| NAVIGATIONHTML | NAVIGATIONHTML_MASTER | | | VARCHAR2 | |
| NAVIGATIONHTML | NAVIGATIONHTML_LEVEL | | | NUMBER | |
| NAVIGATIONHTML | PAGE | | | VARCHAR2 | |
| NAVIGATIONHTML | APPLICATION_FLAG | | | VARCHAR2 | |
| NON_RESULT_VALUES | NON_RESULT_ID | | | NUMBER | |
| NON_RESULT_VALUES | SERVICE_PROVIDER_ID | | | NUMBER | |
| NON_RESULT_VALUES | NON_RESULT_VALUE | | | VARCHAR2 | |
| NON_RESULT_VALUES | REJECT_IF_FOUND_FLAG | | | VARCHAR2 | |
| NON_RESULT_VALUES | NOTIFY_IF_FOUND_FLAG | | | VARCHAR2 | |
| NON_RESULT_VALUES | STATUS | | | VARCHAR2 | |
| ONTIME_ABC_CALCULATOR | SUM_PATIENT_NUM | | | NUMBER | |
| ONTIME_ABC_CALCULATOR | PATIENT_NUM | | | NUMBER | |
| ONTIME_ABC_CALCULATOR | ONTIME_PATIENT_NUM | | | NUMBER | |
| ONTIME_ABC_CALCULATOR | BAYESIAN_APF | | | NUMBER | |
| ONTIME_ABC_CALCULATOR | CONTACT_ID | | | NUMBER | |
| PARSED_RESULTS | PARSED_RESULT_ID | | | NUMBER | |
| PARSED_RESULTS | BATCH_ID | | | NUMBER | |
| PARSED_RESULTS | ORGANIZATION_ID | | | NUMBER | |
| PARSED_RESULTS | LIS_ACCESSION_NUMBER | | | VARCHAR2 | |
| PARSED_RESULTS | SERVICE_ORDER_ID | | | NUMBER | |
| PARSED_RESULTS | PATIENT_ID | | | NUMBER | |
| PARSED_RESULTS | PATIENT_FIRST_NAME | | | VARCHAR2 | |
| PARSED_RESULTS | PATIENT_LAST_NAME | | | VARCHAR2 | |
| PARSED_RESULTS | DOB | | | DATE | |
| PARSED_RESULTS | SEX | | | VARCHAR2 | |
| PARSED_RESULTS | ADDRESS1 | | | VARCHAR2 | |
| PARSED_RESULTS | ADDRESS2 | | | VARCHAR2 | |
| PARSED_RESULTS | CITY | | | VARCHAR2 | |
| PARSED_RESULTS | STATE | | | VARCHAR2 | |
| PARSED_RESULTS | ZIP | | | VARCHAR2 | |
| PARSED_RESULTS | ORDERED_TEST_CODE | | | VARCHAR2 | |
| PARSED_RESULTS | RESULTED_TEST_CODE | | | VARCHAR2 | |
| PARSED_RESULTS | NUMERIC_RESULT | | | NUMBER | |
| PARSED_RESULTS | TEXT_RESULT | | | VARCHAR2 | |
| PARSED_RESULTS | RESULT_DATE | | | DATE | |
| PARSED_RESULTS | PROVIDER_ID | | | NUMBER | |
| PARSED_RESULTS | PROVIDER_FIRST_NAME | | | VARCHAR2 | |
| PARSED_RESULTS | PROVIDER_LAST_NAME | | | VARCHAR2 | |
| PARSED_RESULTS | VALID_FLAG | | | VARCHAR2 | |
| PARSED_RESULTS | PARSE_DATE | | | DATE | |
| PARSED_RESULTS | FILED_FLAG | | | VARCHAR2 | |
| PARSED_RESULTS | PATIENT_IDENTIFIER | | | VARCHAR2 | |
| PARSED_RESULTS | MESSAGE | | | VARCHAR2 | |
| PARSED_RESULTS | CLIENT_ID | | | NUMBER | |
| PARSED_RESULTS | PROVIDER_IDENTIFIER | | | VARCHAR2 | |
| PARSED_RESULTS | LIS_COLLECT_DATE | | | DATE | |
| PARSED_RESULTS | LIS_RECEIVED_DATE | | | DATE | |
| PARSED_RESULTS | LIS_COLLECT_TIME | | | DATE | |
| PARSED_RESULTS | LIS_RECEIVED_TIME | | | DATE | |
| PARSED_RESULTS | RESULT_TIME | | | DATE | |
| PARSED_RESULTS | RESULT_RANGE | | | VARCHAR2 | |
| PATIENT_ACCOUNT | PATIENT_ID | | | NUMBER | |
| PATIENT_ACCOUNT | PATIENT_ACCOUNT_STATUS | | | NUMBER | |
| PATIENT_ACCOUNT | ACCOUNT_ID | | | NUMBER | |
| PATIENT_ACCOUNT_CHANGE_HISTORY | DATE_TIME | | | DATE | |
| PATIENT_ACCOUNT_CHANGE_HISTORY | PATIENT_ID | | | NUMBER | |
| PATIENT_ACCOUNT_CHANGE_HISTORY | ACCOUNT_ID | | | NUMBER | |
| PATIENT_ACCOUNT_CHANGE_HISTORY | LATEST_STATUS | | | VARCHAR2 | |
| PATIENT_ACCOUNT_CHANGE_HISTORY | REASON_FOR_CHANGE | | | VARCHAR2 | |
| PATIENT_ACCOUNT_STATUS_LOOKUP | PATIENT_ACCOUNT_STATUS | | | NUMBER | |
| PATIENT_ACCOUNT_STATUS_LOOKUP | STATUS_NAME | | | VARCHAR2 | |
| PATIENT_ALIAS | LAST_NAME | | | VARCHAR2 | |
| PATIENT_ALIAS | FIRST_NAME | | | VARCHAR2 | |
| PATIENT_ALIAS | MIDDLE_NAME | | | VARCHAR2 | |
| PATIENT_ALIAS | PATIENT_ID | | | NUMBER | |
| PATIENT_STATUS | PATIENT_ID | | | NUMBER | |
| PATIENT_STATUS | PATIENT_STATUS_ID | | | NUMBER | |
| PATIENT_STATUS | RESEARCH | | | NUMBER | |
| PATIENT_STATUS | QUALITY_IMPROVEMENT | | | NUMBER | |
| PAYOR_ACCOUNT | ORGANIZATION_ID | | | NUMBER | |
| PAYOR_ACCOUNT | ACCOUNT_ID | | | NUMBER | |
| PAYOR_ACCOUNT | NAME | | | VARCHAR2 | |
| PAYOR_ACCOUNT | DESCRIPTION | | | VARCHAR2 | |
| PAYOR_ACCOUNT | CONTACT_ID | | | NUMBER | |
| PETHNIC_LOOKUP | CODE | | | VARCHAR2 | |
| PETHNIC_LOOKUP | DESCRIPTION | | | VARCHAR2 | |
| PETHNIC_LOOKUP | ACTIVE | | | VARCHAR2 | |
| PETHNIC_LOOKUP | SORT_ORDER | | | NUMBER | |
| PGUAR_REL_LOOKUP | CODE | | | VARCHAR2 | |
| PGUAR_REL_LOOKUP | DESCRIPTION | | | VARCHAR2 | |
| PGUAR_REL_LOOKUP | ACTIVE | | | VARCHAR2 | |
| PGUAR_REL_LOOKUP | SORT_ORDER | | | NUMBER | |
| PHYSICIAN_SAMPLE_PERCENTAGE | LOW_PATIENT_PERCENTAGE | | | NUMBER | |
| PHYSICIAN_SAMPLE_PERCENTAGE | MEDIUM_PATIENT_PERCENTAGE | | | NUMBER | |
| PHYSICIAN_SAMPLE_PERCENTAGE | HIGH_PATIENT_PERCENTAGE | | | NUMBER | |
| PHYSICIAN_SAMPLE_PERCENTAGE | TEST_CODE | | | VARCHAR2 | |
| PHYSICIAN_SAMPLE_PERCENTAGE | PRIMARY_PROVIDER | | | NUMBER | |
| PHYS_STAT_LOOKUP | PHYS_STATUS | | | VARCHAR2 | |
| PHYS_STAT_LOOKUP | DESCRIPTION | | | VARCHAR2 | |
| PL_ACCOUNT | CLIENT_ID | | | NUMBER | |
| PL_ACCOUNT | ACCOUNT_NUMBER | | | VARCHAR2 | |
| PL_ACCOUNT | ACCOUNT_NAME | | | VARCHAR2 | |
| PL_ACCOUNT | CONTRACT_STARTDATE | | | DATE | |
| PL_ACCOUNT | CONTRACT_ENDDATE | | | DATE | |
| PL_ACCOUNT | DEFAULT_DISCOUNT | | | FLOAT | |
| PL_ACCOUNT | FEE_SCHEDULE_ID | | | NUMBER | |
| PL_ACCOUNT | RANK | | | NUMBER | |
| PL_ACCOUNT | CHECK_COMPLIANCE | | | VARCHAR2 | |
| PL_ACCOUNT | SERVICE_PROVIDER_ID | | | NUMBER | |
| PL_CONTAINER | CONTAINER_TYPE | | | VARCHAR2 | |
| PL_CONTAINER | MAX_VOLUME | | | FLOAT | |
| PL_CONTAINER | PRESERVATIVE_TYPE | | | VARCHAR2 | |
| PL_CONTAINER | UNIT_TYPE | | | VARCHAR2 | |
| PL_CONTAINER | SHORTNAME | | | VARCHAR2 | |
| PL_GROUP | GROUP_NAME | | | VARCHAR2 | |
| PL_GROUP | GROUP_DESCRIPTION | | | VARCHAR2 | |
| PL_GROUP | GROUP_TYPE | | | VARCHAR2 | |
| PL_GROUP | APPLICATION_FLAG | | | VARCHAR2 | |
| PL_LOCATION | LOCATION_ID | | | NUMBER | |
| PL_LOCATION | LOCATION_NAME | | | VARCHAR2 | |
| PL_LOCATION | CODE | | | VARCHAR2 | |
| PL_LOCATION | ADDRESS_1 | | | VARCHAR2 | |
| PL_LOCATION | ADDRESS_2 | | | VARCHAR2 | |
| PL_LOCATION | CITY | | | VARCHAR2 | |
| PL_LOCATION | STATE | | | VARCHAR2 | |
| PL_LOCATION | ZIP | | | VARCHAR2 | |
| PL_LOCATION | COUNTRY | | | VARCHAR2 | |
| PL_LOCATION | PHONE | | | VARCHAR2 | |
| PL_LOCATION | PHONE_2 | | | VARCHAR2 | |
| PL_LOCATION | FAX | | | VARCHAR2 | |
| PL_LOCATION | HOURS | | | VARCHAR2 | |
| PL_LOCATION | SUPPLY_DIST | | | VARCHAR2 | |
| PL_LOCATION_CHANGE_HISTORY | CREATE_TIMESTAMP | | | DATE | |
| PL_LOCATION_CHANGE_HISTORY | LOCATION_ID | | | NUMBER | |
| PL_LOCATION_CHANGE_HISTORY | LOCATION_NAME | | | VARCHAR2 | |
| PL_LOCATION_CHANGE_HISTORY | CODE | | | VARCHAR2 | |
| PL_LOCATION_CHANGE_HISTORY | ADDRESS_1 | | | VARCHAR2 | |
| PL_LOCATION_CHANGE_HISTORY | ADDRESS_2 | | | VARCHAR2 | |
| PL_LOCATION_CHANGE_HISTORY | CITY | | | VARCHAR2 | |
| PL_LOCATION_CHANGE_HISTORY | STATE | | | VARCHAR2 | |
| PL_LOCATION_CHANGE_HISTORY | ZIP | | | VARCHAR2 | |
| PL_LOCATION_CHANGE_HISTORY | COUNTRY | | | VARCHAR2 | |
| PL_LOCATION_CHANGE_HISTORY | PHONE | | | VARCHAR2 | |
| PL_LOCATION_CHANGE_HISTORY | PHONE_2 | | | VARCHAR2 | |
| PL_LOCATION_CHANGE_HISTORY | FAX | | | VARCHAR2 | |
| PL_LOCATION_CHANGE_HISTORY | HOURS | | | VARCHAR2 | |
| PL_LOCATION_CHANGE_HISTORY | SUPPLY_DIST | | | VARCHAR2 | |
| PL_LOCATION_CHANGE_HISTORY | REASON_FOR_CHANGE | | | VARCHAR2 | |
| PL_LOCATION_CHANGE_HISTORY | LATEST_STATUS | | | VARCHAR2 | |
| PL_ORGANIZATION | ORGANIZATION_ID | | | NUMBER | |
| PL_ORGANIZATION | ORGANIZATION_NAME | | | VARCHAR2 | |
| PL_ORGANIZATION | LOCATION_ID | | | NUMBER | |
| PL_ORGANIZATION | IS_CLIENT | | | VARCHAR2 | |
| PL_ORGANIZATION | IS_SERVICE_PROVIDER | | | VARCHAR2 | |
| PL_ORGANIZATION | IS_PARENT | | | VARCHAR2 | |
| PL_ORGANIZATION | IS_NETWORK | | | VARCHAR2 | |
| PL_PROCEDURE | PROC_CODE | | | VARCHAR2 | |
| PL_PROCEDURE | PROCEDURE_NAME | | | VARCHAR2 | |
| PL_PROCEDURE | SHORT_NAME | | | VARCHAR2 | |
| PL_PROCEDURE | MODALITY | | | VARCHAR2 | |
| PL_PROCEDURE | ALPHA_NAME | | | VARCHAR2 | |
| PL_PROCEDURE | PROC_NOTE | | | VARCHAR2 | |
| PL_PROCEDURE | CPT_CODE | | | VARCHAR2 | |
| PL_PROCEDURE | PROC_VERSION | | | NUMBER | |
| PL_PROCEDURE | PROFILE_VERSION | | | NUMBER | |
| PL_PROCEDURE | TEST_VERSION | | | NUMBER | |
| PL_PROCEDURE | PROC_EFFECTIVE_DATE | | | DATE | |
| PL_PROCEDURE | PROC_ACTIVE_STATUS | | | VARCHAR2 | |
| PL_ROLE | ROLE_ID | | | VARCHAR2 | |
| PL_ROLE | ROLE_DESCRIPTION | | | VARCHAR2 | |
| PROVIDER_ACCOUNT | SERVICE_PROVIDER_ID | | | NUMBER | |
| PROVIDER_ACCOUNT | ACCOUNT_NUMBER | | | VARCHAR2 | |
| PSEX_LOOKUP | CODE | | | VARCHAR2 | |
| PSEX_LOOKUP | DESCRIPTION | | | VARCHAR2 | |
| PSEX_LOOKUP | ACTIVE | | | VARCHAR2 | |
| PSEX_LOOKUP | SORT_ORDER | | | NUMBER | |
| PSTATUS_LOOKUP | CODE | | | VARCHAR2 | |
| PSTATUS_LOOKUP | DESCRIPTION | | | VARCHAR2 | |
| PSTATUS_LOOKUP | ACTIVE | | | VARCHAR2 | |
| PSTATUS_LOOKUP | SORT_ORDER | | | NUMBER | |
| REFERENCE_RANGE | SERVICE_PROVIDER_ID | | | NUMBER | |
| REFERENCE_RANGE | SERVICE_CODE | | | VARCHAR2 | |
| REFERENCE_RANGE | RANGE_ID | | | NUMBER | |
| REFERENCE_RANGE | EFFECTIVE_DATE | | | DATE | |
| REFERENCE_RANGE | INACTIVE_DATE | | | DATE | |
| REFERENCE_RANGE | RANGE_STATUS | | | VARCHAR2 | |
| REFERENCE_RANGE | AGE | | | NUMBER | |
| REFERENCE_RANGE | SEX | | | VARCHAR2 | |
| REFERENCE_RANGE | PHYS_STATUS | | | VARCHAR2 | |
| REFERENCE_RANGE | UNIT | | | VARCHAR2 | |
| REFERENCE_RANGE | CRITICAL_LOW | | | VARCHAR2 | |
| REFERENCE_RANGE | CRITICAL_HIGH | | | VARCHAR2 | |
| REFERENCE_RANGE | NORMAL_LOW | | | VARCHAR2 | |
| REFERENCE_RANGE | NORMAL_HIGH | | | VARCHAR2 | |
| REFERENCE_RANGE | TEXTUAL_NORMAL | | | VARCHAR2 | |
| REFERENCE_RANGE | DELTA | | | VARCHAR2 | |
| REFERENCE_RANGE | SORT_ORDER | | | NUMBER | |
| REFERENCE_UNITS | UNIT | | | VARCHAR2 | |
| REFERENCE_UNITS | DESCRIPTION | | | VARCHAR2 | |
| REMINDER_TRUTH_TABLE | TEST_TYPE | | | VARCHAR2 | |
| REMINDER_TRUTH_TABLE | REMINDER_TYPE | | | VARCHAR2 | |
| REMINDER_TRUTH_TABLE | RESULT_RANGE | | | VARCHAR2 | |
| REMINDER_TRUTH_TABLE | CANNED_TEXT_IDS | | | NUMBER | |
| REMINDER_TRUTH_TABLE_TMP | TEST_TYPE | | | VARCHAR2 | |
| REMINDER_TRUTH_TABLE_TMP | REMINDER_TYPE | | | VARCHAR2 | |
| REMINDER_TRUTH_TABLE_TMP | RESULT_RANGE | | | VARCHAR2 | |
| REMINDER_TRUTH_TABLE_TMP | SEQUENCE_NO | | | NUMBER | |
| REMINDER_TRUTH_TABLE_TMP | CANNED_TEXT_IDS | | | NUMBER | |
| REPORT | REPORT_NUMBER | | | VARCHAR2 | |
| REPORT | REPORT_NAME | | | VARCHAR2 | |
| REPORT | REPORT_TYPE | | | VARCHAR2 | |
| REPORT | ORGANIZATION_ID | | | NUMBER | |
| REPORT_DATA | REPORT_DATA_ID | | | NUMBER | |
| REPORT_DATA | OUTPUT_TYPE_ID | | | NUMBER | |
| REPORT_DATA | PRACTICE_ID | | | NUMBER | |
| REPORT_DATA | FILE_NAME | | | VARCHAR2 | |
| REPORT_DATA | CONTACT_ID | | | NUMBER | |
| REPORT_DATA | REPORT_STATUS | | | VARCHAR2 | |
| REPORT_DATA | OUTPUT_DATE | | | DATE | |
| RESULT_ABC_CALCULATOR | SUM_PATIENT_NUM | | | NUMBER | |
| RESULT_ABC_CALCULATOR | PATIENT_NUM | | | NUMBER | |
| RESULT_ABC_CALCULATOR | LOW_PATIENT_NUM | | | NUMBER | |
| RESULT_ABC_CALCULATOR | MEDIUM_PATIENT_NUM | | | NUMBER | |
| RESULT_ABC_CALCULATOR | HIGH_PATIENT_NUM | | | NUMBER | |
| RESULT_ABC_CALCULATOR | BAYESIAN_APF | | | NUMBER | |
| RESULT_ABC_CALCULATOR | CONTACT_ID | | | NUMBER | |
| RESULT_ABC_CALCULATOR | TEST_ID | | | VARCHAR2 | |
| RESULT_ABC_CALCULATOR_2 | SUM_PATIENT_NUM | | | NUMBER | |
| RESULT_ABC_CALCULATOR_2 | PATIENT_NUM | | | NUMBER | |
| RESULT_ABC_CALCULATOR_2 | LOW_PATIENT_NUM | | | NUMBER | |
| RESULT_ABC_CALCULATOR_2 | MEDIUM_PATIENT_NUM | | | NUMBER | |
| RESULT_ABC_CALCULATOR_2 | HIGH_PATIENT_NUM | | | NUMBER | |
| RESULT_ABC_CALCULATOR_2 | BAYESIAN_APF | | | NUMBER | |
| RESULT_ABC_CALCULATOR_2 | CONTACT_ID | | | NUMBER | |
| RESULT_ABC_CALCULATOR_2 | TEST_ID | | | VARCHAR2 | |
| RESULT_ABC_STEP | PATIENT_ID | | | NUMBER | |
| RESULT_ABC_STEP | RANGE_TYPE | | | VARCHAR2 | |
| RESULT_ABC_STEP | CONTACT_ID | | | NUMBER | |
| RESULT_ABC_STEP | TEST_ID | | | VARCHAR2 | |
| RESULT_LOOKUP | CODE | | | VARCHAR2 | |
| RESULT_LOOKUP | DESCRIPTION | | | VARCHAR2 | |
| RESULT_LOOKUP | RESULT_TYPE | | | VARCHAR2 | |
| RESULT_LOOKUP | ACTIVE | | | VARCHAR2 | |
| RESULT_LOOKUP | SORT_ORDER | | | NUMBER | |
| RESULT_LOOKUP | PRIORITY | | | NUMBER | |
| RESULTS_SOURCE | ROW_KEY | | | NUMBER | |
| RESULTS_SOURCE | MRN | | | CHAR | |
| RESULTS_SOURCE | LNAME | | | CHAR | |
| RESULTS_SOURCE | FNAME | | | CHAR | |
| RESULTS_SOURCE | MI | | | CHAR | |
| RESULTS_SOURCE | DOB | | | CHAR | |
| RESULTS_SOURCE | SEX | | | CHAR | |
| RESULTS_SOURCE | LIS_COLLECT_DATE | | | CHAR | |
| RESULTS_SOURCE | LIS_ACCESSION_NUM | | | CHAR | |
| RESULTS_SOURCE | SERVICE_CODE | | | CHAR | |
| RESULTS_SOURCE | PARENT_CODE | | | CHAR | |
| RESULTS_SOURCE | ORDERING_PROV_ID | | | CHAR | |
| RESULTS_SOURCE | ORDERING_CLIENT | | | CHAR | |
| RESULTS_SOURCE | PERFORMED_AT | | | CHAR | |
| RESULTS_SOURCE | UNIT | | | CHAR | |
| RESULTS_SOURCE | TEST_RESULT_DETAIL_NUMERIC | | | CHAR | |
| RESULTS_SOURCE | TEXT | | | CHAR | |
| RESULTS_SOURCE | LIS_RECEIVED_DATE | | | CHAR | |
| RESULTS_SOURCE | RESULT_DATE | | | CHAR | |
| SAMPLE_PERCENTAGE_MLDL | PATIENT_ID | | | NUMBER | |
| SAMPLE_PERCENTAGE_MLDL | LOW_PATIENTS | | | CHAR | |
| SAMPLE_PERCENTAGE_MLDL | MEDIUM_PATIENTS | | | CHAR | |
| SAMPLE_PERCENTAGE_MLDL | HIGH_PATIENTS | | | CHAR | |
| SAMPLE_PERCENTAGE_MLDL | CONTACT_ID | | | NUMBER | |
| SERVICE_DIAGNOSIS | SERVICE_ORDER_ID | | | NUMBER | |
| SERVICE_DIAGNOSIS | ICD9_CODE | | | VARCHAR2 | |
| SERVICE_RULES | SERVICE_CODE | | | VARCHAR2 | |
| SERVICE_RULES | SERVICE_RULE | | | VARCHAR2 | |
| SERVICE_RULES | SERVICE_RULE_TYPE | | | VARCHAR2 | |
| SITE_METADATA | METADATA_ID | | | NUMBER | |
| SITE_METADATA | ORGANIZATION_ID | | | NUMBER | |
| SITE_METADATA | METADATA_VALUE | | | VARCHAR2 | |
| SOFTWARE_RELEASE | RELEASE_ID | | | VARCHAR2 | |
| SOFTWARE_RELEASE | SOFTWARE_RELEASE_DATE | | | DATE | |
| SOFTWARE_RELEASE | RELEASE_NOTES | | | LONG | |
| TEST_COLLECTION | SERVICE_PROVIDER_ID | | | NUMBER | |
| TEST_COLLECTION | SERVICE_CODE | | | VARCHAR2 | |
| TEST_COLLECTION | COLLECTION_GROUP_ID | | | NUMBER | |
| TEST_COLLECTION | SOURCE_TYPE | | | VARCHAR2 | |
| TEST_COLLECTION | CONTAINER_TYPE | | | VARCHAR2 | |
| TEST_COLLECTION | RELATION | | | VARCHAR2 | |
| TEST_COLLECTION | PREFERRED | | | VARCHAR2 | |
| TEST_COLLECTION | CONSOLIDATION_FLAG | | | VARCHAR2 | |
| TEST_COLLECTION | CONTAINER_QTY | | | NUMBER | |
| TEST_COLLECTION | VOLUME | | | FLOAT | |
| TEST_COLLECTION | MIN_VOLUME | | | FLOAT | |
| TEST_COLLECTION | UNIT_TYPE | | | VARCHAR2 | |
| TEST_COLLECTION | COLLECT_NOTE | | | VARCHAR2 | |
| TEST_COLLECTION | EFFECTIVE_DATE | | | DATE | |
| TEST_COLLECTION | CLIENT_STORAGE | | | VARCHAR2 | |
| TEST_COLLECTION | LAB_STORAGE | | | VARCHAR2 | |
| TEST_COLLECTION | PROCESSING_REQUIRED | | | VARCHAR2 | |
| TEST_COLLECTION | TEST_COLLECTION_VERSION | | | NUMBER | |
| TEST_LOOKUP | TEST_ID | | | VARCHAR2 | |
| TEST_LOOKUP | TEST_NAME | | | VARCHAR2 | |
| TEST_ORDER_INFO | SERVICE_ORDER_ID | | | NUMBER | |
| TEST_ORDER_INFO | SERVICE_PROVIDER_ID | | | NUMBER | |
| TEST_ORDER_INFO | SERVICE_CODE | | | VARCHAR2 | |
| TEST_ORDER_INFO | COLLECT_DATE | | | DATE | |
| TEST_ORDER_INFO | SOURCE_TYPE | | | VARCHAR2 | |
| TEST_ORDER_INFO | COLLECT_TEXT | | | VARCHAR2 | |
| TEST_ORDER_PROFILE | SERVICE_ORDER_ID | | | NUMBER | |
| TEST_ORDER_PROFILE | ORDERED_SERVICE_PROVIDER_ID | | | NUMBER | |
| TEST_ORDER_PROFILE | ORDERED_SERVICE_CODE | | | VARCHAR2 | |
| TEST_ORDER_PROFILE | PARENT_SERVICE_PROVIDER_ID | | | NUMBER | |
| TEST_ORDER_PROFILE | PARENT_SERVICE_CODE | | | VARCHAR2 | |
| TEST_ORDER_PROFILE | CHILD_SERVICE_PROVIDER_ID | | | NUMBER | |
| TEST_ORDER_PROFILE | CHILD_SERVICE_CODE | | | VARCHAR2 | |
| TEST_ORDER_PROFILE | PARENT_SORT_ORDER | | | NUMBER | |
| TEST_ORDER_PROFILE | CHILD_SORT_ORDER | | | NUMBER | |
| TEST_ORDER_PROFILE | PARENT_VERSION | | | NUMBER | |
| TEST_ORDER_PROFILE | CHILD_VERSION | | | NUMBER | |
| TEST_ORDER_PROFILE | RESULTABLE | | | VARCHAR2 | |
| TEST_ORDER_PROFILE | PERFORM_ORDERED_SERV_PROV_ID | | | NUMBER | |
| TEST_ORDER_PROFILE | PERFORM_ORDERED_SERVICE_CODE | | | VARCHAR2 | |
| TEST_ORDER_PROFILE | PERFORM_PARENT_SERV_PROV_ID | | | NUMBER | |
| TEST_ORDER_PROFILE | PERFORM_PARENT_SERVICE_CODE | | | VARCHAR2 | |
| TEST_ORDER_PROFILE | PERFORM_CHILD_SERV_PROV_ID | | | NUMBER | |
| TEST_ORDER_PROFILE | PERFORM_CHILD_SERVICE_CODE | | | VARCHAR2 | |
| TEST_RESULT_INTERP | LAB_ID | | | NUMBER | |
| TEST_RESULT_INTERP | TEST_CODE | | | VARCHAR2 | |
| TEST_RESULT_INTERP | TEXT | | | VARCHAR2 | |
| TEST_RESULT_INTERP | INTERP_TEXT | | | VARCHAR2 | |
| TEST_RESULT_INTERP | DISABLE_REMINDER | | | VARCHAR2 | |
| TRANSLATION_TABLE | TEST_TYPE | | | VARCHAR2 | |
| TRANSLATION_TABLE | REMINDER_TYPE | | | VARCHAR2 | |
| TRANSLATION_TABLE | CANNED_TEXT_IDS | | | NUMBER | |
| UNIT_TYPE | UNIT_TYPE | | | VARCHAR2 | |
| UNIT_TYPE | UNIT_TYPE_DESCRIPTION | | | VARCHAR2 | |
| UNIT_TYPE | BASE_UNIT_CONVERSION | | | FLOAT | |
| UNIT_TYPE | BASE_UNIT_TYPE | | | VARCHAR2 | |
| V_LAB | SERVICE_PROVIDER_ID | | | NUMBER | |
| V_LAB | SERVICE_PROVIDER_NAME | | | VARCHAR2 | |
| V_LAB | LOCATION_ID | | | NUMBER | |
| V_LAB | ADDRESS_1 | | | VARCHAR2 | |
| V_LAB | ADDRESS_2 | | | VARCHAR2 | |
| V_LAB | CITY | | | VARCHAR2 | |
| V_LAB | STATE | | | VARCHAR2 | |
| V_LAB | COUNTRY | | | VARCHAR2 | |
| V_LAB | ZIP | | | VARCHAR2 | |
| V_LAB | PHONE | | | VARCHAR2 | |
| V_LAB | PHONE_2 | | | VARCHAR2 | |
| V_LAB | FAX | | | VARCHAR2 | |
| V_LAB | HOURS | | | VARCHAR2 | |
| V_ORDERED_PARENT_RESULTED | ORDERED | | | VARCHAR2 | |
| V_ORDERED_PARENT_RESULTED | PARENT | | | VARCHAR2 | |
| V_ORDERED_PARENT_RESULTED | RESULTED | | | VARCHAR2 | |
| V_ORDERED_PARENT_RESULTED | SERVICE_CODE | | | VARCHAR2 | |
| V_ORDERED_PARENT_RESULTED | TEST_CODE | | | VARCHAR2 | |
| V_ORDERED_PARENT_RESULTED | SERVICE_PROVIDER_ID | | | NUMBER | |
| V_ORDERED_PARENT_RESULTED | SORT_ORDER | | | NUMBER | |
| V_PATIENT | PATIENT_ID | | | NUMBER | |
| V_PATIENT | LAST_NAME | | | VARCHAR2 | |
| V_PATIENT | FIRST_NAME | | | VARCHAR2 | |
| V_PATIENT | MIDDLE_NAME | | | VARCHAR2 | |
| V_PATIENT | DOB | | | DATE | |
| V_PATIENT | PREFIX | | | VARCHAR2 | |
| V_PATIENT | SUFFIX | | | VARCHAR2 | |
| V_PATIENT | SPOUSE | | | VARCHAR2 | |
| V_PATIENT | LAST_MESSAGE_ID | | | NUMBER | |
| V_PATIENT | GUARANTOR | | | VARCHAR2 | |
| V_PATIENT | EMPLOYER_SCHOOL | | | VARCHAR2 | |
| V_PATIENT | PATIENT_HOME_LOCATION | | | NUMBER | |
| V_PATIENT | CURRENT_PROVIDER | | | NUMBER | |
| V_PATIENT | PRIMARY_PROVIDER | | | NUMBER | |
| V_PATIENT | DATE_OF_DEATH | | | DATE | |
| V_PATIENT | SEX | | | VARCHAR2 | |
| V_PATIENT | GUAR_RELATION | | | VARCHAR2 | |
| V_PATIENT | MARITAL_STATUS | | | VARCHAR2 | |
| V_PATIENT | ETHNIC_GROUP | | | VARCHAR2 | |
| V_PATIENT | SPECIES | | | VARCHAR2 | |
| V_PATIENT | STATUS | | | VARCHAR2 | |
| V_PATIENT | DATE_TIME | | | DATE | |
| V_PATIENT | PATIENT_STATUS_ID | | | NUMBER | |
| V_PATIENT_1 | STATUS | | | VARCHAR2 | |
| V_PATIENT_1 | DATE_TIME | | | DATE | |
| V_PATIENT_1 | PATIENT_STATUS_ID | | | NUMBER | |
| V_PATIENT_1 | PATIENT_ID | | | NUMBER | |
| V_PATIENT_1 | LAST_NAME | | | VARCHAR2 | |
| V_PATIENT_1 | FIRST_NAME | | | VARCHAR2 | |
| V_PATIENT_1 | MIDDLE_NAME | | | VARCHAR2 | |
| V_PATIENT_1 | DOB | | | DATE | |
| V_PATIENT_1 | PREFIX | | | VARCHAR2 | |
| V_PATIENT_1 | SUFFIX | | | VARCHAR2 | |
| V_PATIENT_1 | SPOUSE | | | VARCHAR2 | |
| V_PATIENT_1 | LAST_MESSAGE_ID | | | NUMBER | |
| V_PATIENT_1 | GUARANTOR | | | VARCHAR2 | |
| V_PATIENT_1 | EMPLOYER_SCHOOL | | | VARCHAR2 | |
| V_PATIENT_1 | PATIENT_HOME_LOCATION | | | NUMBER | |
| V_PATIENT_1 | CURRENT_PROVIDER | | | NUMBER | |
| V_PATIENT_1 | PRIMARY_PROVIDER | | | NUMBER | |
| V_PATIENT_1 | DATE_OF_DEATH | | | DATE | |
| V_PATIENT_1 | SEX | | | VARCHAR2 | |
| V_PATIENT_1 | GUAR_RELATION | | | VARCHAR2 | |
| V_PATIENT_1 | MARITAL_STATUS | | | VARCHAR2 | |
| V_PATIENT_1 | ETHNIC_GROUP | | | VARCHAR2 | |
| V_PATIENT_1 | SPECIES | | | VARCHAR2 | |
| V_PATIENT_2 | PATIENT_ID | | | NUMBER | |
| V_PATIENT_2 | LAST_NAME | | | VARCHAR2 | |
| V_PATIENT_2 | FIRST_NAME | | | VARCHAR2 | |
| V_PATIENT_2 | MIDDLE_NAME | | | VARCHAR2 | |
| V_PATIENT_2 | DOB | | | DATE | |
| V_PATIENT_2 | PREFIX | | | VARCHAR2 | |
| V_PATIENT_2 | SUFFIX | | | VARCHAR2 | |
| V_PATIENT_2 | SPOUSE | | | VARCHAR2 | |
| V_PATIENT_2 | LAST_MESSAGE_ID | | | NUMBER | |
| V_PATIENT_2 | GUARANTOR | | | VARCHAR2 | |
| V_PATIENT_2 | EMPLOYER_SCHOOL | | | VARCHAR2 | |
| V_PATIENT_2 | PATIENT_HOME_LOCATION | | | NUMBER | |
| V_PATIENT_2 | CURRENT_PROVIDER | | | NUMBER | |
| V_PATIENT_2 | PRIMARY_PROVIDER | | | NUMBER | |
| V_PATIENT_2 | DATE_OF_DEATH | | | DATE | |
| V_PATIENT_2 | SEX | | | VARCHAR2 | |
| V_PATIENT_2 | GUAR_RELATION | | | VARCHAR2 | |
| V_PATIENT_2 | MARITAL_STATUS | | | VARCHAR2 | |
| V_PATIENT_2 | ETHNIC_GROUP | | | VARCHAR2 | |
| V_PATIENT_2 | SPECIES | | | VARCHAR2 | |
| V_PATIENT_2 | STATUS | | | VARCHAR2 | |
| V_PATIENT_2 | PATIENT_STATUS_ID | | | NUMBER | |
| V_PATIENT_STATUS | | | | | |
| V_PATIENT_STATUS | PATIENT_ID | | | NUMBER | |
| V_PATIENT_STATUS | LAST_NAME | | | VARCHAR2 | |
| V_PATIENT_STATUS | FIRST_NAME | | | VARCHAR2 | |
| V_PATIENT_STATUS | MIDDLE_NAME | | | VARCHAR2 | |
| V_PATIENT_STATUS | DOB | | | DATE | |
| V_PATIENT_STATUS | PREFIX | | | VARCHAR2 | |
| V_PATIENT_STATUS | SUFFIX | | | VARCHAR2 | |
| V_PATIENT_STATUS | SPOUSE | | | VARCHAR2 | |
| V_PATIENT_STATUS | LAST_MESSAGE_ID | | | NUMBER | |
| V_PATIENT_STATUS | GUARANTOR | | | VARCHAR2 | |
| V_PATIENT_STATUS | EMPLOYER_SCHOOL | | | VARCHAR2 | |
| V_PATIENT_STATUS | PATIENT_HOME_LOCATION | | | NUMBER | |
| V_PATIENT_STATUS | CURRENT_PROVIDER | | | NUMBER | |
| V_PATIENT_STATUS | PRIMARY_PROVIDER | | | NUMBER | |
| V_PATIENT_STATUS | DATE_OF_DEATH | | | DATE | |
| V_PATIENT_STATUS | SEX | | | VARCHAR2 | |
| V_PATIENT_STATUS | GUAR_RELATION | | | VARCHAR2 | |
| V_PATIENT_STATUS | MARITAL_STATUS | | | VARCHAR2 | |
| V_PATIENT_STATUS | ETHNIC_GROUP | | | VARCHAR2 | |
| V_PATIENT_STATUS | SPECIES | | | VARCHAR2 | |
| V_PATIENT_STATUS | STATUS | | | VARCHAR2 | |
| V_PATIENT_STATUS | PATIENT_STATUS_ID | | | NUMBER | |
| V_PHYSICIAN | CONTACT_ID | | | NUMBER | |
| V_PHYSICIAN | LAST_NAME | | | VARCHAR2 | |
| V_PHYSICIAN | FIRST_NAME | | | VARCHAR2 | |
| V_PHYSICIAN | MIDDLE_NAME | | | VARCHAR2 | |
| V_PHYSICIAN | PREFIX | | | VARCHAR2 | |
| V_PHYSICIAN | SUFFIX | | | VARCHAR2 | |
| V_PHYSICIAN | TITLE | | | VARCHAR2 | |
| V_PHYSICIAN | WORK_PHONE | | | VARCHAR2 | |
| V_PHYSICIAN | HOME_PHONE | | | VARCHAR2 | |
| V_PHYSICIAN | MOBILE_PHONE | | | VARCHAR2 | |
| V_PHYSICIAN | FAX | | | VARCHAR2 | |
| V_PHYSICIAN | EMAIL | | | VARCHAR2 | |
| V_PHYSICIAN | USERNAME | | | VARCHAR2 | |
| V_PHYSICIAN | PIN | | | VARCHAR2 | |
| V_PHYSICIAN | CLIENT_ID | | | NUMBER | |
| V_PHYSICIAN | EMPLOYMENT_DATE | | | DATE | |
| V_PHYSICIAN | TERMINATION_DATE | | | DATE | |
| V_PHYSICIAN | STATUS | | | VARCHAR2 | |
| V_PHYSICIAN | LOCATION_NAME | | | VARCHAR2 | |
| V_PHYSICIAN | ADDRESS_1 | | | VARCHAR2 | |
| V_PHYSICIAN | ADDRESS_2 | | | VARCHAR2 | |
| V_PHYSICIAN | CITY | | | VARCHAR2 | |
| V_PHYSICIAN | STATE | | | VARCHAR2 | |
| V_PHYSICIAN | ZIP | | | VARCHAR2 | |
| V_PHYSICIAN | COUNTRY | | | VARCHAR2 | |
| V_PHYSICIAN | LOC_PHONE | | | VARCHAR2 | |
| V_PHYSICIAN | LOC_FAX | | | VARCHAR2 | |
| V_PKG_BAT_TEST | PKG | | | VARCHAR2 | |
| V_PKG_BAT_TEST | BATTERY | | | VARCHAR2 | |
| V_PKG_BAT_TEST | TEST | | | VARCHAR2 | |
| V_PKG_BAT_TEST | SERVICE_CODE | | | VARCHAR2 | |
| V_PKG_BAT_TEST | TEST_CODE | | | VARCHAR2 | |
| V_PKG_BAT_TEST | SERVICE_PROVIDER_ID | | | NUMBER | |
| V_PRACTICE | CLIENT_ID | | | NUMBER | |
| V_PRACTICE | CLIENT_NAME | | | VARCHAR2 | |
| V_PRACTICE | ADDED_DATE | | | VARCHAR2 | |
| V_PRACTICE | CONTACT_ID | | | NUMBER | |
| V_PRACTICE | STATUS | | | VARCHAR2 | |
| V_PRACTICE | PHYSICIAN_REFRACTORY_PERIOD | | | NUMBER | |
| V_PRACTICE | PATIENT_REFRACTORY_PERIOD | | | NUMBER | |
| V_PRACTICE | FAX_START_TIME | | | DATE | |
| V_PRACTICE | FAX_STOP_TIME | | | DATE | |
| V_PROVIDER_CHANGE_HISTORY | DATE_TIME | | | DATE | |
| V_PROVIDER_CHANGE_HISTORY | PATIENT_ID | | | NUMBER | |
| V_PROVIDER_CHANGE_HISTORY | NEW_PRIMARY_PROVIDER_ID | | | NUMBER | |
| V_PROVIDER_CHANGE_HISTORY | NEW_CONTACT_PREFIX | | | VARCHAR2 | |
| V_PROVIDER_CHANGE_HISTORY | NEW_CONTACT_NAME | | | VARCHAR2 | |
| V_PROVIDER_CHANGE_HISTORY | PREVIOUS_CONTACT_PREFIX | | | NUMBER | |
| V_PROVIDER_CHANGE_HISTORY | PREVIOUS_PRIMARY_PROVIDER_ID | | | VARCHAR2 | |
| V_PROVIDER_CHANGE_HISTORY | PREVIOUS_CONTACT_NAME | | | VARCHAR2 | |
| V_PROVIDER_CHANGE_HISTORY | LATEST_STATUS | | | VARCHAR2 | |
| V_PROVIDER_CHANGE_HISTORY | REASON_FOR_CHANGE | | | VARCHAR2 | |
| V_REPORT_LOG | REPORT_ID | | | NUMBER | |
| V_REPORT_LOG | PATIENT_ID | | | NUMBER | |
| V_REPORT_LOG | CONTACT_ID | | | NUMBER | |
| V_REPORT_LOG | CLIENT_ID | | | NUMBER | |
| V_REPORT_LOG | SEND_TIME | | | DATE | |
| V_REPORT_LOG | OUTPUT_TYPE | | | VARCHAR2 | |
| V_REPORT_LOG | REPORT_STATUS | | | VARCHAR2 | |
| V_REPORT_LOG | REPORT_FILE | | | BLOB | |
| V_REPORT_LOG | REPORT_FILE_NAME | | | VARCHAR2 | |
| V_REPORT_LOG | CANNED_TEXT_COMBINATION | | | VARCHAR2 | |
| V_REPORT_LOG | FAX_JOB_ID | | | NUMBER | |
| V_TEST_RESULT | UNIT | | | VARCHAR2 | |
| V_TEST_RESULT | INTERP_CODE | | | VARCHAR2 | |
| V_TEST_RESULT | NOTE | | | VARCHAR2 | |
| V_TEST_RESULT | STATUS | | | VARCHAR2 | |
| V_TEST_RESULT | TEST_ID | | | VARCHAR2 | |
| V_TEST_RESULT | FLOWSHEET_SENT | | | DATE | |
| V_TEST_RESULT | PATIENT_ALERT_SENT | | | DATE | |
| V_TEST_RESULT | RESULT_RANGE | | | VARCHAR2 | |
| V_TEST_RESULT | SERVICE_ORDER_ID | | | NUMBER | |
| V_TEST_RESULT | RESULT_ID | | | NUMBER | |
| V_TEST_RESULT | SERVICE_PROVIDER | | | NUMBER | |
| V_TEST_RESULT | SERVICE_CODE | | | VARCHAR2 | |
| V_TEST_RESULT | PARENT_CODE | | | VARCHAR2 | |
| V_TEST_RESULT | ORDERING_PROV_ID | | | NUMBER | |
| V_TEST_RESULT | PATIENT_ID | | | NUMBER | |
| V_TEST_RESULT | RESULT_DATE | | | DATE | |
| V_TEST_RESULT | REFERENCE_RANGE | | | VARCHAR2 | |
| VDIS_MONITOR_EMAIL_RCPT | ALERT_TYPE | | | NUMBER | |
| VDIS_MONITOR_EMAIL_RCPT | EMAIL_ADDRESS | | | VARCHAR2 | |
| VDIS_MONITOR_EMAIL_RCPT | DESCRIPTION | | | VARCHAR2 | |
| VERMONT_SAMPLE | PATIENT_ID | | | NUMBER | |
| VERMONT_SAMPLE | DT | | | DATE | |
| VERMONT_SAMPLE | PI | | | NUMBER | |
| VERMONT_SAMPLE_PERCENTAGE | LOW_PATIENT_PERCENTAGE | | | VARCHAR2 | |
| VERMONT_SAMPLE_PERCENTAGE | MEDIUM_PATIENT_PERCENTAGE | | | VARCHAR2 | |
| VERMONT_SAMPLE_PERCENTAGE | HIGH_PATIENT_PERCENTAGE | | | VARCHAR2 | |
| VERMONT_SAMPLE_PERCENTAGE | TEST_CODE | | | VARCHAR2 | |
| VM | VM1 | | | NUMBER | |
| VM | VM2 | | | NUMBER | |
| VM | VM3 | | | NUMBER | |
| VM | VM4 | | | NUMBER | |
| VM_HL7_MESSAGE_STRUCTURE | STRUCTURE_ID | | | NUMBER | |
| VM_HL7_MESSAGE_STRUCTURE | MESSAGE_NAME | | | VARCHAR2 | |
| VM_HL7_MESSAGE_STRUCTURE | SEGMENT_ID | | | VARCHAR2 | |
| VM_HL7_MESSAGE_STRUCTURE | FIELD_NAME | | | VARCHAR2 | |
| VM_HL7_MESSAGE_STRUCTURE | SEGMENT_ORDER | | | NUMBER | |
| VM_HL7_MESSAGE_STRUCTURE | FIELD_ORDER | | | NUMBER | |
| VM_HL7_MESSAGE_STRUCTURE | COMPONENT_ORDER | | | NUMBER | |
| VM_HL7_MESSAGE_STRUCTURE | SEGMENT_COUNT | | | NUMBER | |
| VM_HL7_MESSAGE_STRUCTURE | REPEAT_COUNT | | | NUMBER | |
| VM_HL7_MESSAGE_STRUCTURE | COMPONENT_COUNT | | | NUMBER | |
| VM_HL7_MESSAGE_STRUCTURE | DEFAULT_VALUE | | | VARCHAR2 | |
| VM_HL7_MESSAGE_STRUCTURE | SEGMENT_REQUIRED | | | VARCHAR2 | |
| VM_HL7_MESSAGE_STRUCTURE | FIELD_REQUIRED | | | VARCHAR2 | |
| VM_HL7_MESSAGE_STRUCTURE | FIELD_FORMAT | | | VARCHAR2 | |
| VM_HL7_MESSAGE_STRUCTURE | FIELD_MAPPING | | | NUMBER | |
| VSAMPLE | PATIENT_ID | | | NUMBER | |
| VSAMPLE | VERMONT_SAMPLE | | | DATE | |
| WEB_PAGE | WEB_PAGE_URL | | | VARCHAR2 | |
| WEB_PAGE | DESCRIPTION | | | VARCHAR2 | |
| WEB_PAGE | WEB_PAGE_ACCESS | | | VARCHAR2 | |
| WEB_PAGE | APPLICATION_FLAG | | | VARCHAR2 | |
| WEEKLY_LAB_PROJECT_TOTALS | WEEK_ENDING | | | DATE | |
| WEEKLY_LAB_PROJECT_TOTALS | TOTAL_LOADED | | | NUMBER | |
| WEEKLY_LAB_TEST_TOTALS | LAB_ID | | | NUMBER | |
| WEEKLY_LAB_TEST_TOTALS | TEST_CODE | | | VARCHAR2 | |
| WEEKLY_LAB_TEST_TOTALS | WEEK_ENDING | | | DATE | |
| WEEKLY_LAB_TEST_TOTALS | STATUS | | | NUMBER | |
| WEEKLY_LAB_TEST_TOTALS | TOTAL_LOADED_BY_RESULT_DATE | | | NUMBER | |
| WEEKLY_LAB_TEST_TOTALS | TOTAL_LOADED_BY_PARSE_DATE | | | NUMBER | |
| WEEKLY_LAB_TEST_TOTALS | TOTAL_LOADED_BY_COLLECT_DATE | | | NUMBER | |
| WORKS_AT_HOUSES | LOCATION_ID | | | NUMBER | |
| WORKS_AT_HOUSES | CONTACT_ID | | | NUMBER | |

| | | FK | Descrip- | | Com- |
|---|---|---|---|---|---|
| Table Name | Column Name | Column | tion | Needed? | ments |
| ACCOUNT | ACCOUNT_ID | | | | |
| ACCOUNT | CONTRACT_STARTDATE | | | | |
| ACCOUNT | CONTRACT_ENDDATE | | | | |
| ACCOUNT | NAME | | | | |
| ACCOUNT | DESCRIPTION | | | | |
| ACCOUNT | REPORT_DISPLAY_NAME1 | | | | |
| ACCOUNT | REPORT_DISPLAY_NAME2 | | | | |
| ACCOUNT | CONTACT_ID | | | | |
| ACCOUNT | ACCOUNT_TYPE | | | | |
| ALT_PID_LOOKUP | | | | | |
| ALT_PID_LOOKUP | CODE | | | | |
| ALT_PID_LOOKUP | DESCRIPTION | | | | |
| ALT_PID_LOOKUP | ACTIVE | | | | |
| ALT_PID_LOOKUP | SORT_ORDER | | | | |
| CLIENT_LOOKUP | CODE | | | | |
| CLIENT_LOOKUP | DESCRIPTION | | | | |
| CLIENT_LOOKUP | CLIENT_TYPE | | | | |
| CLIENT_LOOKUP | ACTIVE | | | | |
| CLIENT_LOOKUP | SORT_ORDER | | | | |
| CODED_COLLECT_NOTE | COLLECT_NOTE | | | | |
| CODED_COLLECT_NOTE | NOTE_TEXT | | | | |
| CODED_COLLECT_NOTE | SERVICE_PROVIDER_ID | | | | |
| CODED_COLLECT_NOTE | NOTE_TYPE | | | | |
| CONTACT_LOOKUP | CODE | | | | |
| CONTACT_LOOKUP | DESCRIPTION | | | | |
| CONTACT_LOOKUP | CONTACT_TYPE | | | | |
| CONTACT_LOOKUP | ACTIVE | | | | |
| CONTACT_LOOKUP | SORT_ORDER | | | | |
| CONTROL_LIMIT | CONTROL_LIMIT_ID | | | | |
| CONTROL_LIMIT | TYPE1 | | | | |
| CONTROL_LIMIT | TYPE2 | | | | |
| CONTROL_LIMIT | CONTROL_LIMIT_NAME | | | | |
| CONTROL_LIMIT | DESCRIPTION | | | | |
| CONTROL_LIMIT_DATA | CONTROL_LIMIT_ID | | | | |
| CONTROL_LIMIT_DATA | DAY_OF_WEEK | | | | |
| CONTROL_LIMIT_DATA | LOWER_CONTROL_LIMIT | | | | |
| CONTROL_LIMIT_DATA | UPPER_CONTROL_LIMIT | | | | |
| CPT | CPT_CODE | | | | |
| CPT | DESCRIPTION | | | | |
| CPT | EFFECTIVE_DATE | | | | |
| DAYS_PERF_LOOKUP | DAYS_PERFORMED | | | | |
| DIABETES_TEST_OVERDUE_PERIODS | TEST_TYPE | | | | |
| DIABETES_TEST_OVERDUE_PERIODS | RESULT_RANGE | | | | |
| DIABETES_TEST_OVERDUE_PERIODS | REPORT_TYPE | | | | |
| DIABETES_TEST_OVERDUE_PERIODS | OVERDUE_PERIOD | | | | |
| DIABETES_TEST_REF_RANGE | LAB_ID | | | | |
| DIABETES_TEST_REF_RANGE | TEST_ID | | | | |
| DIABETES_TEST_REF_RANGE | LOW | | | | |
| DIABETES_TEST_REF_RANGE | HIGH | | | | |
| DIABETES_TEST_REF_RANGE | MEDIUM | | | | |
| DIABETES_TEST_REF_RANGE | LOW_INCLUSIVE | | | | |
| DIABETES_TEST_REF_RANGE | HIGH_INCLUSIVE | | | | |
| DIABETES_TEST_REF_RANGE | TEST_VERSION | | | | |
| ERROR_MESSAGES | ERROR_KEY | | | | |
| ERROR_MESSAGES | ERROR_MESSAGE | | | | |
| EVENT_LOOKUP | CODE | | | | |
| EVENT_LOOKUP | DESCRIPTION | | | | |
| EVENT_LOOKUP | EVENT_TYPE | | | | |
| EVENT_LOOKUP | ACTIVE | | | | |
| EVENT_LOOKUP | SORT_ORDER | | | | |
| FS_MC_TRUTH_TABLE | TEST_TYPE | | | | |
| FS_MC_TRUTH_TABLE | PREVIOUS_RESULT_RANGE | | | | |
| FS_MC_TRUTH_TABLE | LATEST_RESULT_RANGE | | | | |
| FS_MC_TRUTH_TABLE | OVERDUE_FLAG | | | | |
| FS_MC_TRUTH_TABLE | TEXT_SEQUENCE | | | | |
| FS_MC_TRUTH_TABLE | CANNED_TEXT_IDS | | | | |
| FTP_FILE_DATA | FILE_DATA_ID | | | | |
| FTP_FILE_DATA | FILE_NAME | | | | |
| FTP_FILE_DATA | FILE_STATUS | | | | |
| FTP_FILE_DATA | LAB_ID | | | | |
| FTP_FILE_DATA | FILE_TYPE | | | | |
| FTP_FILE_DATA | LOGGER_ID | | | | |
| FTP_FILE_DATA | TIMESTAMP | | | | |
| FTP_FILE_FIELD_MAPPING | LAB_ID | | | | |
| FTP_FILE_FIELD_MAPPING | FIELD_NAME | | | | |
| FTP_FILE_FIELD_MAPPING | POSITION | | | | |
| FTP_FILE_FIELD_MAPPING | REQUIRED | | | | |
| FTP_FILE_FIELD_MAPPING | DEFAULT_VALUE | | | | |
| FTP_FILE_FIELD_MAPPING | ACTIVE | | | | |
| FTP_FILE_FIELD_MAPPING | FORMAT | | | | |
| FTP_FILE_FIELD_MAPPING | FIELD_TYPE | | | | |
| GROUP_ACCESS | GROUP_NAME | | | | |
| GROUP_ACCESS | WEB_PAGE_URL | | | | |
| GROUP_ACCESS | APPLICATION_FLAG | | | | |
| GROUP_PRIVILEGE | CLIENT_ID | | | | |
| GROUP_PRIVILEGE | CONTACT_ID | | | | |
| GROUP_PRIVILEGE | GROUP_NAME | | | | |
| GROUP_PRIVILEGE | APPLICATION_FLAG | | | | |
| HAS_ROLE | ROLE_ID | | | | |
| HAS_ROLE | CONTACT_ID | | | | |
| HISTORY_LOG | DATETIME | | | | |
| HISTORY_LOG | PATIENT | | | | |
| HISTORY_LOG | PHYSICIAN | | | | |
| HISTORY_LOG | PRACTICE | | | | |
| HISTORY_LOG | ERRCODE | | | | |
| HISTORY_LOG | ERRMSG | | | | |
| HISTORY_LOG | LOCATION | | | | |
| HISTORY_LOG | LAB | | | | |
| HISTORY_LOG | TEST_RESULT | | | | |
| ICD9 | ICD9_CODE | | | | |
| ICD9 | SHORT_DESCRIPTION | | | | |
| ICD9 | LONG_DESCRIPTION | | | | |
| ICD9 | ICD9_SPECIFICITY | | | | |
| ICD9_NEVER_COVERS | INSURANCE_PLAN_ID | | | | |
| ICD9_NEVER_COVERS | COVERAGE_POLICY_ID | | | | |
| ICD9_NEVER_COVERS | ICD9_CODE | | | | |
| ICD9_NEVER_COVERS | COVERAGE_COMMENT_CODE | | | | |
| ICD9_NEVER_COVERS | NC_EFFECTIVE_DATE | | | | |
| ICD9_NEVER_COVERS | NC_ACTIVE_STATUS | | | | |
| ICD9_PARENT_CODE | ICD9_CODE | | | | |
| ICD9_PARENT_CODE | SHORT_DESCRIPTION | | | | |
| ICD9_PARENT_CODE | LONG_DESCRIPTION | | | | |
| ICD9_PARENT_CODE | ICD9_SPECIFICITY | | | | |
| ICD9_PARENT_CODE | PARENT_CODE | | | | |
| KEYMASTER | KEYMASTER_TABLE | | | | |
| KEYMASTER | KEYMASTER_KEY | | | | |
| LOGGER | LOGGER_ID | | | | |
| LOGGER | LOGGER_TIMESTAMP | | | | |
| LOGGER | SEVERITY | | | | |
| LOGGER | LOGGER_SOURCE | | | | |
| LOGGER | DETAILED_SOURCE | | | | |
| LOGGER | DESCRIPTION | | | | |
| LOGGER | HL7_MESSAGE_ID | | | | |
| LOGGER | HL7_MESSAGE_TYPE | | | | |
| LOGGER | LOGGER_STATUS | | | | |
| LOGGER | RESTART_POINT | | | | |
| LOGGER | CONTROL_ID | | | | |
| LOGGER_LOOKUP | CODE | | | | |
| LOGGER_LOOKUP | DESCRIPTION | | | | |
| LOGGER_LOOKUP | ACTIVE | | | | |
| LOGGER_LOOKUP | LOGGER_LOOKUP_TYPE | | | | |
| LOGGER_LOOKUP | SORT_ORDER | | | | |
| LOGGER_MONITOR_CONTROL | LAST_MESSAGE_SENT | | | | |
| LOGIN_VERIFICATION | CONTACT_ID | | | | |
| LOGIN_VERIFICATION | PASSWORD_EXPIRY_DATE | | | | |
| LOGIN_VERIFICATION | IS_ACTIVE | | | | |
| LOGIN_VERIFICATION | CURRENT_PASSWORD | | | | |
| LOGIN_VERIFICATION | PREV_PASSWORD_1 | | | | |
| LOGIN_VERIFICATION | PREV_PASSWORD_2 | | | | |
| LOGIN_VERIFICATION | PREV_PASSWORD_3 | | | | |
| LOGIN_VERIFICATION | PREV_PASSWORD_4 | | | | |
| LOGIN_VERIFICATION | PREV_PASSWORD_5 | | | | |
| NAVIGATIONHTML | NAVIGATIONHTML_ID | | | | |
| NAVIGATIONHTML | DESCRIPTION | | | | |
| NAVIGATIONHTML | URL | | | | |
| NAVIGATIONHTML | INDENT | | | | |
| NAVIGATIONHTML | MODULE | | | | |
| NAVIGATIONHTML | SORTORDER | | | | |
| NAVIGATIONHTML | NAVIGATIONHTML_MASTER | | | | |
| NAVIGATIONHTML | NAVIGATIONHTML_LEVEL | | | | |
| NAVIGATIONHTML | PAGE | | | | |
| NAVIGATIONHTML | APPLICATION_FLAG | | | | |
| NON_RESULT_VALUES | NON_RESULT_ID | | | | |
| NON_RESULT_VALUES | SERVICE_PROVIDER_ID | | | | |
| NON_RESULT_VALUES | NON_RESULT_VALUE | | | | |
| NON_RESULT_VALUES | REJECT_IF_FOUND_FLAG | | | | |
| NON_RESULT_VALUES | NOTIFY_IF_FOUND_FLAG | | | | |
| NON_RESULT_VALUES | STATUS | | | | |
| ONTIME_ABC_CALCULATOR | SUM_PATIENT_NUM | | | | |
| ONTIME_ABC_CALCULATOR | PATIENT_NUM | | | | |
| ONTIME_ABC_CALCULATOR | ONTIME_PATIENT_NUM | | | | |
| ONTIME_ABC_CALCULATOR | BAYESIAN_APF | | | | |
| ONTIME_ABC_CALCULATOR | CONTACT_ID | | | | |
| PARSED_RESULTS | PARSED_RESULT_ID | | | | |
| PARSED_RESULTS | BATCH_ID | | | | |
| PARSED_RESULTS | ORGANIZATION_ID | | | | |
| PARSED_RESULTS | LIS_ACCESSION_NUMBER | | | | |
| PARSED_RESULTS | SERVICE_ORDER_ID | | | | |
| PARSED_RESULTS | PATIENT_ID | | | | |
| PARSED_RESULTS | PATIENT_FIRST_NAME | | | | |
| PARSED_RESULTS | PATIENT_LAST_NAME | | | | |
| PARSED_RESULTS | DOB | | | | |
| PARSED_RESULTS | SEX | | | | |
| PARSED_RESULTS | ADDRESS1 | | | | |
| PARSED_RESULTS | ADDRESS2 | | | | |
| PARSED_RESULTS | CITY | | | | |
| PARSED_RESULTS | STATE | | | | |
| PARSED_RESULTS | ZIP | | | | |
| PARSED_RESULTS | ORDERED_TEST_CODE | | | | |
| PARSED_RESULTS | RESULTED_TEST_CODE | | | | |
| PARSED_RESULTS | NUMERIC_RESULT | | | | |
| PARSED_RESULTS | TEXT_RESULT | | | | |
| PARSED_RESULTS | RESULT_DATE | | | | |
| PARSED_RESULTS | PROVIDER_ID | | | | |
| PARSED_RESULTS | PROVIDER_FIRST_NAME | | | | |
| PARSED_RESULTS | PROVIDER_LAST_NAME | | | | |
| PARSED_RESULTS | VALID_FLAG | | | | |
| PARSED_RESULTS | PARSE_DATE | | | | |
| PARSED_RESULTS | FILED_FLAG | | | | |
| PARSED_RESULTS | PATIENT_IDENTIFIER | | | | |
| PARSED_RESULTS | MESSAGE | | | | |
| PARSED_RESULTS | CLIENT_ID | | | | |
| PARSED_RESULTS | PROVIDER_IDENTIFIER | | | | |
| PARSED_RESULTS | LIS_COLLECT_DATE | | | | |
| PARSED_RESULTS | LIS_RECEIVED_DATE | | | | |
| PARSED_RESULTS | LIS_COLLECT_TIME | | | | |
| PARSED_RESULTS | LIS_RECEIVED_TIME | | | | |
| PARSED_RESULTS | RESULT_TIME | | | | |
| PARSED_RESULTS | RESULT_RANGE | | | | |
| PATIENT_ACCOUNT | PATIENT_ID | | | | |
| PATIENT_ACCOUNT | PATIENT_ACCOUNT_STATUS | | | | |
| PATIENT_ACCOUNT | ACCOUNT_ID | | | | |
| PATIENT_ACCOUNT_CHANGE_HISTORY | DATE_TIME | | | | |
| PATIENT_ACCOUNT_CHANGE_HISTORY | PATIENT_ID | | | | |
| PATIENT_ACCOUNT_CHANGE_HISTORY | ACCOUNT_ID | | | | |
| PATIENT_ACCOUNT_CHANGE_HISTORY | LATEST_STATUS | | | | |
| PATIENT_ACCOUNT_CHANGE_HISTORY | REASON_FOR_CHANGE | | | | |
| PATIENT_ACCOUNT_STATUS_LOOKUP | PATIENT_ACCOUNT_STATUS | | | | |
| PATIENT_ACCOUNT_STATUS_LOOKUP | STATUS_NAME | | | | |
| PATIENT_ALIAS | LAST_NAME | | | | |
| PATIENT_ALIAS | FIRST_NAME | | | | |
| PATIENT_ALIAS | MIDDLE_NAME | | | | |
| PATIENT_ALIAS | PATIENT_ID | | | | |
| PATIENT_STATUS | PATIENT_ID | | | | |
| PATIENT_STATUS | PATIENT_STATUS_ID | | | | |
| PATIENT_STATUS | RESEARCH | | | | |
| PATIENT_STATUS | QUALITY_IMPROVEMENT | | | | |
| PAYOR_ACCOUNT | ORGANIZATION_ID | | | | |
| PAYOR_ACCOUNT | ACCOUNT_ID | | | | |
| PAYOR_ACCOUNT | NAME | | | | |
| PAYOR_ACCOUNT | DESCRIPTION | | | | |
| PAYOR_ACCOUNT | CONTACT_ID | | | | |
| PETHNIC_LOOKUP | CODE | | | | |
| PETHNIC_LOOKUP | DESCRIPTION | | | | |
| PETHNIC_LOOKUP | ACTIVE | | | | |
| PETHNIC_LOOKUP | SORT_ORDER | | | | |
| PGUAR_REL_LOOKUP | CODE | | | | |
| PGUAR_REL_LOOKUP | DESCRIPTION | | | | |
| PGUAR_REL_LOOKUP | ACTIVE | | | | |
| PGUAR_REL_LOOKUP | SORT_ORDER | | | | |
| PHYSICIAN_SAMPLE_PERCENTAGE | LOW_PATIENT_PERCENTAGE | | | | |
| PHYSICIAN_SAMPLE_PERCENTAGE | MEDIUM_PATIENT_PERCENTAGE | | | | |
| PHYSICIAN_SAMPLE_PERCENTAGE | HIGH_PATIENT_PERCENTAGE | | | | |
| PHYSICIAN_SAMPLE_PERCENTAGE | TEST_CODE | | | | |
| PHYSICIAN_SAMPLE_PERCENTAGE | PRIMARY_PROVIDER | | | | |
| PHYS_STAT_LOOKUP | PHYS_STATUS | | | | |
| PHYS_STAT_LOOKUP | DESCRIPTION | | | | |
| PL_ACCOUNT | CLIENT_ID | | | | |
| PL_ACCOUNT | ACCOUNT_NUMBER | | | | |
| PL_ACCOUNT | ACCOUNT_NAME | | | | |
| PL_ACCOUNT | CONTRACT_STARTDATE | | | | |
| PL_ACCOUNT | CONTRACT_ENDDATE | | | | |
| PL_ACCOUNT | DEFAULT_DISCOUNT | | | | |
| PL_ACCOUNT | FEE_SCHEDULE_ID | | | | |
| PL_ACCOUNT | RANK | | | | |
| PL_ACCOUNT | CHECK_COMPLIANCE | | | | |
| PL_ACCOUNT | SERVICE_PROVIDER_ID | | | | |
| PL_CONTAINER | CONTAINER_TYPE | | | | |
| PL_CONTAINER | MAX_VOLUME | | | | |
| PL_CONTAINER | PRESERVATIVE_TYPE | | | | |
| PL_CONTAINER | UNIT_TYPE | | | | |
| PL_CONTAINER | SHORTNAME | | | | |
| PL_GROUP | GROUP_NAME | | | | |
| PL_GROUP | GROUP_DESCRIPTION | | | | |
| PL_GROUP | GROUP_TYPE | | | | |
| PL_GROUP | APPLICATION_FLAG | | | | |
| PL_LOCATION | LOCATION_ID | | | | |
| PL_LOCATION | LOCATION_NAME | | | | |
| PL_LOCATION | CODE | | | | |
| PL_LOCATION | ADDRESS_1 | | | | |
| PL_LOCATION | ADDRESS_2 | | | | |
| PL_LOCATION | CITY | | | | |
| PL_LOCATION | STATE | | | | |
| PL_LOCATION | ZIP | | | | |
| PL_LOCATION | COUNTRY | | | | |
| PL_LOCATION | PHONE | | | | |
| PL_LOCATION | PHONE_2 | | | | |
| PL_LOCATION | FAX | | | | |
| PL_LOCATION | HOURS | | | | |
| PL_LOCATION | SUPPLY_DIST | | | | |
| PL_LOCATION_CHANGE_HISTORY | CREATE_TIMESTAMP | | | | |
| PL_LOCATION_CHANGE_HISTORY | LOCATION_ID | | | | |
| PL_LOCATION_CHANGE_HISTORY | LOCATION_NAME | | | | |
| PL_LOCATION_CHANGE_HISTORY | CODE | | | | |
| PL_LOCATION_CHANGE_HISTORY | ADDRESS_1 | | | | |
| PL_LOCATION_CHANGE_HISTORY | ADDRESS_2 | | | | |
| PL_LOCATION_CHANGE_HISTORY | CITY | | | | |
| PL_LOCATION_CHANGE_HISTORY | STATE | | | | |
| PL_LOCATION_CHANGE_HISTORY | ZIP | | | | |
| PL_LOCATION_CHANGE_HISTORY | COUNTRY | | | | |
| PL_LOCATION_CHANGE_HISTORY | PHONE | | | | |
| PL_LOCATION_CHANGE_HISTORY | PHONE_2 | | | | |
| PL_LOCATION_CHANGE_HISTORY | FAX | | | | |
| PL_LOCATION_CHANGE_HISTORY | HOURS | | | | |
| PL_LOCATION_CHANGE_HISTORY | SUPPLY_DIST | | | | |
| PL_LOCATION_CHANGE_HISTORY | REASON_FOR_CHANGE | | | | |
| PL_LOCATION_CHANGE_HISTORY | LATEST_STATUS | | | | |
| PL_ORGANIZATION | ORGANIZATION_ID | | | | |
| PL_ORGANIZATION | ORGANIZATION_NAME | | | | |
| PL_ORGANIZATION | LOCATION_ID | | | | |
| PL_ORGANIZATION | IS_CLIENT | | | | |
| PL_ORGANIZATION | IS_SERVICE_PROVIDER | | | | |
| PL_ORGANIZATION | IS_PARENT | | | | |
| PL_ORGANIZATION | IS_NETWORK | | | | |
| PL_PROCEDURE | PROC_CODE | | | | |
| PL_PROCEDURE | PROCEDURE_NAME | | | | |
| PL_PROCEDURE | SHORT_NAME | | | | |
| PL_PROCEDURE | MODALITY | | | | |
| PL_PROCEDURE | ALPHA_NAME | | | | |
| PL_PROCEDURE | PROC_NOTE | | | | |
| PL_PROCEDURE | CPT_CODE | | | | |
| PL_PROCEDURE | PROC_VERSION | | | | |
| PL_PROCEDURE | PROFILE_VERSION | | | | |
| PL_PROCEDURE | TEST_VERSION | | | | |
| PL_PROCEDURE | PROC_EFFECTIVE_DATE | | | | |
| PL_PROCEDURE | PROC_ACTIVE_STATUS | | | | |
| PL_ROLE | ROLE_ID | | | | |
| PL_ROLE | ROLE_DESCRIPTION | | | | |
| PROVIDER_ACCOUNT | SERVICE_PROVIDER_ID | | | | |
| PROVIDER_ACCOUNT | ACCOUNT_NUMBER | | | | |
| PSEX_LOOKUP | CODE | | | | |
| PSEX_LOOKUP | DESCRIPTION | | | | |
| PSEX_LOOKUP | ACTIVE | | | | |
| PSEX_LOOKUP | SORT_ORDER | | | | |
| PSTATUS_LOOKUP | CODE | | | | |
| PSTATUS_LOOKUP | DESCRIPTION | | | | |
| PSTATUS_LOOKUP | ACTIVE | | | | |
| PSTATUS_LOOKUP | SORT_ORDER | | | | |
| REFERENCE_RANGE | SERVICE_PROVIDER_ID | | | | |
| REFERENCE_RANGE | SERVICE_CODE | | | | |
| REFERENCE_RANGE | RANGE_ID | | | | |
| REFERENCE_RANGE | EFFECTIVE_DATE | | | | |
| REFERENCE_RANGE | INACTIVE_DATE | | | | |
| REFERENCE_RANGE | RANGE_STATUS | | | | |
| REFERENCE_RANGE | AGE | | | | |
| REFERENCE_RANGE | SEX | | | | |
| REFERENCE_RANGE | PHYS_STATUS | | | | |
| REFERENCE_RANGE | UNIT | | | | |
| REFERENCE_RANGE | CRITICAL_LOW | | | | |
| REFERENCE_RANGE | CRITICAL_HIGH | | | | |
| REFERENCE_RANGE | NORMAL_LOW | | | | |
| REFERENCE_RANGE | NORMAL_HIGH | | | | |
| REFERENCE_RANGE | TEXTUAL_NORMAL | | | | |
| REFERENCE_RANGE | DELTA | | | | |
| REFERENCE_RANGE | SORT_ORDER | | | | |
| REFERENCE_UNITS | UNIT | | | | |
| REFERENCE_UNITS | DESCRIPTION | | | | |
| REMINDER_TRUTH_TABLE | TEST_TYPE | | | | |
| REMINDER_TRUTH_TABLE | REMINDER_TYPE | | | | |
| REMINDER_TRUTH_TABLE | RESULT_RANGE | | | | |
| REMINDER_TRUTH_TABLE | CANNED_TEXT_IDS | | | | |
| REMINDER_TRUTH_TABLE_TMP | TEST_TYPE | | | | |
| REMINDER_TRUTH_TABLE_TMP | REMINDER_TYPE | | | | |
| REMINDER_TRUTH_TABLE_TMP | RESULT_RANGE | | | | |
| REMINDER_TRUTH_TABLE_TMP | SEQUENCE_NO | | | | |
| REMINDER_TRUTH_TABLE_TMP | CANNED_TEXT_IDS | | | | |
| REPORT | REPORT_NUMBER | | | | |
| REPORT | REPORT_NAME | | | | |
| REPORT | REPORT_TYPE | | | | |
| REPORT | ORGANIZATION_ID | | | | |
| REPORT_DATA | REPORT_DATA_ID | | | | |
| REPORT_DATA | OUTPUT_TYPE_ID | | | | |
| REPORT_DATA | PRACTICE_ID | | | | |
| REPORT_DATA | FILE_NAME | | | | |
| REPORT_DATA | CONTACT_ID | | | | |
| REPORT_DATA | REPORT_STATUS | | | | |
| REPORT_DATA | OUTPUT_DATE | | | | |
| RESULT_ABC_CALCULATOR | SUM_PATIENT_NUM | | | | |
| RESULT_ABC_CALCULATOR | PATIENT_NUM | | | | |
| RESULT_ABC_CALCULATOR | LOW_PATIENT_NUM | | | | |
| RESULT_ABC_CALCULATOR | MEDIUM_PATIENT_NUM | | | | |
| RESULT_ABC_CALCULATOR | HIGH_PATIENT_NUM | | | | |
| RESULT_ABC_CALCULATOR | BAYESIAN_APF | | | | |
| RESULT_ABC_CALCULATOR | CONTACT_ID | | | | |
| RESULT_ABC_CALCULATOR | TEST_ID | | | | |
| RESULT_ABC_CALCULATOR_2 | SUM_PATIENT_NUM | | | | |
| RESULT_ABC_CALCULATOR_2 | PATIENT_NUM | | | | |
| RESULT_ABC_CALCULATOR_2 | LOW_PATIENT_NUM | | | | |
| RESULT_ABC_CALCULATOR_2 | MEDIUM_PATIENT_NUM | | | | |
| RESULT_ABC_CALCULATOR_2 | HIGH_PATIENT_NUM | | | | |
| RESULT_ABC_CALCULATOR_2 | BAYESIAN_APF | | | | |
| RESULT_ABC_CALCULATOR_2 | CONTACT_ID | | | | |
| RESULT_ABC_CALCULATOR_2 | TEST_ID | | | | |
| RESULT_ABC_STEP | PATIENT_ID | | | | |
| RESULT_ABC_STEP | RANGE_TYPE | | | | |
| RESULT_ABC_STEP | CONTACT_ID | | | | |
| RESULT_ABC_STEP | TEST_ID | | | | |
| RESULT_LOOKUP | CODE | | | | |
| RESULT_LOOKUP | DESCRIPTION | | | | |
| RESULT_LOOKUP | RESULT_TYPE | | | | |
| RESULT_LOOKUP | ACTIVE | | | | |
| RESULT_LOOKUP | SORT_ORDER | | | | |
| RESULT_LOOKUP | PRIORITY | | | | |
| RESULTS_SOURCE | ROW_KEY | | | | |
| RESULTS_SOURCE | MRN | | | | |
| RESULTS_SOURCE | LNAME | | | | |
| RESULTS_SOURCE | FNAME | | | | |
| RESULTS_SOURCE | MI | | | | |
| RESULTS_SOURCE | DOB | | | | |
| RESULTS_SOURCE | SEX | | | | |
| RESULTS_SOURCE | LIS_COLLECT_DATE | | | | |
| RESULTS_SOURCE | LIS_ACCESSION_NUM | | | | |
| RESULTS_SOURCE | SERVICE_CODE | | | | |
| RESULTS_SOURCE | PARENT_CODE | | | | |
| RESULTS_SOURCE | ORDERING_PROV_ID | | | | |
| RESULTS_SOURCE | ORDERING_CLIENT | | | | |
| RESULTS_SOURCE | PERFORMED_AT | | | | |
| RESULTS_SOURCE | UNIT | | | | |
| RESULTS_SOURCE | TEST_RESULT_DETAIL_NUMERIC | | | | |
| RESULTS_SOURCE | TEXT | | | | |
| RESULTS_SOURCE | LIS_RECEIVED_DATE | | | | |
| RESULTS_SOURCE | RESULT_DATE | | | | |
| SAMPLE_PERCENTAGE_MLDL | PATIENT_ID | | | | |
| SAMPLE_PERCENTAGE_MLDL | LOW_PATIENTS | | | | |
| SAMPLE_PERCENTAGE_MLDL | MEDIUM_PATIENTS | | | | |
| SAMPLE_PERCENTAGE_MLDL | HIGH_PATIENTS | | | | |
| SAMPLE_PERCENTAGE_MLDL | CONTACT_ID | | | | |
| SERVICE_DIAGNOSIS | SERVICE_ORDER_ID | | | | |
| SERVICE_DIAGNOSIS | ICD9_CODE | | | | |
| SERVICE_RULES | SERVICE_CODE | | | | |
| SERVICE_RULES | SERVICE_RULE | | | | |
| SERVICE_RULES | SERVICE_RULE_TYPE | | | | |
| SITE_METADATA | METADATA_ID | | | | |
| SITE_METADATA | ORGANIZATION_ID | | | | |
| SITE_METADATA | METADATA_VALUE | | | | |
| SOFTWARE_RELEASE | RELEASE_ID | | | | |
| SOFTWARE_RELEASE | SOFTWARE_RELEASE_DATE | | | | |
| SOFTWARE_RELEASE | RELEASE_NOTES | | | | |
| TEST_COLLECTION | SERVICE_PROVIDER_ID | | | | |
| TEST_COLLECTION | SERVICE_CODE | | | | |
| TEST_COLLECTION | COLLECTION_GROUP_ID | | | | |
| TEST_COLLECTION | SOURCE_TYPE | | | | |
| TEST_COLLECTION | CONTAINER_TYPE | | | | |
| TEST_COLLECTION | RELATION | | | | |
| TEST_COLLECTION | PREFERRED | | | | |
| TEST_COLLECTION | CONSOLIDATION_FLAG | | | | |
| TEST_COLLECTION | CONTAINER_QTY | | | | |
| TEST_COLLECTION | VOLUME | | | | |
| TEST_COLLECTION | MIN_VOLUME | | | | |
| TEST_COLLECTION | UNIT_TYPE | | | | |
| TEST_COLLECTION | COLLECT_NOTE | | | | |
| TEST_COLLECTION | EFFECTIVE_DATE | | | | |
| TEST_COLLECTION | CLIENT_STORAGE | | | | |
| TEST_COLLECTION | LAB_STORAGE | | | | |
| TEST_COLLECTION | PROCESSING_REQUIRED | | | | |
| TEST_COLLECTION | TEST_COLLECTION_VERSION | | | | |
| TEST_LOOKUP | TEST_ID | | | | |
| TEST_LOOKUP | TEST_NAME | | | | |
| TEST_ORDER_INFO | SERVICE_ORDER_ID | | | | |
| TEST_ORDER_INFO | SERVICE_PROVIDER_ID | | | | |
| TEST_ORDER_INFO | SERVICE_CODE | | | | |
| TEST_ORDER_INFO | COLLECT_DATE | | | | |
| TEST_ORDER_INFO | SOURCE_TYPE | | | | |
| TEST_ORDER_INFO | COLLECT_TEXT | | | | |
| TEST_ORDER_PROFILE | SERVICE_ORDER_ID | | | | |
| TEST_ORDER_PROFILE | ORDERED_SERVICE_PROVIDER_ID | | | | |
| TEST_ORDER_PROFILE | ORDERED_SERVICE_CODE | | | | |
| TEST_ORDER_PROFILE | PARENT_SERVICE_PROVIDER_ID | | | | |
| TEST_ORDER_PROFILE | PARENT_SERVICE_CODE | | | | |
| TEST_ORDER_PROFILE | CHILD_SERVICE_PROVIDER_ID | | | | |
| TEST_ORDER_PROFILE | CHILD_SERVICE_CODE | | | | |
| TEST_ORDER_PROFILE | PARENT_SORT_ORDER | | | | |
| TEST_ORDER_PROFILE | CHILD_SORT_ORDER | | | | |
| TEST_ORDER_PROFILE | PARENT_VERSION | | | | |
| TEST_ORDER_PROFILE | CHILD_VERSION | | | | |
| TEST_ORDER_PROFILE | RESULTABLE | | | | |
| TEST_ORDER_PROFILE | PERFORM_ORDERED_SERV_PROV_ID | | | | |
| TEST_ORDER_PROFILE | PERFORM_ORDERED_SERVICE_CODE | | | | |
| TEST_ORDER_PROFILE | PERFORM_PARENT_SERV_PROV_ID | | | | |
| TEST_ORDER_PROFILE | PERFORM_PARENT_SERVICE_CODE | | | | |
| TEST_ORDER_PROFILE | PERFORM_CHILD_SERV_PROV_ID | | | | |
| TEST_ORDER_PROFILE | PERFORM_CHILD_SERVICE_CODE | | | | |
| TEST_RESULT_INTERP | LAB_ID | | | | |
| TEST_RESULT_INTERP | TEST_CODE | | | | |
| TEST_RESULT_INTERP | TEXT | | | | |
| TEST_RESULT_INTERP | INTERP_TEXT | | | | |
| TEST_RESULT_INTERP | DISABLE_REMINDER | | | | |
| TRANSLATION_TABLE | TEST_TYPE | | | | |
| TRANSLATION_TABLE | REMINDER_TYPE | | | | |
| TRANSLATION_TABLE | CANNED_TEXT_IDS | | | | |
| UNIT_TYPE | UNIT_TYPE | | | | |
| UNIT_TYPE | UNIT_TYPE_DESCRIPTION | | | | |
| UNIT_TYPE | BASE_UNIT_CONVERSION | | | | |
| UNIT_TYPE | BASE_UNIT_TYPE | | | | |
| V_LAB | SERVICE_PROVIDER_ID | | | | |
| V_LAB | SERVICE_PROVIDER_NAME | | | | |
| V_LAB | LOCATION_ID | | | | |
| V_LAB | ADDRESS_1 | | | | |
| V_LAB | ADDRESS_2 | | | | |
| V_LAB | CITY | | | | |
| V_LAB | STATE | | | | |
| V_LAB | COUNTRY | | | | |
| V_LAB | ZIP | | | | |
| V_LAB | PHONE | | | | |
| V_LAB | PHONE_2 | | | | |
| V_LAB | FAX | | | | |
| V_LAB | HOURS | | | | |
| V_ORDERED_PARENT_RESULTED | ORDERED | | | | |
| V_ORDERED_PARENT_RESULTED | PARENT | | | | |
| V_ORDERED_PARENT_RESULTED | RESULTED | | | | |
| V_ORDERED_PARENT_RESULTED | SERVICE_CODE | | | | |
| V_ORDERED_PARENT_RESULTED | TEST_CODE | | | | |
| V_ORDERED_PARENT_RESULTED | SERVICE_PROVIDER_ID | | | | |
| V_ORDERED_PARENT_RESULTED | SORT_ORDER | | | | |
| V_PATIENT | PATIENT_ID | | | | |
| V_PATIENT | LAST_NAME | | | | |
| V_PATIENT | FIRST_NAME | | | | |
| V_PATIENT | MIDDLE_NAME | | | | |
| V_PATIENT | DOB | | | | |
| V_PATIENT | PREFIX | | | | |
| V_PATIENT | SUFFIX | | | | |
| V_PATIENT | SPOUSE | | | | |
| V_PATIENT | LAST_MESSAGE_ID | | | | |
| V_PATIENT | GUARANTOR | | | | |
| V_PATIENT | EMPLOYER_SCHOOL | | | | |
| V_PATIENT | PATIENT_HOME_LOCATION | | | | |
| V_PATIENT | CURRENT_PROVIDER | | | | |
| V_PATIENT | PRIMARY_PROVIDER | | | | |
| V_PATIENT | DATE_OF_DEATH | | | | |
| V_PATIENT | SEX | | | | |
| V_PATIENT | GUAR_RELATION | | | | |
| V_PATIENT | MARITAL_STATUS | | | | |
| V_PATIENT | ETHNIC_GROUP | | | | |
| V_PATIENT | SPECIES | | | | |
| V_PATIENT | STATUS | | | | |
| V_PATIENT | DATE_TIME | | | | |
| V_PATIENT | PATIENT_STATUS_ID | | | | |
| V_PATIENT_1 | STATUS | | | | |
| V_PATIENT_1 | DATE_TIME | | | | |
| V_PATIENT_1 | PATIENT_STATUS_ID | | | | |
| V_PATIENT_1 | PATIENT_ID | | | | |
| V_PATIENT_1 | LAST_NAME | | | | |
| V_PATIENT_1 | FIRST_NAME | | | | |
| V_PATIENT_1 | MIDDLE_NAME | | | | |
| V_PATIENT_1 | DOB | | | | |
| V_PATIENT_1 | PREFIX | | | | |
| V_PATIENT_1 | SUFFIX | | | | |
| V_PATIENT_1 | SPOUSE | | | | |
| V_PATIENT_1 | LAST_MESSAGE_ID | | | | |
| V_PATIENT_1 | GUARANTOR | | | | |
| V_PATIENT_1 | EMPLOYER_SCHOOL | | | | |
| V_PATIENT_1 | PATIENT_HOME_LOCATION | | | | |
| V_PATIENT_1 | CURRENT_PROVIDER | | | | |
| V_PATIENT_1 | PRIMARY_PROVIDER | | | | |
| V_PATIENT_1 | DATE_OF_DEATH | | | | |
| V_PATIENT_1 | SEX | | | | |
| V_PATIENT_1 | GUAR_RELATION | | | | |
| V_PATIENT_1 | MARITAL_STATUS | | | | |
| V_PATIENT_1 | ETHNIC_GROUP | | | | |
| V_PATIENT_1 | SPECIES | | | | |
| V_PATIENT_2 | PATIENT_ID | | | | |
| V_PATIENT_2 | LAST_NAME | | | | |
| V_PATIENT_2 | FIRST_NAME | | | | |
| V_PATIENT_2 | MIDDLE_NAME | | | | |
| V_PATIENT_2 | DOB | | | | |
| V_PATIENT_2 | PREFIX | | | | |
| V_PATIENT_2 | SUFFIX | | | | |
| V_PATIENT_2 | SPOUSE | | | | |
| V_PATIENT_2 | LAST_MESSAGE_ID | | | | |
| V_PATIENT_2 | GUARANTOR | | | | |
| V_PATIENT_2 | EMPLOYER_SCHOOL | | | | |
| V_PATIENT_2 | PATIENT_HOME_LOCATION | | | | |
| V_PATIENT_2 | CURRENT_PROVIDER | | | | |
| V_PATIENT_2 | PRIMARY_PROVIDER | | | | |
| V_PATIENT_2 | DATE_OF_DEATH | | | | |
| V_PATIENT_2 | SEX | | | | |
| V_PATIENT_2 | GUAR_RELATION | | | | |
| V_PATIENT_2 | MARITAL_STATUS | | | | |
| V_PATIENT_2 | ETHNIC_GROUP | | | | |
| V_PATIENT_2 | SPECIES | | | | |
| V_PATIENT_2 | STATUS | | | | |
| V_PATIENT_2 | PATIENT_STATUS_ID | | | | |
| V_PATIENT_STATUS | | | | | |
| V_PATIENT_STATUS | PATIENT_ID | | | | |
| V_PATIENT_STATUS | LAST_NAME | | | | |
| V_PATIENT_STATUS | FIRST_NAME | | | | |
| V_PATIENT_STATUS | MIDDLE_NAME | | | | |
| V_PATIENT_STATUS | DOB | | | | |
| V_PATIENT_STATUS | PREFIX | | | | |
| V_PATIENT_STATUS | SUFFIX | | | | |
| V_PATIENT_STATUS | SPOUSE | | | | |
| V_PATIENT_STATUS | LAST_MESSAGE_ID | | | | |
| V_PATIENT_STATUS | GUARANTOR | | | | |
| V_PATIENT_STATUS | EMPLOYER_SCHOOL | | | | |
| V_PATIENT_STATUS | PATIENT_HOME_LOCATION | | | | |
| V_PATIENT_STATUS | CURRENT_PROVIDER | | | | |
| V_PATIENT_STATUS | PRIMARY_PROVIDER | | | | |
| V_PATIENT_STATUS | DATE_OF_DEATH | | | | |
| V_PATIENT_STATUS | SEX | | | | |
| V_PATIENT_STATUS | GUAR_RELATION | | | | |
| V_PATIENT_STATUS | MARITAL_STATUS | | | | |
| V_PATIENT_STATUS | ETHNIC_GROUP | | | | |
| V_PATIENT_STATUS | SPECIES | | | | |
| V_PATIENT_STATUS | STATUS | | | | |
| V_PATIENT_STATUS | PATIENT_STATUS_ID | | | | |
| V_PHYSICIAN | CONTACT_ID | | | | |
| V_PHYSICIAN | LAST_NAME | | | | |
| V_PHYSICIAN | FIRST_NAME | | | | |
| V_PHYSICIAN | MIDDLE_NAME | | | | |
| V_PHYSICIAN | PREFIX | | | | |
| V_PHYSICIAN | SUFFIX | | | | |
| V_PHYSICIAN | TITLE | | | | |
| V_PHYSICIAN | WORK_PHONE | | | | |
| V_PHYSICIAN | HOME_PHONE | | | | |
| V_PHYSICIAN | MOBILE_PHONE | | | | |
| V_PHYSICIAN | FAX | | | | |
| V_PHYSICIAN | EMAIL | | | | |
| V_PHYSICIAN | USERNAME | | | | |
| V_PHYSICIAN | PIN | | | | |
| V_PHYSICIAN | CLIENT_ID | | | | |
| V_PHYSICIAN | EMPLOYMENT_DATE | | | | |
| V_PHYSICIAN | TERMINATION_DATE | | | | |
| V_PHYSICIAN | STATUS | | | | |
| V_PHYSICIAN | LOCATION_NAME | | | | |
| V_PHYSICIAN | ADDRESS_1 | | | | |
| V_PHYSICIAN | ADDRESS_2 | | | | |
| V_PHYSICIAN | CITY | | | | |
| V_PHYSICIAN | STATE | | | | |
| V_PHYSICIAN | ZIP | | | | |
| V_PHYSICIAN | COUNTRY | | | | |
| V_PHYSICIAN | LOC_PHONE | | | | |
| V_PHYSICIAN | LOC_FAX | | | | |
| V_PKG_BAT_TEST | PKG | | | | |
| V_PKG_BAT_TEST | BATTERY | | | | |
| V_PKG_BAT_TEST | TEST | | | | |
| V_PKG_BAT_TEST | SERVICE_CODE | | | | |
| V_PKG_BAT_TEST | TEST_CODE | | | | |
| V_PKG_BAT_TEST | SERVICE_PROVIDER_ID | | | | |
| V_PRACTICE | CLIENT_ID | | | | |
| V_PRACTICE | CLIENT_NAME | | | | |
| V_PRACTICE | ADDED_DATE | | | | |
| V_PRACTICE | CONTACT_ID | | | | |
| V_PRACTICE | STATUS | | | | |
| V_PRACTICE | PHYSICIAN_REFRACTORY_PERIOD | | | | |
| V_PRACTICE | PATIENT_REFRACTORY_PERIOD | | | | |
| V_PRACTICE | FAX_START_TIME | | | | |
| V_PRACTICE | FAX_STOP_TIME | | | | |
| V_PROVIDER_CHANGE_HISTORY | DATE_TIME | | | | |
| V_PROVIDER_CHANGE_HISTORY | PATIENT_ID | | | | |
| V_PROVIDER_CHANGE_HISTORY | NEW_PRIMARY_PROVIDER_ID | | | | |
| V_PROVIDER_CHANGE_HISTORY | NEW_CONTACT_PREFIX | | | | |
| V_PROVIDER_CHANGE_HISTORY | NEW_CONTACT_NAME | | | | |
| V_PROVIDER_CHANGE_HISTORY | PREVIOUS_CONTACT_PREFIX | | | | |
| V_PROVIDER_CHANGE_HISTORY | PREVIOUS_PRIMARY_PROVIDER_ID | | | | |
| V_PROVIDER_CHANGE_HISTORY | PREVIOUS_CONTACT_NAME | | | | |
| V_PROVIDER_CHANGE_HISTORY | LATEST_STATUS | | | | |
| V_PROVIDER_CHANGE_HISTORY | REASON_FOR_CHANGE | | | | |
| V_REPORT_LOG | REPORT_ID | | | | |
| V_REPORT_LOG | PATIENT_ID | | | | |
| V_REPORT_LOG | CONTACT_ID | | | | |
| V_REPORT_LOG | CLIENT_ID | | | | |
| V_REPORT_LOG | SEND_TIME | | | | |
| V_REPORT_LOG | OUTPUT_TYPE | | | | |
| V_REPORT_LOG | REPORT_STATUS | | | | |
| V_REPORT_LOG | REPORT_FILE | | | | |
| V_REPORT_LOG | REPORT_FILE_NAME | | | | |
| V_REPORT_LOG | CANNED_TEXT_COMBINATION | | | | |
| V_REPORT_LOG | FAX_JOB_ID | | | | |
| V_TEST_RESULT | UNIT | | | | |
| V_TEST_RESULT | INTERP_CODE | | | | |
| V_TEST_RESULT | NOTE | | | | |
| V_TEST_RESULT | STATUS | | | | |
| V_TEST_RESULT | TEST_ID | | | | |
| V_TEST_RESULT | FLOWSHEET_SENT | | | | |
| V_TEST_RESULT | PATIENT_ALERT_SENT | | | | |
| V_TEST_RESULT | RESULT_RANGE | | | | |
| V_TEST_RESULT | SERVICE_ORDER_ID | | | | |
| V_TEST_RESULT | RESULT_ID | | | | |
| V_TEST_RESULT | SERVICE_PROVIDER | | | | |
| V_TEST_RESULT | SERVICE_CODE | | | | |
| V_TEST_RESULT | PARENT_CODE | | | | |
| V_TEST_RESULT | ORDERING_PROV_ID | | | | |
| V_TEST_RESULT | PATIENT_ID | | | | |
| V_TEST_RESULT | RESULT_DATE | | | | |
| V_TEST_RESULT | REFERENCE_RANGE | | | | |
| VDIS_MONITOR_EMAIL_RCPT | ALERT_TYPE | | | | |
| VDIS_MONITOR_EMAIL_RCPT | EMAIL_ADDRESS | | | | |
| VDIS_MONITOR_EMAIL_RCPT | DESCRIPTION | | | | |
| VERMONT_SAMPLE | PATIENT_ID | | | | |
| VERMONT_SAMPLE | DT | | | | |
| VERMONT_SAMPLE | PI | | | | |
| VERMONT_SAMPLE_PERCENTAGE | LOW_PATIENT_PERCENTAGE | | | | |
| VERMONT_SAMPLE_PERCENTAGE | MEDIUM_PATIENT_PERCENTAGE | | | | |
| VERMONT_SAMPLE_PERCENTAGE | HIGH_PATIENT_PERCENTAGE | | | | |
| VERMONT_SAMPLE_PERCENTAGE | TEST_CODE | | | | |
| VM | VM1 | | | | |
| VM | VM2 | | | | |
| VM | VM3 | | | | |
| VM | VM4 | | | | |
| VM_HL7_MESSAGE_STRUCTURE | STRUCTURE_ID | | | | |
| VM_HL7_MESSAGE_STRUCTURE | MESSAGE_NAME | | | | |
| VM_HL7_MESSAGE_STRUCTURE | SEGMENT_ID | | | | |
| VM_HL7_MESSAGE_STRUCTURE | FIELD_NAME | | | | |
| VM_HL7_MESSAGE_STRUCTURE | SEGMENT_ORDER | | | | |
| VM_HL7_MESSAGE_STRUCTURE | FIELD_ORDER | | | | |
| VM_HL7_MESSAGE_STRUCTURE | COMPONENT_ORDER | | | | |
| VM_HL7_MESSAGE_STRUCTURE | SEGMENT_COUNT | | | | |
| VM_HL7_MESSAGE_STRUCTURE | REPEAT_COUNT | | | | |
| VM_HL7_MESSAGE_STRUCTURE | COMPONENT_COUNT | | | | |
| VM_HL7_MESSAGE_STRUCTURE | DEFAULT_VALUE | | | | |
| VM_HL7_MESSAGE_STRUCTURE | SEGMENT_REQUIRED | | | | |
| VM_HL7_MESSAGE_STRUCTURE | FIELD_REQUIRED | | | | |
| VM_HL7_MESSAGE_STRUCTURE | FIELD_FORMAT | | | | |
| VM_HL7_MESSAGE_STRUCTURE | FIELD_MAPPING | | | | |
| VSAMPLE | PATIENT_ID | | | | |
| VSAMPLE | VERMONT_SAMPLE | | | | |
| WEB_PAGE | WEB_PAGE_URL | | | | |
| WEB_PAGE | DESCRIPTION | | | | |
| WEB_PAGE | WEB_PAGE_ACCESS | | | | |
| WEB_PAGE | APPLICATION_FLAG | | | | |
| WEEKLY_LAB_PROJECT_TOTALS | WEEK_ENDING | | | | |
| WEEKLY_LAB_PROJECT_TOTALS | TOTAL_LOADED | | | | |
| WEEKLY_LAB_TEST_TOTALS | LAB_ID | | | | |
| WEEKLY_LAB_TEST_TOTALS | TEST_CODE | | | | |
| WEEKLY_LAB_TEST_TOTALS | WEEK_ENDING | | | | |
| WEEKLY_LAB_TEST_TOTALS | STATUS | | | | |
| WEEKLY_LAB_TEST_TOTALS | TOTAL_LOADED_BY_RESULT_DATE | | | | |
| WEEKLY_LAB_TEST_TOTALS | TOTAL_LOADED_BY_PARSE_DATE | | | | |
| WEEKLY_LAB_TEST_TOTALS | TOTAL_LOADED_BY_COLLECT_DATE | | | | |
| WORKS_AT_HOUSES | LOCATION_ID | | | | |
| WORKS_AT_HOUSES | CONTACT_ID | | | | |

The invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A method for clinical decision support comprising:
retrieving patient clinical information from a remote data site;
performing clinical information interpretation by a guideline-based algorithm; and
reporting the clinical information interpretation to a healthcare provider and/or a patient.

2. The method of claim 1, wherein the patient clinical information is retrieved over a secure network.

3. The method of claim 1, wherein the clinical decision support comprises automated patient medical report generation.

4. The method of claim 1, wherein the method is used for managing a chronic medical condition of a patient.

5. The method of claim 4, wherein the chronic medical condition is selected from the list consisting of: diabetes mellitus, cholesterol related disorder, hepatitis, thyroid related disorder and cancer.

6. The method of claim 4, wherein the chronic medical condition is diabetes mellitus.

7. The method of claim 1, wherein the patient clinical information is selected from the group consisting of: laboratory test data, X-ray data, examination and diagnosis.

8. The method of claim 7, wherein the patient clinical information is laboratory test data.

9. The method of claim 8, wherein the laboratory test data is results from tests selected from the list consisting of: AlC, serum lipid, urinary microalbumin to creatinine ratio (MCR), and serum creatinine.

10. The method of claim 1, wherein the remote data site is a laboratory.

11. The method of claim 1, wherein the remote data site is a point-of-care testing facility.

12. The method of claim 1, wherein the step of retrieving the patient clinical information is carried out at a regular time interval.

13. The method of claim 12, wherein the regular time interval is at least once a day.

14. The method of claim 1, wherein the guideline-based algorithm is developed from a chronic care model.

15. The method of claim 1, wherein the reporting of clinical information interpretation is carried out by telephone, pager, e-mail, facsimile, mail or via an electronic health record interface.

16. The method of claim 1, wherein the reporting of clinical information interpretation comprises a facsimile report to the healthcare provider.

17. The method of claim 1, wherein the reporting of clinical information interpretation comprises a mail report for the patient.

18. The method of claim 1, wherein the reporting of the interpretation of clinical information comprises a facsimile report to the health-care provider and a mail report to the patient.

19. An automated electronic system for clinical decision support comprising:
a storage device for storing patient clinical information;
a processor for automatically retrieving the patient clinical information from medical facilities and
interpreting the patient clinical information by a guideline-based algorithm; and
a processor for sending the clinical information interpretation to a healthcare provider and/or patient.

20. The system of claim 19, wherein the clinical decision support is a patient medical report.

21. The system of claim 19, wherein the patient clinical information is patient laboratory test data.

22. A computer program product for clinical decision support comprising
computer readable code for generating and maintaining a patient registry database;
computer readable code for retrieving clinical information from a remote data site;
computer readable code for interpreting the clinical information and
computer readable code for reporting the interpretation of the clinical information.

23. A computer program product of claim 22, wherein the computer program product for clinical decision support is a program for automated medical reporting.

24. The computer readable code of claim 22, wherein the retrieving of patient clinical information is carried out at regular time intervals.

25. The computer readable code of claim 22, wherein the patient clinical information is laboratory test data.

26. The computer readable code of claim 22, wherein the interpreting of patient clinical information is guideline-based.
